(19) United States
(12) Patent Application Publication
(10) Pub. No.: US 2025/0335785 A1
(43) Pub. Date: Oct. 30, 2025

(54) SYSTEMS AND METHODS FOR MACHINE LEARNING-BASED GENOME ANNOTATION

(71) Applicants: InstaDeep Ltd, London (GB); BioNTech SE, Mainz (DE)

(72) Inventors: Thomas Pierrot, Boston, MA (US); Bernardo P. De Almeida, Paris (FR); Guillaume Richard, Paris (FR); Hugo Dalla-Torre, Paris (FR); Alexandre Laterre, London (GB); Karim Beguir, London (GB); Lorenz Johann Leopold Hexemer, Nierstein (DE); Stefan Jean Yvon Laurent, Cologne (DE); Maren Lang, Mainz (DE); Priyanka Pandey, Mainz (DE); Ugur Sahin, Mainz (DE)

(21) Appl. No.: 19/075,335

(22) Filed: Mar. 10, 2025

Related U.S. Application Data
(60) Provisional application No. 63/563,903, filed on Mar. 11, 2024.
(60) Provisional application No. 63/683,682, filed on Aug. 15, 2024.
(60) Provisional application No. 63/701,114, filed on Sep. 30, 2024.

(51) Int. Cl. G06N 3/123; G16B 40/30

(52) U.S. Cl. CPC G06N 3/123; G16B 40/30

(57) ABSTRACT

The present disclosure, among other things, provides machine-learning technologies for identifying and localizing particular genomic elements (e.g., gene elements and/or regulatory elements) within nucleotide sequences, such as DNA and/or RNA sequences. In certain embodiments, similar to the manner in which image processing methods can be used to localize particular objects in images at pixel level resolution, referred to as “segmentation,” systems and methods of the present disclosure predict presence and locations of certain genomic elements within nucleotide sequences, thereby “segmenting” nucleotide sequences. Accordingly, genomic element segmentation technologies described herein may be used to generate annotations that identify and label portions of nucleotide sequences according to their predicted (e.g., via machine learning models described herein) function—e.g., as protein-coding genes, untranslated regions, splice sites, promotors, enhancers, etc. Among other things, these genomic annotations may be used to inform underlying biological processes driving diseases and facilitate development of new therapies.

### CROSS-REFERENCE TO RELATED APPLICATIONS

This application claims priority to U.S. Provisional Patent Application No. 63/563,903, filed Mar. 11, 2024, the title of which is “Segmentation of nucleotide sequences,” to U.S. Provisional Patent Application No. 63/683,682, filed Aug. 15, 2024, the title of which is “Systems and methods for language model-based genome annotation”, and to U.S. Provisional Patent Application No. 63/701,114, filed Sep. 30, 2024, the title of which is “Systems and methods for language model-based genome annotation”, the content of each of which is incorporated herein by reference in its entirety.

### BACKGROUND

The ability to determine the roles and underlying functions of, and interplay between, genetic information encoded by the billions of nucleotides that make up the human genetic code is central to a foundational understanding of disease and lays at the corner stone of developing various therapies. Yet, despite recent advances in genomics, important steps in genomic analysis, such as identifying and characterizing various protein coding and regulatory elements within DNA sequences, continue to present significant challenges.

### SUMMARY

The present disclosure, among other things, provides machine-learning technologies for identifying and localizing particular genomic elements (e.g., gene elements and/or regulatory elements) within nucleotide sequences, such as DNA sequences. In certain embodiments, similar to the manner in which image processing methods can be used to localize particular objects in images at pixel level resolution, referred to as “segmentation,” systems and methods of the present disclosure predict presence and locations of certain genomic elements within nucleotide sequences, thereby “segmenting” nucleotide sequences. Accordingly, genomic element segmentation technologies described herein may be used to generate annotations that identify and label portions of nucleotide sequences according to their predicted (e.g., via machine learning models described herein) function—e.g., as protein-coding genes, untranslated regions, splice sites, promotors, enhancers, etc. Among other things, these genomic annotations may be used to inform underlying biological processes driving diseases and facilitate development of new therapies.

In certain embodiments, genomic element segmentation technologies of the present disclosure utilize machine learning models to generate predictions for a given nucleotide sequence that identify which particular genomic elements it comprises and where these genomic elements are located (within the given sequence). For example, systems and methods described herein may annotate nucleotide sequences by assigning labels to various sets of (e.g., consecutive) nucleotides (e.g., subsequences) that are identified as belonging to particular genomic elements. Subsequences of nucleotides and their assigned labels may be determined using a machine learning model that receives nucleotide sequence data as input and generates, as output, likelihood values representing, for each group of one or more nucleotides, a predicted likelihood of belonging to a particular genomic element.

In this manner, a machine learning model may generate quantitative predictions—e.g., numerical likelihoods—about whether particular nucleotides or groups thereof act as particular genomic elements. These predictions may be generated for one or multiple genomic elements, including various gene and/or regulatory elements, allowing elements such as (without limitation) protein-coding genes, long non-coding RNAs (lncRNAs), 5′ untranslated regions (5′ UTRs), 3′ untranslated regions (3′ UTRs), exons, introns, splice sites (e.g., splice donor sites and/or splice acceptor sites), polyadenylation (polyA) signal regions, promoters (e.g., tissue-invariant promotors and/or tissue-specific promotors), enhancers (e.g., tissue-invariant enhancers and/or tissue-specific enhancers) CCCTC-binding factor (CTCF)-binding sites, and the like, to be identified. For example, machine learning models of the present disclosure may comprise or generate a plurality of output channels, each corresponding to a particular genomic element and comprising, for each nucleotide and/or group of one or more nucleotides, a predicted likelihood that it (the nucleotide and/or group of one or more nucleotides) belongs to the particular genomic element. Multiple channels of genomic element predictions may thus be generated and likelihoods within each channel may be evaluated to assign genomic labels to individual nucleotides and/or sets of nucleotides.

As described in further detail herein, likelihoods may be generated for each individual nucleotide in a sequence and/or on a token-by-token basis, with each token representing a set of k consecutive nucleotides, where k is an integer (e.g., greater than or equal to one). In this manner, beyond simply detecting presence of various genomic element(s) within a given sequence, genomic element segmentation technologies of the present disclosure localize them at high resolution, down to the single nucleotide level.

Among other things, in certain embodiments, machine learning models of the present disclosure incorporate language models (LMs) that operate on nucleotide sequence data, treating the combination of nucleotides in a given nucleotide sequence, similar to how natural language (e.g., English language) models treat combinations of words in sentences. As described herein, genomic LMs may be trained on nucleotide sequence data in an unsupervised fashion, via techniques such as masked token prediction or next token prediction, allowing them to leverage the wealth of raw (i.e., not necessarily labeled) sequence data made available through modern next generation sequencing (NGS) technologies and various research initiatives. As a consequence of these training procedures, genomic LMs ‘learn’ to generate (e.g., internally) higher-level representations (e.g., high-dimensional numerical vectors)—referred to as embeddings—of nucleotides and/or nucleotide sequences. As shown, for example in H. Dalla-Torre et al., “The Nucleotide Transformer: Building and Evaluating Robust Foundation Models for Human Genomics,” bioRxiv, 2023, these embeddings encode context and detailed information about nucleotide sequences.

In certain embodiments, genomic LMs may be used in conjunction with a second model (e.g., a second sub-model), such as a segmentation head. The genomic LM may be or function as an encoder, receiving nucleotide sequence data as input and generating embeddings that may, in turn, be used as input to a segmentation head that generates, as output, likelihood values for various genomic elements as described herein. In this way, a genomic LM encoder can be trained to create embeddings that include and/or encode key features of genomic sequence elements at the outset, using unlabeled data and in an unsupervised fashion. A segmentation head may then be trained using labeled data, to localize various genomic elements. Although the segmentation head utilizes a supervised training approach, it takes advantage of the information-rich embeddings generated by an LM encoder. In this way, the segmentation head is provided with a ‘head-start’, contrasting with other approaches where segmentation models operate directly on a nucleotide sequence (e.g., using simples rule to encode individual nucleotides). Among other things, this approach allows downstream models, such as segmentation techniques, that traditionally require labeled data, to take advantage of the abundance of unlabeled sequence data, thereby allowing for highly accurate models to be obtained even with limited quantities of labeled data.

Additionally or alternatively, in certain embodiments, machine learning technologies of the present disclosure may utilize certain insights and approaches described herein to provide (e.g., further) improvements in performance. For example, certain embodiments described herein employ multi-task models in which a single segmentation head is used to annotate nucleotide sequences with multiple genomic elements at the same time. As described herein, not only does this multi-task approach streamline model architecture, but, moreover, it leverages transfer learning whereby benefits of shared knowledge across multiple tasks can lead to improved performance. In certain embodiments, approaches described extend lengths of nucleotide sequences that can be handled (e.g., in one shot) by machine learning models. As described herein, in certain embodiments, an ability to annotate nucleotide sequence with increased length (e.g., up to 100 kb at once) can improve performance by allowing machine learning models to benefit from additional context. annotating nucleotide sequences belonging to various species may benefit from shared knowledge across species that, in turn, may lead to improved performance.

Genomic element likelihood values and/or annotated sequence data provided via genomic segmentation techniques described herein may be displayed, stored, or provided for further downstream processing/analysis, serving as a distinct and new result that can be leveraged for, for example, diagnostics and treatment development. Among other things, as described herein, annotated sequence data and/or genomic element likelihood values generated via the techniques of the present disclosure can be used to evaluate impact of sequence variants on genomic elements, providing a tool to study effects of mutation on genomic elements for various diseases, such as cancer.

Accordingly, by providing technologies for accurately annotating and evaluating genomic elements in a biological sequence in-silico, methods and systems described herein can dramatically reduce the burden of extensive trial and error experimentation, allowing for improvements in efficacy with reduced costs and time to development.

In some aspects, the present disclosure provides methods for determining locations of one or more genomic elements within a nucleotide sequence (e.g., a DNA sequence, an RNA sequence). In certain embodiments, provided methods comprise: (a) receiving, by a processor of a computing device, nucleotide sequence data representing a sequence of a plurality of nucleotides; (b) determining, by the processor, using a machine learning model and based on the nucleotide sequence data, a plurality of likelihood values, wherein each likelihood value is associated with (i) a particular nucleotide of the sequence and (ii) a particular one of the one or more genomic element(s), and wherein each likelihood value represents and/or quantifies a likelihood that the particular nucleotide is part of [e.g., is part of a subsequence (plurality of nucleotides) that functions as (for example by coding for a particular protein, coding for a particular RNA, binding one or more transcription factors, etc.)] the particular genomic element with which the likelihood value is associated; (c) determining and/or assigning, by the processor, one or more genomic element labels to each of at least a portion of the plurality of nucleotides, based at least in part on the plurality of likelihood values (e.g., using a function, using a threshold value, using a classifier, etc.), thereby creating an annotated sequence data comprising the nucleotide sequence data together with the assigned genomic element labels; and (d) storing, by the processor, the annotated sequence data and/or providing, by the processor, the annotated sequence data for display, and/or further processing.

In certain embodiments, a nucleotide sequence data represents a deoxyribonucleic acid (DNA) sequence and/or a ribonucleic acid (RNA) sequence.

In certain embodiments, a machine learning model receives as input and/or generates (e.g., internally) a tokenized representation of the sequence of the plurality of nucleotides [e.g., wherein the nucleotide sequence data comprises a sequence of tokens, each token of the sequence of tokens corresponding to (i) a (e.g., non-overlapping) set of consecutive nucleotides (e.g., a k-mer, where k is an integer, e.g., 1, 2, 3, 4, 5, 6, 8, 10, etc.) of the sequence or (ii) a particular one of a finite number of standard non-sequence tokens (e.g., class [CLS], pad [PAD], mask [MASK])].

In certain embodiments, nucleotide sequence data has a length of at least 100 kilobases (kb) (e.g., at least 50 kb, at least 30 kb, at least 20 kb, at least 10 kb, at least 6 kb, at least 3 kb).

In certain embodiments, provided methods comprise sub-dividing the nucleotide sequence data into two or more partitions, each of the two or more partitions corresponding to a (e.g., distinct, non-overlapping) sub-sequence of the plurality of nucleotides. In certain embodiments, provided methods comprise, at step (b), using the machine learning model to determine a corresponding subset of the likelihood values for each partition (e.g., separately) (e.g., wherein each partition is provided as input to the machine learning model, and a corresponding subset of the likelihood values generated as output, separately/independently).

In certain embodiments, one or more genomic elements comprise five (5) or more genomic elements (e.g., 10 or more genomic elements; e.g., 14 or more genomic elements).

In certain embodiments, one or more genomic elements comprise one or more gene elements (e.g., protein-coding genes, lncRNAs, 5′UTR, 3′UTR, exon, intron, splice acceptor, donor sites).

In certain embodiments, one or more genomic elements comprise one or more regulatory elements (e.g., polyA signal, tissue-invariant and tissue-specific promoters and/or enhancers, CTCF-bound sites).

In certain embodiments, one or more of the genomic elements are associated with (e.g., a presence of) a disease (e.g., cancer).

In certain embodiments, a machine learning model comprises (i) an encoder and (ii) a segmentation head.

In certain embodiments, an encoder is a pre-trained (e.g., foundation) model, having been previously trained, separately from the segmentation head (e.g., in combination with one or more output layers).

In certain embodiments, an encoder comprises one or more transformer layers (e.g., wherein the encoder is or comprises a language model).

In certain embodiments, an encoder comprises one or more convolutional layers.

In certain embodiments, an encoder comprises (i) one or more convolutional layers and (ii) one or more transformer layers [e.g., wherein at least a portion of the one or more convolutional layers precede the one or more transformer layers (e.g., wherein the portion of the one or more convolutional layers are arranged as a first (e.g., down-sampling) convolutional block that down-samples the input to the encoder to generate an intermediate (e.g., down-sampled) representation, followed by the one or more transformer layers); e.g., wherein at least a portion of the one or more convolution layers follow the one or more transformer layers and receive a first resolution embedding as input and generate, as output, a second, higher resolution embedding].

In certain embodiments, step (b) comprises generating, via the encoder, one or more embeddings (e.g., a set of embedding vectors) based on the nucleotide sequence data and/or a tokenized version thereof. In certain embodiments, step (b) comprises determining, via the segmentation head, the plurality of likelihood values, based on the one or more embeddings.

In certain embodiments, step (b) comprises providing at least a portion of the nucleotide sequence data and/or a tokenized version thereof as input to the encoder to generate, via the encoder model, the one or more embeddings based on received input. In certain embodiments, step (b) comprises using the one or more embeddings as input to the segmentation head to generate, via the segmentation head, the plurality of likelihood values.

In certain embodiments, an encoder is or comprises a pre-trained neural network (e.g., a transformer-based neural network) having been trained, at least in part (e.g., in combination with a supervised training approach; e.g., entirely) in an un-supervised fashion using a training dataset comprising a plurality of example nucleotide sequences [e.g., the pre-trained neural network having been trained to predict most likely tokens/nucleotides at masked positions in the plurality of example nucleotide sequences (e.g., masked language modeling (MLM))].

In certain embodiments, an encoder is or comprises a pre-trained neural network (e.g., a transformer-based neural network) having been trained, at least in part (e.g., in combination with an un-supervised training approach; e.g., entirely), in a supervised fashion using a training dataset comprising a plurality of example nucleotide sequences and, for each example nucleotide sequence, a corresponding set of target output values [e.g., the pre-trained neural network having been trained to (e.g., repeatedly) receive, as input, an example nucleotide sequence and generate, as output, a predicted output value matching the target output value (e.g., and evaluated and/or refined based on a comparison between the predicted output value and the target output value)].

In certain embodiments, a segmentation head is or comprises a convolutional neural network (CNN) [e.g., a U-net architecture (e.g., a one-dimensional U-net architecture)].

In certain embodiments, a machine learning model comprises (i) a language model-based encoder and (ii) a segmentation head.

In certain embodiments, step (b) comprises generating, via a language model-based encoder, one or more embeddings (e.g., a set of embedding vectors) based on the nucleotide sequence data and/or a tokenized version thereof. In certain embodiments, step (b) comprises determining, via the segmentation head, the plurality of likelihood values, based on the one or more embeddings.

In certain embodiments, step (b) comprises providing at least a portion of the nucleotide sequence data and/or a tokenized version thereof as input to the language model-based encoder to generate, via the language model-based encoder, the one or more embeddings based on received input. In certain embodiments, step (b) comprises using the one or more embeddings as input to the segmentation head to generate, via the segmentation head, the plurality of likelihood values.

In certain embodiments, a segmentation head is or comprises a convolutional neural network (CNN) [e.g., a U-net architecture (e.g., a one-dimensional U-net architecture)].

In certain embodiments, a language model-based encoder is or comprises a pre-trained neural network (e.g., a transformer-based neural network) having been trained in an un-supervised fashion using a training dataset comprising a plurality of example nucleotide sequences [e.g., the pre-trained neural network having been trained to predict most likely tokens/nucleotides at masked positions in the plurality of example nucleotide sequences (e.g., masked language modeling (MLM))].

In certain embodiments, a machine learning model has been trained using a training dataset comprising example human nucleotide sequences.

In certain embodiments, a machine learning model has been trained using a training dataset comprising example nucleotide sequences from a plurality of different species (e.g., two species, five species) [e.g., mouse (mm10), chicken (galGal6), fly (dm6), zebrafish (danRer11) and worm (ce11)].

In certain embodiments, nucleotide sequence data represents a nucleotide sequence for a particular species that is not one of the plurality of different species from which the example nucleotides sequences used to train the machine learning model were obtained (e.g., the machine learning model performs zero-shot species inference) [e.g., gorilla (gorGor4), macaque (Mnem 1), rat (mRatBN7), beaver (can genome v1), chinchilla (ChiLan1), whale (ASM228892v3), cat (Felis catus 9), canary (SCA1), tetradon (T ET RAODON8), anemonefish (AmpOce1), trout (f SalT ru1) and Ciona intestinalis (KH)].

In certain embodiments, a length of nucleotide sequence data (e.g., at least 100 kb, at least 30 kb, at least 20 kb, at least 10 kb, at least 6 kb, at least 3 kb) is greater than a length of example nucleotide sequences (e.g., at least 100 kb, at least 30 kb, at least 20 kb, at least 10 kb, at least 6 kb, at least 3 kb) used for training the machine learning model (e.g., the machine learning model performs zero-shot context extension).

In certain embodiments, step (d) comprises determining, for each nucleotide, a genomic element associated with a maximum likelihood value.

In certain embodiments, step (d) comprises comparing the plurality of likelihood values to one or more threshold values.

In certain embodiments, step (c) comprises identifying, by the processor, one or more subsequence(s) within the nucleotide sequence data and determining, by the processor, an assigned genomic element label for each of the one or more subsequences based at least in part on the plurality of likelihood values.

In certain embodiments, step (d) comprises using the annotated sequence data to develop a therapy (e.g., a therapeutic, a genetic variant) (e.g., targeting an identified genomic element within the genomic sequence).

In certain embodiments, step (d) comprises using the annotated sequence data for detection, and/or prognosis of a diseases (e.g., cancer).

In some aspects, the present disclosure provides methods for determining locations of genomic elements within a nucleotide sequence. In certain embodiments, provided methods comprise: (a) receiving, by a processor of a computing device, nucleotide sequence data representing a nucleotide sequence comprising a plurality of nucleotides; (b) determining, by the processor, using a machine learning model and based on the nucleotide sequence data, a plurality of likelihood values, wherein each likelihood value is associated with (i) a particular group of one or more nucleotides of the nucleotide sequence and (ii) a particular one of a plurality of genomic elements, and wherein, each likelihood value represents and/or quantifies a likelihood that at least a portion of the one or more nucleotides of the particular group is/are part of the particular one of the plurality of genomic elements with which it is associated; (c) determining and/or assigning, by the processor, one or more genomic element labels to each of at least a portion of the plurality of nucleotides, based at least in part on the plurality of likelihood values (e.g., using a function, using a threshold value, using a classifier), thereby creating an annotated sequence data comprising the nucleotide sequence data together with the assigned genomic element labels; and (d) storing, by the processor, the annotated sequence data and/or providing, by the processor, the annotated sequence data for display, and/or further processing.

In some aspects, the present disclosure provides methods for determining locations of genomic elements within a genomic sequence. In certain embodiments, provided methods comprise: (a) receiving, by a processor of a computing device, nucleotide sequence data representing a sequence comprising a plurality of nucleotides; (b) determining, by the processor, using a machine learning model and based on the nucleotide sequence data, a plurality of likelihood values that (e.g., collectively) measure a probability of each nucleotide of the sequence belonging to one or more of particular genomic elements, wherein the machine learning model comprises (i) an encoder model (e.g., comprising one or more transformer layers; e.g., a language model-based encoder) and (ii) a segmentation head; (c) creating, by the processor, annotated sequence data comprising identifications of one or more genomic elements based on the likelihood values; and (d) storing, by the processor, the annotated sequence data and/or providing, by the processor, the annotated sequence data for display, and/or further processing.

In certain embodiments, a segmentation head comprises a convolutional neural network (CNN) [e.g., a U-net architecture (e.g., a one-dimensional U-net architecture)].

In certain embodiments, an encoder is a pre-trained (e.g., foundation) model, having been previously trained, separately from the segmentation head (e.g., in combination with one or more output layers).

In certain embodiments, an encoder comprises one or more transformer layers (e.g., wherein the encoder is or comprises a language model).

In certain embodiments, an encoder is a language model-based encoder.

In certain embodiments, an encoder comprises one or more convolutional layers.

In certain embodiments, an encoder comprises (i) one or more convolutional layers and (ii) one or more transformer layers [e.g., wherein at least a portion of the one or more convolutional layers precede the one or more transformer layers (e.g., wherein the portion of the one or more convolutional layers are arranged as a first (e.g., down-sampling) convolutional block that down-samples the input to the encoder to generate an intermediate (e.g., down-sampled) representation, followed by the one or more transformer layers); e.g., wherein at least a portion of the one or more convolution layers follow the one or more transformer layers and receive a first resolution embedding as input and generate, as output, a second, higher resolution embedding].

In certain embodiments, step (b) comprises generating, via the encoder (e.g., a language model-based encoder; e.g., an encoder comprising one or more convolutional layers), one or more embeddings (e.g., a set of embedding vectors) based on the nucleotide sequence data and/or a tokenized version thereof. In certain embodiments, step (b) comprises determining, via the segmentation head, the plurality of likelihood values, based on the one or more embeddings.

In certain embodiments, step (b) comprises providing at least a portion of the nucleotide sequence data and/or a tokenized version thereof as input to the encoder to generate, via the encoder, the one or more embeddings based on received input. In certain embodiments, step (b) comprises using the one or more embeddings as input to the segmentation head to generate, via the segmentation head, the plurality of likelihood values.

In certain embodiments, an encoder is or comprises a pre-trained neural network (e.g., a transformer-based neural network) having been trained in an un-supervised fashion using a training dataset comprising a plurality of example nucleotide sequences [e.g., wherein the encoder is a language model-based and the pre-trained neural network having been trained to predict most likely tokens/nucleotides at masked positions in the plurality of example nucleotide sequences (e.g., masked language modeling (MLM))].

In certain embodiments, an encoder is or comprises a pre-trained neural network (e.g., a transformer-based neural network) having been trained, at least in part (e.g., in combination with an un-supervised training approach; e.g., entirely), in a supervised fashion using a training dataset comprising a plurality of example nucleotide sequences and, for each example nucleotide sequence, a corresponding set of target output values [e.g., the pre-trained neural network having been trained to (e.g., repeatedly) receive, as input, an example nucleotide sequence and generate, as output, a predicted output value matching the target output value (e.g., and evaluated and/or refined based on a comparison between the predicted output value and the target output value)].

In certain embodiments, step (b) comprises providing at least a portion of the nucleotide sequence data and/or a tokenized version thereof as input to the encoder to generate, via the encoder, the one or more embeddings based on the received input [e.g., wherein the encoder is a language model-based encoder that (i) receives, as input, a sequence of tokens, wherein each set of one or more consecutive nucleotides is represented by a particular token, and (ii) generates (e.g., as output) the embeddings. In certain embodiments, one or more embeddings comprise(s) a set of embedding vectors, each embedding vector corresponding to a particular token of the sequence of tokens received as input by the language model-based encoder].

In certain embodiments, a method comprises using the one or more embeddings as input to the segmentation head to generate, via the segmentation head, the plurality of likelihood values (e.g., wherein the segmentation head receives, as input, the one or more embedding representations and generates the plurality of likelihood values as output).

In certain embodiments, a language model-based encoder comprises a context length extension method (e.g., and rotary positional embeddings) to rescale a frequency used in rotary positional embeddings.

In some aspects, the present disclosure provides systems for determining locations of one or more genomic elements within a nucleotide sequence (e.g., a DNA sequence, an RNA sequence). In certain embodiments, a system comprises: a processor of a computing device; and memory having instructions stored thereon, wherein the instructions, when executed by the processor, cause the processor to: (a) receive nucleotide sequence data representing a sequence of a plurality of nucleotides; (b) determine, using a machine learning model and based on the nucleotide sequence data, a plurality of likelihood values, wherein each likelihood value is associated with (i) a particular nucleotide of the sequence and (ii) a particular one of the one or more genomic element(s), and wherein each likelihood value represents and/or quantifies a likelihood that the particular nucleotide is part of the particular genomic element with which the likelihood value is associated; (c) determine and/or assign one or more genomic element labels to each of at least a portion of the plurality of nucleotides, based at least in part on the plurality of likelihood values (e.g., using a function, using a threshold value, using a classifier, etc.), thereby creating an annotated sequence data comprising the nucleotide sequence data together with the assigned genomic element labels; and (d) store the annotated sequence data and/or provide the annotated sequence data for display and/or further processing.

In some aspects, the present disclosure provides systems for determining locations of genomic elements within a nucleotide sequence, the system comprising: a processor of a computing device; and memory having instructions stored thereon, wherein the instructions, when executed by the processor, cause the processor to: (a) receive nucleotide sequence data representing a nucleotide sequence comprising a plurality of nucleotides; (b) determine, using a machine learning model and based on the nucleotide sequence data, a plurality of likelihood values, wherein each likelihood value is associated with (i) a particular group of one or more nucleotides of the nucleotide sequence and (ii) a particular one of a plurality of genomic elements, and wherein each likelihood value represents and/or quantifies a likelihood that at least a portion of the one or more nucleotides of the particular group is/are part of the particular one of the plurality of genomic elements with which it is associated; (c) determine and/or assign one or more genomic element labels to each of at least a portion of the plurality of nucleotides, based at least in part on the plurality of likelihood values (e.g., using a function, using a threshold value, using a classifier, etc.), thereby creating an annotated sequence data comprising the nucleotide sequence data together with the assigned genomic element labels; and (d) store the annotated sequence data and/or provide the annotated sequence data for display and/or further processing.

In some aspects, the present disclosure provides systems for determining locations of genomic elements within a genomic sequence, the system comprising: a processor of a computing device; and memory having instructions stored thereon, wherein the instructions, when executed by the processor, cause the processor to: (a) receive nucleotide sequence data representing a sequence comprising a plurality of nucleotides; (b) determine, using a machine learning model and based on the nucleotide sequence data, a plurality of likelihood values that (e.g., collectively) measure a probability of each nucleotide of the sequence belonging to one or more of particular genomic elements, wherein the machine learning model comprises (i) an encoder and (ii) a segmentation head; (c) create annotated sequence data comprising identifications of one or more genomic elements based on the likelihood values; and (d) store the annotated sequence data and/or provide the annotated sequence data for display and/or further processing.

In some aspects, the present disclosure provides computer-implemented methods comprising: obtaining sequence data representing a nucleotide sequence (e.g., a sequence of tokens, each token corresponding to one or more nucleotides in the nucleotide sequence); processing the sequence data using an encoder portion of a machine-learning model to generate a sequence of embeddings, each embedding corresponding to one or more nucleotides in the nucleotide sequence; processing the sequence of embeddings using a segmentation head of the machine learning model to determine, for each nucleotide in the nucleotide sequence, a respective set of values each indicting whether that nucleotide is predicted to be associated with a respective class of genomic element.

In certain embodiments, an encoder portion of the machine-learning model is a transformer model pre-trained to predict masked tokens in sequences of tokens corresponding to ground truth nucleotide sequences.

In certain embodiments, an encoder portion of the machine learning model comprises one or more convolutional layers.

In certain embodiments, sequence data represents the nucleotide sequence via a sequence of tokens, each token corresponding to one or more nucleotides in the nucleotide sequence and the encoder portion of the machine-learning model has been pre-trained on sequences corresponding to a first number of tokens to generate rotary position embeddings supporting sequences of up to the first number of tokens. In certain embodiments, a sequence of tokens includes a second number of tokens that is greater than the first number of tokens. In certain embodiments, a method comprises generating the sequence of embeddings comprises extending a context length of the encoder from the first number of tokens to the second number of tokens.

In certain embodiments, extending a context length of an encoder comprises rescaling a frequency used to generate rotary position embeddings from a first frequency corresponding to the first number of tokens to a second frequency corresponding to the second number of tokens.

In certain embodiments, a first number of tokens corresponds to a nucleotide sequence of no greater than 12 kilobases, and the second number of tokens corresponds to a nucleotide sequence of at least 20 kilobases, or at least 30 kilobases, or at least 50 kilobases.

In certain embodiments, provided methods comprise obtaining annotation data indicating whether each nucleotide in the nucleotide sequence is associated with each of a plurality of classes of genomic element. In certain embodiments, provided methods comprise updating parameters of the machine-learning model to reduce a loss function depending on a departure of the respective set of values for each nucleotide from corresponding indications in the annotation data.

In certain embodiments, a loss function is a focal loss function.

In certain embodiments, a nucleotide sequence is a first nucleotide sequence. In certain embodiments, a machine-learning has been trained on a set of annotated nucleotide sequences of a first species. In certain embodiments, a first nucleotide sequence is a nucleotide sequence of a second species different from the first species.

In certain embodiments, a first species is human.

In certain embodiments, respective classes include at least some of the following list of classes: protein-coding gene; lncRNA; 5′UTR; 3′UTR; exon; intron, splice acceptor; donor sites; polyA signal; tissue-invariant promoter; tissue-specific promoter; tissue-invariant enhancer; tissue-specific enhancer; and CTCF-bound site.

In certain embodiments, a given embedding in the sequence of embeddings corresponds to a 6-mer of nucleotides in the nucleotide sequence.

In certain embodiments, a respective set of values for each nucleotide is indicative of predicted probabilities of that nucleotide being associated with each of the respective classes of genomic element.

In certain embodiments, provided methods comprise determining that each nucleotide in the nucleotide sequence for which a given value of the respective set of values exceeds a detection threshold is associated with a corresponding class of genomic element.

In certain embodiments, a segmentation head has a 1D U-net architecture comprising at least one downsampling convolutional block and at least one upsampling convolutional block.

In certain embodiments, a 1D U-net architecture comprises skip connections between the at least one downsampling convolutional block and at least one upsampling convolutional block.

In certain embodiments, step (a) comprises obtaining a sequence of tokens by processing the sequence data using a tokenizer.

In some aspects, the present disclosure provides systems comprising: one or more processors; and one or more non-transitory computer-readable media storing: a machine-learning model comprising an encoder portion and a segmentation head; and instructions which, when executed by the one or more processors, cause the one or more processors to carry out a method presented herein.

In some aspects, the present disclosure provides a computer program product comprising instructions which, when the program is executed by a computer, cause the computer to carry out a method presented herein.

In certain embodiments, a computer program product comprises one or more non-transitory computer-readable media storing the instructions.

In some aspects, the present disclosure provides methods [e.g., for predicting isoforms (e.g., RNA isoforms, protein isoforms, peptide isoforms) due to alternative splicing events from nucleotide sequence data]. In certain embodiments, provided methods comprise: (a) receiving, by a processor of a computing device, nucleotide sequence data representing a sequence of a plurality of nucleotides; (b) determining, by the processor, using a machine learning model and based on the nucleotide sequence data, a plurality of likelihood values, wherein each likelihood value is associated with (i) a particular nucleotide of the sequence and (ii) a particular one of the one or more genomic element(s), and wherein each likelihood value represents and/or quantifies a likelihood that the particular nucleotide is part of [e.g., is part of a subsequence (plurality of nucleotides) that functions as, for example by coding for a particular protein, RNA, binding one or more transcription factors, etc.] the particular genomic element with which the likelihood value is associated; (c) determining, by the processor, for at least a portion of the nucleotide sequence data, two or more predicted isoforms (e.g., predicted to result from alternative splicing) using the plurality of likelihood values; and (d) storing, by the processor, data representing the two or more predicted isoforms and/or providing, by the processor, data representing the two or more predicted isoforms for display, and/or further processing.

In certain embodiments, provided methods comprise identifying, by the processor, one or more neoantigen and/or neoepitope candidates based at least in part on the data representing the two or more predicted isoforms [e.g., selecting, by the processor, at least a portion of one of the two or more predicted isoforms for inclusion a pharmaceutical composition].

In some aspects, the present disclosure provides methods (e.g., for predicting impact of one or mutations to a nucleotide sequence on alternative splicing events). In certain embodiments, provided methods comprise: (a) receiving, by a processor of a computing device, first nucleotide sequence data representing a first sequence of a plurality of nucleotides; (b) determining, by the processor, using a machine learning model and based on the first nucleotide sequence data, a first plurality of likelihood values, wherein each likelihood value of the first plurality of likelihood values is associated with (i) a particular nucleotide of the first sequence and (ii) a particular one of the one or more genomic element(s) and wherein each likelihood value represents and/or quantifies a likelihood that the particular nucleotide is part of [e.g., is part of a subsequence (plurality of nucleotides) that functions as, for example by coding for a particular protein, RNA, binding one or more transcription factors, etc.] the particular genomic element with which the likelihood value is associated; (c) receiving, by a processor of a computing device, second nucleotide sequence data representing a second nucleotide sequence corresponding to a mutated version of the first nucleotide sequence [e.g., an identification (e.g., a list) of mutations to the first nucleotide sequence; e.g., second sequence data representing the second, mutated, nucleotide sequence]; (d) determining, by the processor, using the machine learning model and based on the second nucleotide sequence data, a second plurality of likelihood values, wherein each likelihood value of the second plurality of likelihood values is associated with (i) a particular nucleotide of the second sequence and (ii) a particular one of the one or more genomic element(s) and wherein each likelihood value represents and/or quantifies a likelihood that the particular nucleotide is part of [e.g., is part of a subsequence (plurality of nucleotides) that functions as, for example by coding for a particular protein, RNA, binding one or more transcription factors, etc.] the particular genomic element with which the likelihood value is associated; (c) determining, by the processor, a predicted change to one or more splicing events using the first plurality of likelihood values and the second plurality of likelihood values [e.g., determining, by the processor, values of one or more difference metrics quantifying a change between at least a portion of the first and second pluralities of likelihood values]; and (f) storing, by the processor, data representing the predicted change(s) to one or more splicing events and/or providing, by the processor, data representing the predicted change(s) to one or more splicing events for display, and/or further processing.

In certain embodiments, provided methods comprise identifying, by the processor, one or more neoantigen and/or neoepitope candidates based at least in part on the second nucleotide sequence data and the data representing the predicted change(s) to one or more splicing events [e.g., selecting, by the processor, one or more mutations of the second nucleotide sequence, relative to the first nucleotides sequence, for inclusion in a pharmaceutical composition comprising a polynucleotide comprising at least a portion of the first nucleotide sequence with the one or more mutations].

In some aspects, the present disclosure provides systems (e.g., for predicting isoforms (e.g., RNA isoforms, protein isoforms, peptide isoforms) due to alternative splicing events from nucleotide sequence data), the system comprising: a processor of a computing device and memory with instructions stored thereon, wherein the instructions, when executed by the processor, cause the processor to: (a) receive nucleotide sequence data representing a sequence of a plurality of nucleotides; (b) determine, using a machine learning model and based on the nucleotide sequence data, a plurality of likelihood values, wherein each likelihood value is associated with (i) a particular nucleotide of the sequence and (ii) a particular one of the one or more genomic element(s), and wherein each likelihood value represents and/or quantifies a likelihood that the particular nucleotide is part of [e.g., is part of a subsequence (plurality of nucleotides) that functions as, for example by coding for a particular protein, RNA, binding one or more transcription factors, etc.] the particular genomic element with which the likelihood value is associated; (c) determine, for at least a portion of the nucleotide sequence data, two or more predicted isoforms (e.g., predicted to result from alternative splicing) using the plurality of likelihood values; and (d) store data representing the two or more predicted isoforms and/or provide data representing the two or more predicted isoforms for display, and/or further processing.

In certain embodiments, instructions cause the processor to identify one or more neoantigen and/or neoepitope candidates based at least in part on the data representing the two or more predicted isoforms [e.g., selecting, by the processor, at least a portion of one of the two or more predicted isoforms for inclusion a pharmaceutical composition].

In some aspects, the present disclosure provides systems (e.g., for predicting impact of one or mutations to a nucleotide sequence on alternative splicing events), the system comprising: a processor of a computing device; and memory having instructions stored thereon, wherein the instructions, when executed by the processor, cause the processor to: (a) receive first nucleotide sequence data representing a first sequence of a plurality of nucleotides; (b) determine, using a machine learning model and based on the first nucleotide sequence data, a first plurality of likelihood values, wherein each likelihood value of the first plurality of likelihood values is associated with (i) a particular nucleotide of the first sequence and (ii) a particular one of the one or more genomic element(s), and wherein each likelihood value represents and/or quantifies a likelihood that the particular nucleotide is part of [e.g., is part of a subsequence (plurality of nucleotides) that functions as, for example by coding for a particular protein, RNA, binding one or more transcription factors, etc.] the particular genomic element with which the likelihood value is associated; (c) receive second nucleotide sequence data representing a second nucleotide sequence corresponding to a mutated version of the first nucleotide sequence [e.g., an identification (e.g., a list) of mutations to the first nucleotide sequence; e.g., second sequence data representing the second, mutated, nucleotide sequence]; (d) determine, using the machine learning model and based on the second nucleotide sequence data, a second plurality of likelihood values, wherein each likelihood value of the second plurality of likelihood values is associated with (i) a particular nucleotide of the second sequence and (ii) a particular one of the one or more genomic element(s), and wherein each likelihood value represents and/or quantifies a likelihood that the particular nucleotide is part of [e.g., is part of a subsequence (plurality of nucleotides) that functions as, for example by coding for a particular protein, RNA, binding one or more transcription factors, etc.] the particular genomic element with which the likelihood value is associated; (c) determine a predicted change to one or more splicing events using the first plurality of likelihood values and the second plurality of likelihood values [e.g., determining, by the processor, values of one or more difference metrics quantifying a change between at least a portion of the first and second pluralities of likelihood values]; and (f) store data representing the predicted change(s) to one or more splicing events and/or provide data representing the predicted change(s) to one or more splicing events for display, and/or further processing.

In certain embodiments, instructions cause the processor to identify one or more neoantigen and/or neoepitope candidates based at least in part on the second nucleotide sequence data and the data representing the predicted change(s) to one or more splicing events [e.g., selecting, by the processor, one or more mutations of the second nucleotide sequence, relative to the first nucleotides sequence, for inclusion in a pharmaceutical composition comprising a polynucleotide comprising at least a portion of the first nucleotide sequence with the one or more mutations].

In some aspects, the present disclosure provides a pharmaceutical composition comprising a polyribonucleotide encoding (e.g., a vaccine construct comprising) a plurality of neoepitopes, wherein at least a portion of the plurality of neoepitopes are neoepitope candidates identified by the methods or systems of the present disclosure (e.g., as described in paragraphs above). Features of embodiments described with respect to one aspect of the invention may be applied with respect to another aspect of the invention.

### BRIEF DESCRIPTION OF THE DRAWING

The foregoing and other objects, aspects, features, and advantages of the present disclosure will become more apparent and better understood by referring to the following description taken in conjunction with the accompanying drawings, in which:

FIG. 1 is a block flow diagram showing an example process for detecting and/or localizing of one or more genomic elements within a nucleotide sequence, according to an illustrative embodiment.

FIG. 2 is a schematic showing an example implementation of a genomic element segmentation technology, in accordance with certain embodiments described herein.

FIG. 3 is a set of illustrative graphs showing various channels of likelihood values and how they can be used to assign genomic element labels to nucleotides and/or tokens in a nucleotide sequence, according to certain embodiments.

FIG. 4A is a schematic showing an example machine learning model architecture comprising a language model (LM) encoder and a segmentation head, according to an illustrative embodiment.

FIG. 4B is a schematic illustrating example approaches for training and extracting embedding vectors from a LM, according to an illustrative embodiment.

FIG. 4C is a block flow diagram showing an example process for detecting and/or localizing of one or more genomic elements within a nucleotide sequence according to an illustrative embodiment.

FIG. 4D is a schematic illustrating example approaches for utilizing encoder models that incorporate one or more convolutional layers, according to an illustrative embodiment.

FIG. 4E is a block flow diagram of an example process for predicting isoforms (e.g., RNA isoforms, protein isoforms, peptide isoforms) due to alternative splicing events from nucleotide sequence data, according to an illustrative embodiment.

FIG. 4F is a block flow diagram of an example process for predicting impact of one or mutations to a nucleotide sequence on alternative splicing events, according to an illustrative embodiment.

FIG. 5A is a schematic showing an example system for use in implementing certain machine learning models and related processes described herein.

FIG. 5B is a schematic showing example non-transitory storage medium for use in storing and/or implementing certain machine learning models and related processes described herein.

FIG. 6 is a block diagram of an exemplary cloud computing environment, used in certain embodiments.

FIG. 7 is a block diagram of an example computing device and an example mobile computing device used in certain embodiments.

FIG. 8A is a diagram showing an exemplary embodiment of a machine learning model comprising a language model-based encoder and a segmentation head and receiving a nucleotide sequence as an input and determining a plurality of probabilities as an output at single nucleotide resolution.

FIG. 8B is diagram showing an exemplary embodiment of a segmentation head using a 1D U-Net architecture with 2 downsampling and 2 upsampling convolutional blocks with matched U-net connections.

FIG. 8C is a bar chart showing a performance measure calculated as Matthews correction coefficient (MCC) of an exemplary embodiment of a machine learning model trained on nucleotide sequences with 3 kb and 10 kb lengths for annotating 14 genomic elements.

FIG. 8D is a set of plots showing an exemplary annotation and probabilities related to 14 genomic elements produced by a machine learning model for NOP56/IDH3B gene locus.

FIG. 8E is a bar chart showing MCC performance for exemplary embodiments of machine learning models of various architectures for annotating 14 genomic elements.

FIG. 8F is a bar chart showing average MCC performance across annotating 14 genomic elements for exemplary embodiments of machine learning models of various architectures.

FIG. 9A is bar chart showing percentages of nucleotide sequences of 10 kb length containing various genomic elements in training and testing sets used for machine learning models.

FIG. 9B is bar chart showing percentages of nucleotides in nucleotide sequences of 10 kb length containing various genomic elements in training and testing sets used for machine learning models.

FIG. 10A is a bar chart showing precision-recall area under the curve (PR-AUC) performance for various models for annotating tissue-invariant promoters.

FIG. 10B is a bar chart showing MCC performance for exemplary embodiments of various models for tissue-specific enhancers.

FIG. 10C is a bar chart showing inference times for various models to annotate a 10 kb nucleotide sequence.

FIG. 11A is a bar chart showing MCC performance for exemplary embodiments of machine learning models trained on sequences with 3 kb, 10 kb, 20 kb, and 30 kb lengths to annotate 14 genomic elements.

FIG. 11B is a bar chart showing average MCC performance across annotating 14 genomic elements for exemplary embodiments of machine learning models trained on sequences with 3 kb, 10 kb, 20 kb, and 30 kb lengths.

FIG. 11C is a plot showing average MCC performance across annotating 14 genomic elements for exemplary embodiments of machine learning models trained on sequences with 10 kb lengths with and without context-length rescaling per input sequence length.

FIG. 11D is a plot showing average MCC performance across annotating 14 genomic elements for exemplary embodiments of machine learning models trained on sequences with 3 kb, 10 kb, 20 kb, and 30 kb lengths with context-length rescaling per input sequence length.

FIG. 11E is a set of plots showing an exemplary annotation and probabilities related to 14 genomic elements as obtained by a machine learning model for a 50 kb region at the TMEM230/PCNA/CDS2 gene locus.

FIG. 12A is a plot showing average MCC performance across annotating 14 genomic elements for exemplary embodiments of machine learning models trained on sequences with 10 kb lengths with and without context-length rescaling per input sequence length.

FIG. 12B is a bar chart showing MCC performance for annotating 14 genomic elements in nucleotide sequences of 20 kb length for exemplary embodiments of machine learning models trained on sequences with 10 kb lengths with and without context-length rescaling per input sequence length.

FIG. 12C is a bar chart showing MCC performance for annotating 14 genomic elements in nucleotide sequences of 50 kb length for exemplary embodiments of machine learning models trained on sequences with 10 kb lengths with and without context-length rescaling per input sequence length.

FIG. 12D is a bar chart showing MCC performance for annotating 14 genomic elements in nucleotide sequences of 100 kb length for exemplary embodiments of machine learning models trained on sequences with 10 kb lengths with and without context-length rescaling per input sequence length.

FIG. 13A is a set of plots showing an exemplary annotation and probabilities related to exon, intron, splice acceptor, and splice donor for two exemplary embodiments of machine learning models at the EBF4 gene locus.

FIG. 13B is a bar chart showing PR-AUC for two exemplary embodiments of machine learning models for splice acceptor and splice donor annotation.

FIG. 13C is a bar chart showing MCC performance for two exemplary embodiments of machine learning models for splice acceptor, splice donor, exon, and intron annotations for all regions of a whole chromosome.

FIG. 13D is a bar chart showing MCC performance for two exemplary embodiments of machine learning models for splice acceptor, splice donor, exon, and intron annotations for regions containing genes only in the positive strand of a whole chromosome.

FIG. 13E is a bar chart showing MCC performance for two exemplary embodiments of machine learning models for splice acceptor, splice donor, exon, and intron annotations for regions containing genes only in the negative strand of a whole chromosome.

FIG. 14A is a bar chart showing PR-AUC for two exemplary embodiments of machine learning models for splice acceptor and splice donor annotation.

FIG. 14B is a bar chart showing MCC performance for two exemplary embodiments of machine learning models for splice acceptor, splice donor, exon, and intron annotations for all regions of a whole chromosome.

FIG. 14C is a bar chart showing MCC performance for two exemplary embodiments of machine learning models for splice acceptor, splice donor, exon, and intron annotations for regions containing genes only in the positive strand of a whole chromosome.

FIG. 14D is a bar chart showing MCC performance for two exemplary embodiments of machine learning models for splice acceptor, splice donor, exon, and intron annotations for regions containing genes only in the negative strand of a whole chromosome.

FIG. 15A is a diagram of RON exon 11 wildtype minigene with exons and introns and a set of plots showing probability values of various genomic elements in the minigene determined by a machine learning model.

FIG. 15B is two plots showing probability values of various genomic elements in RON exon 11 minigene with various mutations marked by black stars determined by a machine learning model and a bar chart showing percentage differences for various genomic elements between the mutated and a wildtype minigene.

FIG. 15C is two plots showing probability values of various genomic elements in RON exon 11 minigene with various mutations marked by black stars determined by a machine learning model and a bar chart showing percentage differences for various genomic elements between the mutated and a wildtype minigene.

FIG. 15D is a scatter plot showing exon prediction determined by a machine learning model per percentage of RON exon 11 (AE) inclusion for different minigene variants with mutations.

FIG. 16A is a diagram showing zero-shot and few-shot generalization training procedures for exemplary embodiments of machine learning models.

FIG. 16B is a grid plot showing MCC performance for an exemplary embodiment of a machine learning model trained on human nucleotide sequences for annotating various genomic elements in nucleotide sequences of different species.

FIG. 16C is a plot showing average MCC performance for an exemplary embodiment of a machine learning model for annotating various genomic elements per divergence time in millions of years ago (MYA).

FIG. 16D is a radar plot showing MCC performance for exemplary embodiments for two machine learning models, one trained on only human nucleotide sequences and second trained on nucleotide sequence of multiple species, for annotating various genomic elements in nucleotide sequences of a training set of the nucleotide sequence of multiple species.

FIG. 16E is a radar plot showing MCC performance for exemplary embodiments for two machine learning models, one trained on only human nucleotide sequences and second trained on nucleotide sequence of multiple species, for annotating various genomic elements in nucleotide sequences of human-close species.

FIG. 16F is a radar plot showing MCC performance for exemplary embodiments for two machine learning models, one trained on only human nucleotide sequences and second trained on nucleotide sequence of multiple species, for annotating various genomic elements in nucleotide sequences of human-distant species.

FIG. 16G is a set of grid plots showing MCC performance for exemplary embodiments for two machine learning models, one trained on only human nucleotide sequences and second trained on nucleotide sequence of multiple species, for annotating various genomic elements in nucleotide sequences of 4 species.

FIG. 17A is a set of grid plots showing MCC performance for exemplary embodiments for two machine learning models, one trained on only human nucleotide sequences and second trained on nucleotide sequence of multiple species, for annotating various genomic elements in the nucleotide sequences of multiple species.

FIG. 17B is a set of grid plots showing MCC performance for exemplary embodiments for two machine learning models, one trained on only human nucleotide sequences and second trained on nucleotide sequence of multiple species, for annotating various genomic elements in nucleotide sequences of multiple species.

FIG. 17C is a set of grid plots showing MCC performance for exemplary embodiments for two machine learning models, one trained on only human nucleotide sequences and second trained on nucleotide sequence of multiple species, for annotating various genomic elements in nucleotide sequences of multiple species.

FIG. 18 is a bar graph showing area under precision recall curves for predictions of splice alterations from cancer data.

FIG. 19A is a schematic showing an example Nucleotide Transformer (NT)-based genomic element segmentation model, according to an illustrative embodiment.

FIG. 19B is a schematic showing an example genomic element segmentation model that uses an Enformer model as an encoder, according to an illustrative embodiment.

FIG. 19C is a schematic showing an example genomic element segmentation model that uses a Borzoi model as an encoder, according to an illustrative embodiment.

FIG. 20 is a set of plots comparing performance of the three different models shown in FIGS. 20A-C, according to an illustrative embodiment.

FIG. 21 is a set of plots showing performance of an example genomic element segmentation model, according to an illustrative embodiment.

FIG. 22 is a set of plots showing performance of an example genomic element segmentation model in comparison with certain baselines, according to an illustrative embodiment.

FIG. 23 is a set of plots showing performance of an example genomic element segmentation model on various species, according to an illustrative embodiment.

FIG. 24 is a set of plots showing performance of an example genomic element segmentation model, according to an illustrative embodiment.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Definitions

About or Approximately: The term “about” or “approximately”, when used herein in reference to a value, refers to a value that is similar to the referenced value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Biological sequence: As used herein, the term “biological sequence” refers to a physical sequence of biological building blocks (e.g., nucleotides, amino acids, etc.) typically forming a biopolymer, such as DNA, RNA, and polypeptides (e.g., proteins and/or peptides). In certain embodiments, a biological sequence is or comprises a nucleotide sequence. For example, a biological sequence may be a DNA sequence. For example, a biological sequence may be an RNA sequence. In certain embodiments, a biological sequence may be a sequence of amino acids, such as a polypeptide sequence (e.g., a protein sequence; e.g., a peptide sequence).

Cancer: The term “cancer” is used herein to generally refer to a disease or condition in which cells of a tissue of interest exhibit relatively abnormal, uncontrolled, and/or autonomous growth, so that they exhibit an aberrant growth phenotype characterized by a significant loss of control of cell proliferation. In some embodiments, cancer may comprise cells that are precancerous (e.g., benign), malignant, pre-metastatic, metastatic, and/or non-metastatic. In some embodiments, cancer may be characterized by a solid tumor. In some embodiments, cancer may be characterized by a hematologic tumor. In general, examples of different types of cancers known in the art include, for example, triple negative breast cancer (TNBC), hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, ovarian cancer, breast cancer, glioblastomas, colorectal cancer, gastro-intestinal cancers and nervous system cancers, benign lesions such as papillomas, and the like.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of one or more named elements or steps” also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: In some embodiments, the methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Machine learning module, machine learning model: As used herein, the terms “machine learning module” and “machine learning model” are used interchangeably and refer to a computer implemented process (e.g., a software function) that implements one or more particular machine learning algorithms, such as an artificial neural networks (ANNs), random forest, decision trees, support vector machines, and the like, in order to determine, for a given input, one or more output values. In certain embodiments, machine learning models are deep learning models or deep neural networks—for example, ANNs that comprise, in addition to an input layer and an output layer, one or more hidden layers (e.g., in between). Examples of deep learning models include, without limitation, recurrent neural networks (RNNs) (e.g., long short-term memory networks (LSTMs), bi-directional LSTMs (biLSTMs)), attention-based networks, such as transformer models, and convolutional neural networks (CNNs). In some embodiments, machine learning modules implementing machine learning techniques are trained in a supervised manner, for example using curated and/or manually annotated datasets. In certain embodiments, machine learning models may be trained in an unsupervised manner, using unlabeled data. In certain embodiments, a machine learning model may be trained via a reinforcement approach, for example wherein a reward/penalty system is used to train a machine learning model to learn strategies for accomplishing specified tasks. Training a machine learning model may be used to determine various parameters of a model, such as weights associated with layers in neural networks. In some embodiments, once a machine learning module is trained, e.g., to accomplish a specific task, such as predicting types of hidden nucleotides within of nucleotide sequences (e.g., DNA sequences) based on their context, values of determined parameters are fixed and the machine learning module is used to process new data (e.g., different from the training data), such as a new nucleotide sequence. The process of presenting a machine learning model with multiple examples, comparing its output to known, ground truth values, and updating parameters to progressively improve performance may be referred to as training, while the use of a (e.g., previously trained) machine learning model to generate predictions about new data, for which ground truth values may be unknown, may be referred to as inference. In some embodiments, machine learning modules may receive feedback, e.g., based on user review of accuracy, and such feedback may be used as additional training data, for example to dynamically update the machine learning module. In some embodiments, a trained machine learning module is a classification algorithm with adjustable and/or fixed (e.g., locked) parameters, e.g., a random forest classifier. In some embodiments, two or more machine learning modules may be combined and implemented as a single module and/or a single software application. In some embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC), field programmable gate arrays (FPGAs), and the like).

Gene and gene element(s): The term “gene”, as used herein, refers to a series of nucleotides in a DNA sequence that is transcribed into a functional RNA (e.g., encoding a specific protein and/or portions thereof). The term “gene elements”, as used herein, refers to those portions of nucleotide sequences that are synthesized to create proteins and/or portions thereof (e.g., protein segments). In certain embodiments, gene elements contrast with regulatory elements that do not code for proteins, but, rather, are collections of nucleotides that, for example, impact expression of genes. Gene elements include, without limitation, protein-coding genes, lncRNAs, 5′UTRs, 3′UTRs, exons, introns, splice acceptors and donor sites.

Genomic element(s): As used herein, the term “genomic elements” refers to subunits of nucleotide sequences, which may be known, determined, or predicted to perform particular functions, such as coding for proteins and/or controlling gene expression. Genomic elements include, for example, gene elements and regulatory elements.

Nucleic acid: As used herein, the term “nucleic acid” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleotides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context).

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attaching to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 35 or more amino acids; in some embodiments, a conserved region encompasses at least one stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous amino acids. In some embodiments, a relevant polypeptide may comprise or consist of a fragment of a parent polypeptide.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is an mRNA. In some embodiments where an RNA is an mRNA, an RNA typically comprises at its 3′ end a poly(A) region. In some embodiments where an RNA is an mRNA, an RNA typically comprises at its 5′ end an art-recognized cap structure, e.g., for recognizing and attachment of an mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. One of ordinary skill in the art will understand that when a single-stranded RNA is referred to as “encoding,” it can mean that it comprises a nucleic acid sequence that itself encodes or that it comprises a complement of the nucleic acid sequence that encodes. In some embodiments, a single-stranded RNA can be a self-amplifying RNA (also known as self-replicating RNA).

Regulatory element(s): The term “regulatory elements”, as used herein, refer to portions of nucleotide sequences (e.g., non-coding portions) that regulate gene expression (e.g., transcriptional regulation, post-transcriptional regulation, translational regulation, or post-translational regulation). Regulatory elements include, without limitation, poly(A) signal sequences, promoters (e.g., tissue-invariant promoters, or tissue-specific promoters), enhancers (e.g., tissue-invariant enhancers or tissue-specific enhancers), and CTCF-binding sites.

Sequence data: The term “sequence data”, as used herein, refers to a (e.g., computer) representation of a biological sequence. Sequence data may represent structural components, or building blocks, of a biological sequence in a variety of forms, such as a series of alpha numeric characters, a series of tokens, a set of one-hot encodings, and the like. In certain embodiments, orders of characters, tokens, one-hot vectors etc., may encode and/or reflect relative positions of corresponding sub-units within a biological sequence. For example, in certain embodiments, nucleotide sequence data represents a nucleotide sequence, such as a polynucleotide or DNA sequence. In certain embodiments, polypeptide sequence data represents a polypeptide (e.g., protein) sequence.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

### A. Nucleotide Sequences and Genomic Elements

A.i. Biological Sequences

In certain embodiments, systems and methods of the present disclosure are used to analyze biological sequences, such as nucleotide sequences, to identify and localize—e.g., segment—various genomic elements therein. A nucleotide sequence may represent genetic material of an organism (e.g., using biomolecules, biochemical compounds), and comprise a sequence of nucleotides, as in, for example, a nucleic acid sequence, such as a deoxyribonucleic acid (DNA) sequence or a ribonucleic acid (RNA) sequence.

In certain embodiments, a DNA sequence may be derived from another type of biological sequence, such as an RNA and/or polypeptide sequence. Said another way, in certain embodiments, certain (e.g., higher level) biological sequences, such as RNA and/or polypeptide sequences, can be used to determine a corresponding DNA sequence. For example, corresponding DNA and coding sequences can be determined based on RNA and protein sequences, respectively.

A.ii. Genomic Elements

As described in further detail herein, systems and methods of the present disclosure provide technologies for identifying and localizing various genomic elements within nucleotide sequences, such as DNA sequences. In certain embodiments, genomic elements include one or more portions of a DNA sequence (e.g., consecutive nucleic acids) that carry out one or more particular functions. In certain embodiments, genomic elements include gene elements as well as regulatory elements. For example, gene elements may include protein-coding regions, introns, exons, etc. For example, regulatory elements may include elements such as transcriptional control elements, translational control elements, and post-translational control elements. Regulatory elements may offer various types of control, such as positive control (e.g., turning on gene expression), negative control (e.g., turning off gene expression), and co-regulation (e.g., turning two or more genes on, together).

In certain embodiments, a genomic element is associated with (e.g., identifies) one or more specific parts of a nucleotide sequence. Genomic elements may be associated with (e.g., may determine, may be excluded from) transcriptional processes (e.g., transcribing a segment of DNA into RNA, synthesizing a messenger RNA (mRNA)). Genomic elements may be associated with (e.g., determine, are excluded from determining) post-transcriptional processes (e.g., of RNA). Genomic elements may be associated with (e.g., determine, are excluded from determining) translational processes (e.g., producing a polypeptide, protein, or peptide from an RNA). Genomic elements may be associated with (e.g., determine, are excluded from determining) post-translational modifications (e.g., of proteins). Genomic elements may be associated with (e.g., may determine, e.g., may be excluded from determining) protein properties (e.g., conformation, structure).

In certain embodiments, genomic elements comprise one or more genetic features of eukaryotes. In certain embodiments, genomic elements comprise one or more genetic features of prokaryotes.

In certain embodiments, genomic elements comprise one or more parts of a nucleotide sequence that are associated with (e.g., determine) encoding a protein/polypeptide/peptide amino acid sequence. Genomic elements may comprise one or more protein-encoding genes, at least a part of a protein-encoding gene, one or more coding regions of a gene, or one or more constrained coding regions. Genomic elements may comprise at least a part of an exon. Genomic elements may comprise at least a part of an intron. Genomic elements may comprise at least a part of a splice donor site. Genomic elements may comprise at least a part of a splice acceptor site.

In certain embodiments, genomic elements include one or more regions of a nucleotide sequence (e.g., subsequences of nucleotides) that are not translated into a protein. For example, certain portions of DNA sequences may be transcribed into non-coding RNAs. Accordingly, in certain embodiments, a genomic element may include a non-coding RNA region—that is a portion of a DNA sequence corresponding to (e.g., that would be transcribed to) a non-coding RNA. In certain embodiments, genomic elements may be DNA regions that correspond to particular types of non-coding RNAs, such as short non-coding RNA, microRNAs (miRNAs), small interfering RNAs (siRNAs), piwi-interacting RNAs (piRNAs), small nucleolar RNAs (snoRNAs)). Genomic elements may comprise long (e.g., longer than 200 nucleotides) non-coding RNAs (e.g., intergenic lincRNAs, intronic ncRNAs, and sense and antisense lncRNAs).

In certain embodiments, genomic elements may comprise one or more nucleotide sequences that determine the stability and/or expression of a gene transcript. Genomic elements may comprise a 5′ untranslated region (5′UTR). Genomic elements may comprise a 3′ untranslated region (3′UTR). A 3′ UTR may comprise one or more regulatory regions that post-transcriptionally influence gene expression.

In certain embodiments, genomic elements comprise one or more parts of a nucleotide sequence associated with transcript modification processes (e.g., capping, decapping, cleavage, splicing, addition, removal, elongation) of the sequence. For example, certain genomic elements, such as splicers, may indicate positions in a nucleotide sequence for splicing. Genomic elements may comprise one or more parts of a nucleotide sequence associated with polyadenylation. Genomic elements may comprise at least a part of a polyadenylation (polyA) signal sequence.

In certain embodiments, genomic elements comprise one or more parts of a nucleotide sequence that regulate (e.g., enhance a rate of, decrease a rate of, mediate, initiate, terminate) transcription of an mRNA or translation of a protein, polypeptide, or peptide. For example, certain genomic elements, such as a particular enhancer, may enhance a transcription rate of a particular gene. Genomic elements may comprise at least a part of an enhancer (e.g., tissue-invariant enhancers, tissue-specific enhancers, developmental enhancers, Hk enhancers). Genomic elements may comprise at least a part of a promoter (e.g., tissue-invariant promoter, tissue-specific promoter, TATA box-containing promoter, non-TATA box-containing promoter). Genomic elements may comprise at least a part of CCCTC-binding factor (e.g., transcriptional repressor CTCF, 11-zinc finger protein)-binding sequence. Genomic elements may comprise at least a part of a transcription factor binding site.

In certain embodiments, genomic elements comprise one or more parts of a nucleotide sequence that determine its 3D structure (e.g., primary, secondary, tertiary, quaternary). For example, certain genomic elements, such as histones, affect quaternary structure of a DNA sequence. Genomic elements may comprise at least a part of a chromatin (e.g., open chromatin), at least a part of a histone-binding sequence, elements related to protein stability, elements related to determining polynucleotide melting temperature, and/or elements related to determining protein melting temperature.

a.iii. Biological Significance of Nucleotide sequence Annotations

In certain embodiments, annotating nucleotide sequences to identify various genomic elements as described herein can be used to facilitate downstream nucleotide sequence analysis, for example, relevant for evaluating impact of various genes, mutations, and the like. For example, in certain embodiments, one or more genetic mutations may cause dysregulation during various genetic processes (e.g., transcription, translation). These dysregulations and mutations may further contribute to the development of various diseases, including cancer. Significant effort is focused on characterizing effects (e.g., mechanisms, impact) of such mutations and dysregulations as well as development of associated therapies (e.g., therapeutics). One of the central steps in mutation characterization is the ability to annotate a nucleotide sequence to identify which genomic elements are affected by mutations. Multiple studies suggest that mutations occurring at various stages of using genetic information by living organisms (e.g., DNA transcription, post-transcription, RNA translation, post-translation) are related to various diseases and dysfunctions, including cancer. To understand an impact of a particular mutation and to develop potential therapies, one requires to understand which genomic element a particular mutation affects—an annotating task. To this end, the obtained annotated nucleotide sequence data can be used to reveal insight about encoded information and, in particular, provide representation of the genomic elements for downstream tasks of understanding the underlying biological processes and developing potential therapies. Development and validation of a therapeutic often requires extensive in vitro and in vivo testing, and clinical trial studies. These processes are often time-consuming and expensive. In contrast, the present disclosure operates on an insight that mutation effects can be studied by annotating and evaluating genomic data in silico. Such an approach may result in significant time and cost savings.

For example, lncRNAs are associated with animal neurodevelopment, cell cycle regulation, cell regulation, tumorigenesis, and metastasis (Diederichs S, et al. Eur Urol. 2014; 65 (6): 1152-3; Shi X, et al. Cancer Lett. 2013:339 (2): 159-166). Moreover, various diseases and cancers are associated with mutations and dysregulations of lncRNAs (Wapinski O, Chang H Y. Trends Cell Biol. 2011; 21 (6): 354-361; Zeng M, et al. Methods. 2020; 179:73-80; 5. Zeng M, et al. Brief Bioinform. 2021; 23 (1): bbab360; Zeng M, et al. IEEE/ACM Trans Comput Biol Bioinforma. 2021; 18 (6): 2353-2363). For example, multiple evidence suggest that mutations within 5′ UTRs are often linked with diseases, including cancer (N. Ryczek et al. Int J Mol Sci. 2023 February; 24 (3): 2976; Mularoni L., et al. Genome Biol. 2016; 17:128. doi: 10.1186/s13059-016-0994-0). For example, 3′ UTRs contribute to the regulation of mRNA stability and translation, while dysregulation of a 3′ UTR and/or 3′ UTR elements, that modulate 3′ UTR interactions with RNA-binding proteins (RBPs) and/or microRNAs (miRNAs), can be associated with cancer and contribute to pathogenesis (Schuster et al., 2023, Cell Reports 42, 112840). For example, mutations in splicing sequences may lead to a disruption of splicing processes leading to both germline and somatic diseases (e.g., cystic fibrosis, Duchenne muscular dystrophy, and cancer (E. Flemington et al., Nucleic Acids Res. 2023 Apr. 24; 51 (7): e42)). For example, Poly(A) signal sequences modulate mRNA stability, translation, and subcellular localization (Proudfoot N. J. Genes Dev. 2011; 25 (17): 1770-1782). Mutations in Poly(A) signal sequences may play a key role in the occurrence and development of various diseases (Di Giammartino D. C., et al. Mol Cell. 2011; 43 (6): 853-866; Mitra M., Johnson E. L. Genome Biol. 2018; 19 (1)). For example, various promoters and their mutations are often linked to cancer (e.g., glioblastoma is related to TERT promoter (Lee et al. Cancer Res Treat. 2022 January; 54 (1): 75-83); and colorectal cancer to ETV1 promoter (Orlando G., et al. Nat Genet. 2018 October; 50 (10): 1375-1380)).

In certain embodiments, genetic variations (e.g., polymorphisms, mutations) lay at a root of multiple genetic diseases, such as cancer. Identifying and localizing such genetic variations may lead to detection and/or prognosis of various diseases. For example, various genomic elements may be identified and localized for a reference nucleotide sequence and one or more genetic variants (e.g., nucleotide sequences with mutations, polymorphisms). A reference nucleotide sequence may be a wild-type nucleotide sequence. By comparing the resulting annotations, impact of sequence variants on genomic elements present in the genomic sequence may be evaluated. For example, the ability to identify a set of genomic elements (e.g., identified with specific likelihood values, within likelihood ranges) (e.g., and/or a presence of a set of mutations) in specific parts of a nucleotide sequence may allow them to be correlated with (e.g., an onset of, a presence of, a progression of) a specific disease. Additionally or alternatively, a presence of a set of mutations within specific parts of a nucleotide sequence [e.g., identified as belonging to a set of genomic elements (e.g., identified with a particular likelihood or within a range of likelihood values)] may be correlated with (e.g., an onset of, a presence of, a progression of) a specific disease.

### B. Automated Segmentation of Nucleotide Sequences

As described herein, nucleotide sequence segmentation technologies of the present disclosure may be used to determine, for a particular nucleotide sequence, which particular genomic elements (the particular nucleotide sequence) it comprises and where they are located.

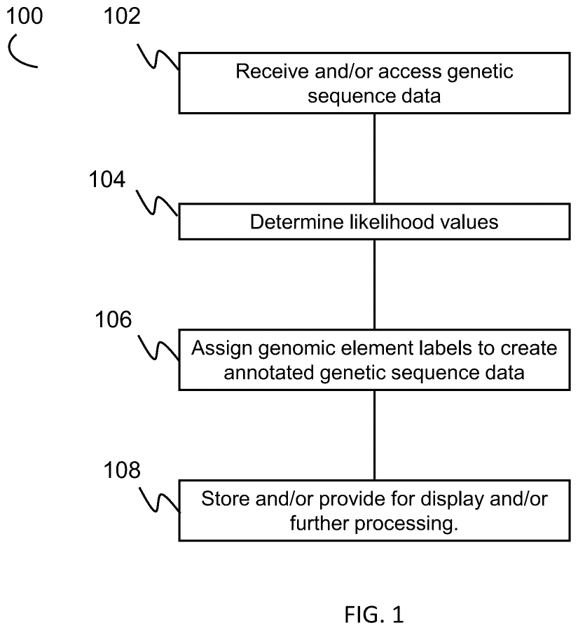

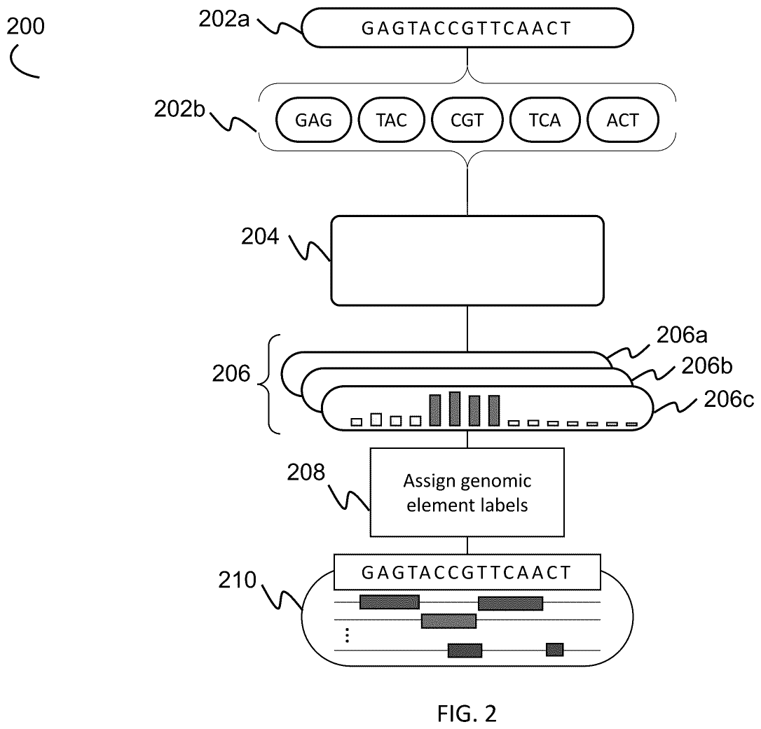

FIGS. 1-2 show an example process 100 and a schematic 200 for segmenting genomic elements within a nucleotide sequence, respectively. As shown in FIGS. 1-2, nucleotide sequence data is received and/or accessed 102, for example, by a processor of a computing device. Nucleotide sequence data 202a may be or comprise a computer representation of one or more nucleotide sequences, such as one or more DNA sequences or portions thereof. Nucleotide sequences may be represented, for example, via a sequence of alphanumeric characters, such as a sequence of letters, each representing a particular nucleotide. For example, a DNA sequence may be represented as a text string with the characters “A”, “C”, “G”, and “T” representing the four naturally occurring nucleotides, adenine, cytosine, guanine, and thymine, respectively. Other manners of representing DNA sequences are also possible. For example, rather than use alphabetical characters, the numbers 1, 2, 3, and 4 may each be assigned to represent a particular naturally occurring base, and a numerical string used to represent a DNA sequence. In certain embodiments, a one-hot encoding approach is used, where each position in a DNA sequence is represented via a four-element vector, populated with zeros and a single one (1) (i.e., a one-hot vector) at a position identifying a particular nucleotide, for example as shown below.

Example one hot-encoding representation of a four-letter DNA sequence alphabet:
Adenine (A): [1 0 0 0]
Cytosine (C): [0 1 0 0]
Guanine (G): [0 0 1 0]
Thymine (T): [0 0 0 1]

In certain embodiments, nucleotide sequence data 202a may be, or be used to generate, a tokenized representation 202b, whereby each non-overlapping set of one or more consecutive nucleotides—e.g., a k-mer (where k is an integer greater than or equal to one)—is represented by a particular token. In certain embodiments, sets of three, four, five, six, etc. of nucleotides are represented by a token.

For example, as shown in FIG. 2, initial nucleotide sequence data 202a may be partitioned into non-overlapping sets of three consecutive nucleic acids, such that a length L sequence is transformed to a tokenized sequence data 202b of length L/3 and, instead of a four-letter alphabet, 4×4×4=64 distinct tokens are available to represent each unique three-nucleic-acid combination.

In certain embodiments, based on nucleotide sequence data 202a and/or 202b, a machine learning model 204 may be used to determine 104 a plurality of likelihood values 206 for a given nucleotide sequence, representing predicted likelihoods of particular nucleotides belonging to various genomic elements. Each likelihood value may be associated with (i) a particular nucleotide or group of nucleotides (e.g., token) of the nucleotide sequence and (ii) a particular genomic element. A particular likelihood value may, accordingly, quantify a likelihood that the particular nucleotide or group of nucleotides belongs to the particular genomic element, as determined by machine learning model 204. Likelihood values may, for example, be floating point numbers between 0 and 1, for example so as to represent a probability that a particular nucleotide or token belongs to a particular genomic element. Likelihood values may be provided on other scales, such as from 0 to 100 (e.g., representing a percentage).

Accordingly, among other things, the present disclosure presents an approach for localizing genomic element in a nucleotide sequence with a single nucleotide resolution-providing a location of the genomic element in the nucleotide sequence. That is, in contrast with approaches that provide a probability of finding a particular genomic element within a particular input sequence (e.g., ‘detection’), approaches of the present disclosure allow for precise locations of genomic elements to be identified, thereby segmenting nucleotide sequences. Among other things, not only does this segmentation approach provide additional, more detailed information, but, additionally or alternatively, may benefit from the knowledge of positional context of the genomic element in the sequence, which, in turn, may lead to improved performance.

Output generated by a machine learning model may take the form of multiple output channels 206a, 206b, 206c (collectively 206), e.g., of likelihood values. Each output channel may correspond to a particular genomic element and may itself comprise a plurality of likelihood values, one for each nucleotide and/or token. Each likelihood value in a particular output channel provides a predicted, e.g., numeric, likelihood that a particular nucleotide or token belongs to the particular genomic element.

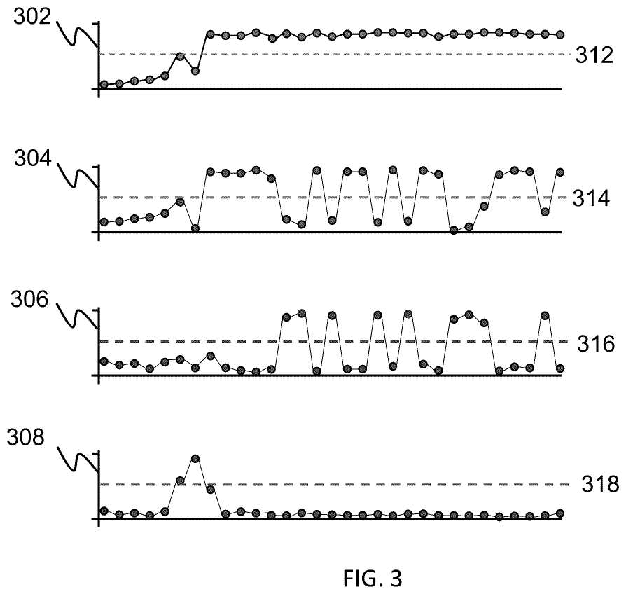

For example, as illustrated in FIG. 3, output channels may include a first channel 302 (e.g., a protein coding gene channel), a second channel 304 (e.g., an intron channel), a third channel 306 (e.g., an exon channel), a fourth channel 308 (e.g., a promotor channel), etc.

In certain embodiments, likelihood values generated via machine learning module may be used to determine and assign genomic element labels to various subsequences of nucleotides. For example, as illustrated in FIG. 3, in certain embodiments, likelihood values may be compared with one or more threshold values 312, 314, 316, 318 to assign genomic element labels. In certain embodiments, a single threshold value is used for all genomic elements and their associated channels of likelihood values. In certain embodiments, multiple (e.g., distinct) threshold values are used.

For example, in certain embodiments, likelihood values in each output channel may be compared with a corresponding threshold value, such that, for example, each nucleotide having a likelihood value for a particular genomic element exceeding a corresponding threshold can be assigned a label for that genomic element. Nucleotide having likelihood values for a particular genomic element lower than the corresponding threshold may not assigned a label for that genomic element. This process can be performed for each genomic element/channel independently. Thresholds can be the same (e.g., a single global threshold) or particular to each genomic element. Thresholds can be determined, e.g., via AUC/ROC analysis, e.g., to achieve a desired specificity/sensitivity. Other criteria may also be used, for example, to identify contiguous sequences, ensuring appropriate length, denoising approaches, avoiding mutually exclusive elements (e.g., introns/exons).

In certain embodiments, additionally or alternatively, other approaches, such as various functions and/or classifiers, may be used. For example, a binary classifier may be used to bin likelihood values into Os and Is, thereby determining positions of a genomic element. Additionally or alternatively, genomic element labels may be assigned based on a combination of likelihood values and their positions in a nucleotide sequence (e.g., a smoothing function). For example, such processing may result in eliminating one or more nucleotides associated with low likelihood values located in between nucleotides associated with high likelihood values as a noise correction. Additionally or alternatively, a set of likelihood values associated with a particular nucleotide may be further processed. For example, using all likelihood values in the set (e.g., finding a particular combination of them), one may determine a set of genomic elements that the nucleotide belongs to. Additionally or alternatively, a set of likelihood values associated with a set of nucleotides (e.g., and/or amino acids) and a set of genomic elements may be further processed (e.g., using a function, relying on a biological insight). For example, a biological insight that genomic elements, such as exons and introns, are mutually exclusive in combination with sets of nucleotides associated with high likelihood values for these genomic elements may help in determining exact locations of these genomic elements. For example, a biological insight that a splicer is located between an exon and an intron in combination with sets of nucleotides associated with high likelihood values for exons and introns may help in determining exact locations of splicers.

### C. Machine Learning Models for Genomic Element Segmentation

Machine learning models used in connection with technologies of the present disclosure (for example, to generate likelihood values for localizing genomic elements within DNA sequences as described herein) may utilize and implement various machine learning techniques. For example, a machine learning model may be a deep learning model (e.g., an artificial neural network with one or more, e.g., plurality of, hidden layers). Example deep learning models used herein include, among others, language models (LMs) and convolutional neural networks (CNNs).

C.i. Genomic Language Models

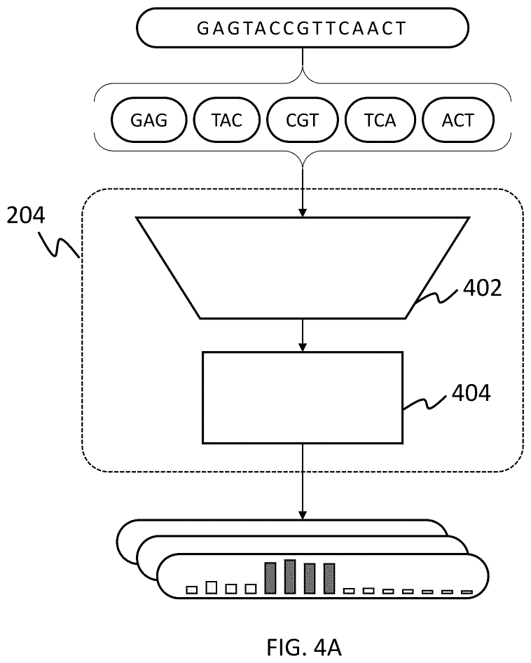

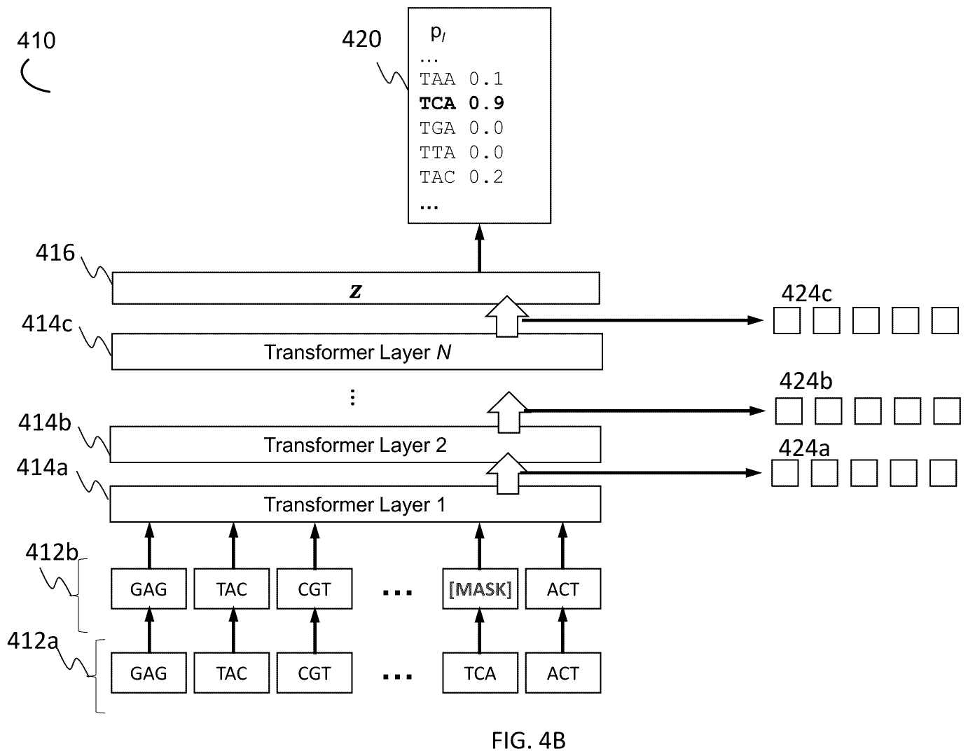

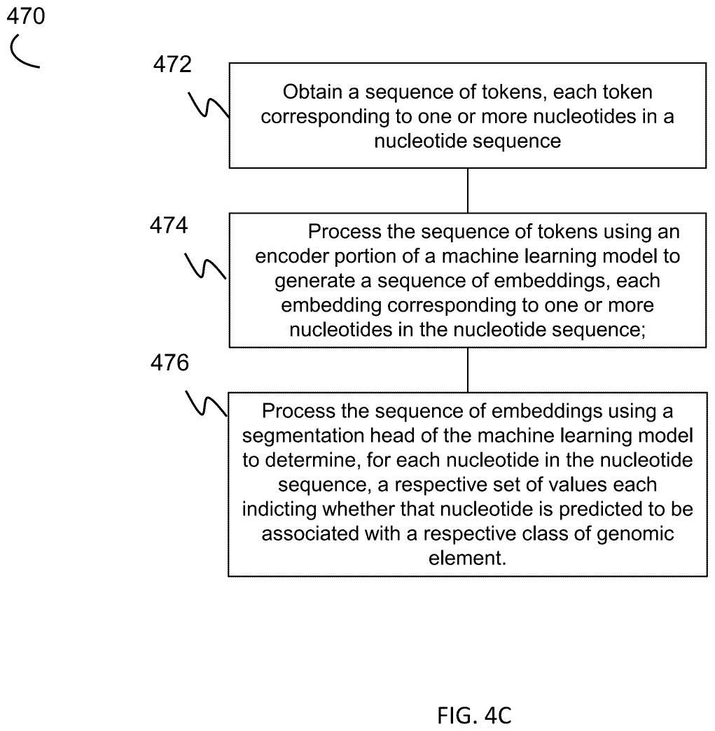

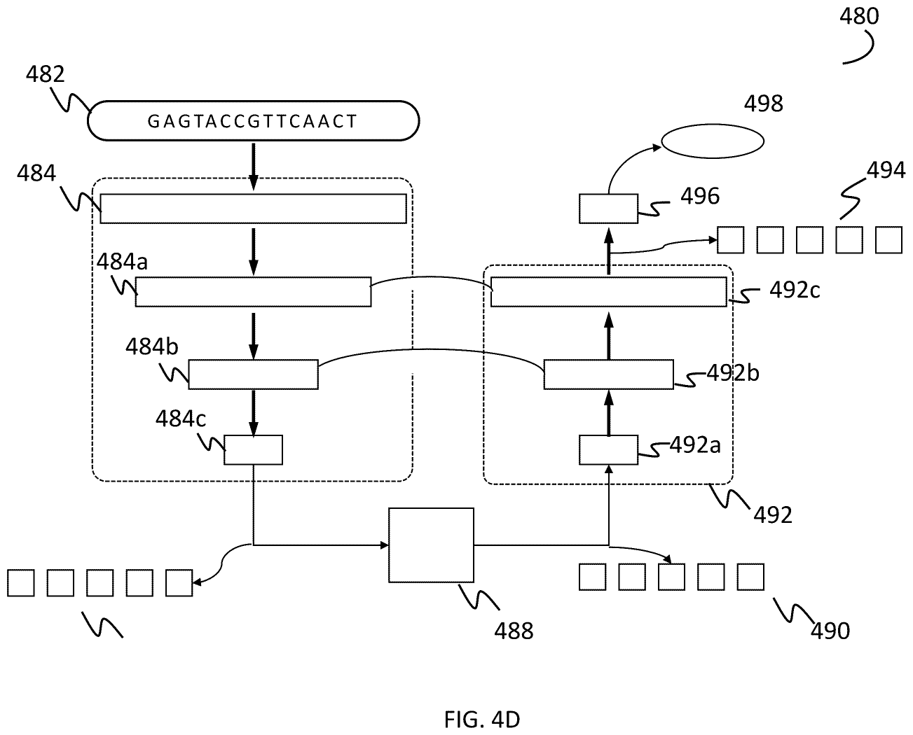

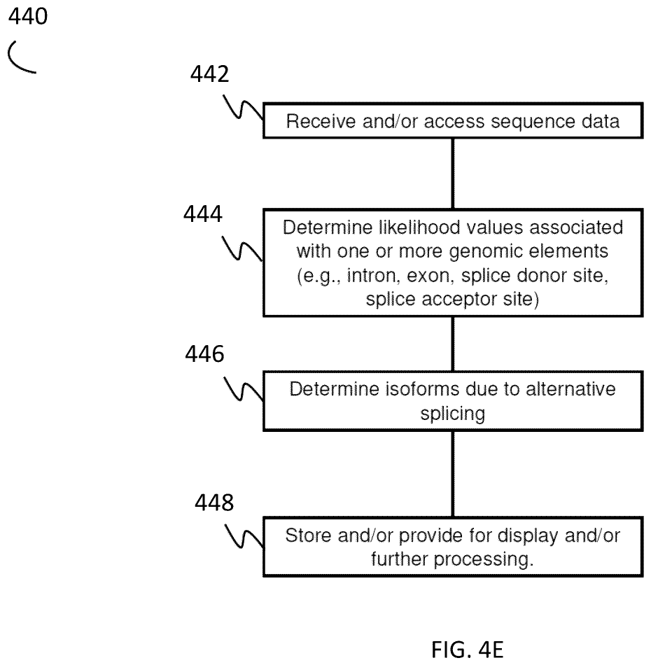

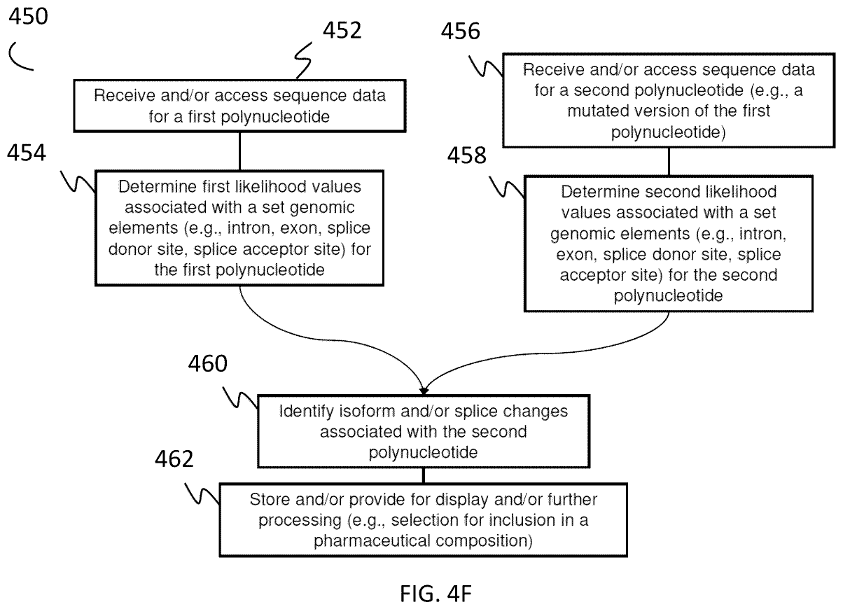

Turning to FIG. 4A, in certain embodiments, machine learning model 204 comprises one or more LMs 402. In certain embodiments, an LM is or comprises one or more recurrent models, such as long short-term memories (LSTMs), implemented alone or in combination, e.g., as in a bi-directional LSTM (bi-LSTM). In certain embodiments, an LM comprises one or more transformer models. Examples of LMs include, without limitation, evolutionary scale models (ESM), bidirectional encoder representations from transformers (BERT), and the like. In certain embodiments, LMs may comprise one or more members selected from the group consisting of an autoregressive LM, autoencoding LM, encoder-decoder LM, bidirectional LM, fine-tuned LMs, and multimodal LMs.

In certain embodiments, LMs are used to generate predictions about textual data representing, for example, a language. Languages processed by LMs include, without limitation, natural languages such as English, French, Thai, and the like. Of particular relevance to technologies of the present disclosure, LMs may also operate on biological sequences, treating them as languages. LMs may, accordingly, in certain embodiments, be used to analyze and generate predictions about biological sequences, such as nucleotide sequences, polypeptide sequences, and the like.

For example, LMs may be used to evaluate protein sequences, treating proteins as sentences and amino acids as words. In certain embodiments, machine learning models of the present disclosure utilize an LM approach in the context of nucleotide sequences, treating sequences of nucleotides as sentences and tokens representing k-mers (e.g., sets of k consecutive nucleotides) as words—a “genomic” LM.

In certain embodiments, for example, as illustrated in FIG. 4B, a genomic LM may be trained to receive, as input, nucleotide sequence data in which a nucleotide sequence is represented via a sequence of tokens 412a and predict values of unseen or masked tokens.

In certain embodiments, during training, a LM may be presented with example sequences 412b in which a fraction of the tokens are masked or with example sequences that are incomplete. For example, a central token of a sequence may be masked. For example, randomly 15% (e.g., 5%, 10%, 20%) of tokens within a sequence may be masked. A LM may then be tasked with predicting values of the masked tokens or next tokens in incomplete sequences. In this way, LMs can be trained in an unsupervised fashion, on unlabeled nucleotide sequence data.

For example, as illustrated in FIG. 4B, a LM may comprise one or more (e.g., a plurality of) transformer layers 414a, 414b, 414c and an output head that outputs a set of likelihood values 420 representing predicted likelihoods of various possible types (or values) of masked token 413. By comparing this output with ground truth values, available in the original sequence, a LM can be trained to generate accurate predictions.

In certain embodiments, LMs generate, for example internally, high-dimensional representations of biological sequences, referred to as embeddings. For example, for a given sequence received as input, an embedding may comprise, for each nucleotide or token of the given sequence, a vector having a plurality of values (e.g., numerical values). That is, given a nucleotide sequence, provided as a sequence of tokens to a genomic LM, the genomic LM may generate, for each token a corresponding N-dimensional embedding vector, N is an integer, corresponding to the dimension of the embedding.

As shown in FIG. 4B, these embeddings may be extracted, and used in and of themselves as input to one or more downstream models, for example as described in further detail herein. In certain embodiments, a set of embedding vectors is extracted from a particular layer of LM. In certain embodiments, an embedding 424c is extracted from a final transformer layer 414c. In certain embodiments, a set of embedding vectors is extracted from earlier transformer layers, as illustrated in FIG. 4B. In certain embodiments, multiple sets of embedding vectors are extracted and used as embeddings, e.g., each set of embedding vectors from a particular transformer layer.

In certain embodiments, a set of embedding vectors is extracted from one or more particular layers of a LM. For example, in certain embodiments, a LM may comprise a plurality of layers, and a set of embedding vectors may be extracted from a portion (e.g., not necessarily all) of the layers. For example, a single embedding vector may be extracted from a single layer, such as a final layer. In certain embodiments, a set of embedding vectors may be extracted from a plurality of layers [e.g., each embedding vector of the set corresponding to and extracted from a (e.g., different) particular layer]. In certain embodiments, a particular set of embedding layers and/or a particular set of LM layers from which embedding vectors are extracted may be determined via a probing approach. Probing may be used to assess a quality (e.g., performance) of a set of embedding vectors to solve one or more downstream tasks. For example, a LM may be trained, e.g., initially (e.g., pre-trained; e.g., to serve as a foundational model) on a first particular task, such as masked language modelling. In certain embodiments, one or more (e.g., each) layer of the LM may be probed to evaluate performance on several particular downstream tasks to evaluate the representation capabilities of the LM. For example, given a dataset of nucleotide sequences for a downstream task, such as promoter detection and enhancer detection, embedding vectors returned by one or more (e.g., five, ten, twenty) layers of LM may be computed and stored. The embedding vectors of each individual layer of LM may be used as inputs for several downstream models, such as logistic regression model and multi-layer perceptron, to solve the downstream task. A search over hyperparameters may be used for training the resulting models. For example, a model may be trained and validated using various hyperparameters, such as a learning rate, an activation function and a number of layers, to find a best performing model for a given layer of LM. Such hyperparameter search may be used to determine a best performing model associated with a specific layer of LM. The best performing models for various layers of LM may be further evaluated on a testing set. Model performances may be related with roles of associated LM layers on downstream task performance of the LM model. The obtained insights may be used to further revise and optimize LM and its architecture to enhance its performance on downstream tasks.

In certain embodiments, for example since sequence lengths can vary, a set of embedding vectors may be summed, averaged, or otherwise aggregated across sequence positions, for example, as described in in PCT publications WO 2022/235847 and WO 2022/235853, the content of which is hereby incorporated by reference in its entirety.

As described, for example in H. Dalla-Torre et al. 2023, the content of which is incorporated by reference herein in its entirety, embeddings generated by LMs trained on large amounts of genetic data encode valuable information about nucleotide sequences. Since genomic LMs can be trained in an unsupervised (or self-supervised) fashion via masked token or next token prediction approaches, they are able to leverage vast amounts of training data, which may include various nucleotide sequences available via public and/or proprietary sources such as the Human Genome Project (e.g., https://www.ncbi.nlm.nih.gov/datasets/genome/GCF_000001405.26/), the 1000 Genomes project, as well as various other datasets. As described in further detail herein, and demonstrated in Example 1 below, in certain embodiments, embedding vectors generated via a genomic LM can be leveraged and used as input to downstream models, such as a segmentation head, to offer improved performance on particular tasks.

C.ii. Combining LM Encoders with Segmentation Model(s)

In particular, turning again to FIG. 4A, in certain embodiments, a machine learning model of the present disclosure comprises one or more language model-based encoders and one or more segmentation heads. The one or more language model-based encoders may generate one or more embeddings based on nucleotide sequence data (e.g., and/or a tokenized version of the nucleotide sequence data) received as input. The one or more segmentation heads may determine a plurality of likelihood values based on the one or more embedding representations received as input.

In certain embodiments, a language model-based encoder comprises a transformer model. The encoder may transform a nucleotide sequence data (e.g., and/or a tokenized version of the nucleotide sequence data) into a sequence of embeddings. The encoder may add positional encodings to the sequence of embeddings. The sequence of embeddings may be processed by a at least a part of a transformer neural network (e.g., using one or more normalization and self-attention layers of a neural network). The encoder may be trained on any combination of DNA sequence data, RNA sequence data, and amino acid sequence data.

In certain embodiments, an encoder may be pre-trained. An encoder may be pre-trained in an unsupervised fashion, for example, using a training dataset comprising a plurality of example biological sequences [e.g., with the encoder having been trained to predict most likely tokens/nucleotides at masked positions in the plurality of example nucleotide sequences (e.g., masked language modeling (MLM))]. As described herein and illustrated in FIG. 4B, a pre-trained LM encoder may be used to generate embedding vectors, for use as input to segmentation head.

In certain embodiments, a segmentation head comprises a convolutional neural network (CNN) [e.g., a U-net architecture (e.g., a one-dimensional U-net architecture)]. A segmentation head may comprise a logistic regression model. A segmentation head may comprise a multi-layer perceptron (e.g., composed of up to two hidden layers). A segmentation head may be pre-trained. A segmentation head may output values associated with annotating a plurality of genomic elements in a nucleotide sequence. Each of the one or more segmentation heads may output values associated with annotating a single (e.g., different from other heads) genomic element in a nucleotide sequence data so that one or more segmentation heads output values associated with annotating multiple genomic elements in the nucleotide sequence.

In certain embodiments, machine learning models of the present disclosure utilize one or more segmentation heads. In certain embodiments, for example as illustrated in FIG. 4A, a single segmentation head generates, as output, a plurality of channels of likelihood values, each corresponding to a different genomic element and representing likelihoods that nucleotides belong to that genomic element. In certain embodiments, multiple segmentations heads may be used generating one or more output channels. In certain embodiments, each of at least a portion of the multiple segmentation heads generates a plurality of output channels. Among other things, in certain embodiments, such as ‘multi-task’ approach—i.e., whereby individual segmentation heads perform multiple labeling tasks can improve accuracy by taking advantage of transfer learning. In this way, such a multi-task approach benefits from model's shared knowledge across multiple tasks that can lead to improved performance. In contrast, segmentation approaches whereby a dedicated task specific model performs a single task to localize a single genomic element do not take advantage of transfer learning in this manner. Additionally, approaches taking advantage of the modular framework that facilitates multi-task learning described herein may readily be adapted to other tasks of localizing new genomic elements.

In certain embodiments, a machine learning model is (e.g., at least partially) trained while keeping some weights constant (e.g., frozen) (e.g., weights associated with one or more segmentation heads, weights associated with one or more encoders). Such training may result in improved results (e.g., faster training) in scenarios when, for example, weights associated with a particular part of a machine learning model are predominantly changing during the training as compared to other weights in the machine learning model.

In certain embodiments, at least a portion of a machine learning model is fine-tuned (e.g., using IA3 technique, e.g., from H. Liu et al. “Few-shot parameter-efficient fine-tuning is better and cheaper than in-context learning,” 2022). Specifically, at least a part of a pre-trained machine learning model (e.g., an encoder, a segmentation head) may be modified and further trained (e.g., in a supervised fashion) (e.g., separately from the remaining part of the machine learning model). For example, a segmentation head may be replaced by a classification or regression head. The segmentation or regression head may be further trained, for example, separately from the rest of the machine learning model. For example, weights of encoder layers may be kept constant (e.g., frozen) during training of a segmentation or regression head. For example, weights of encoder layers may be kept constant (e.g., frozen) and new, learnable weights are introduced. For example, for each transformer layer, three vectors with learnable weights may be introduced. The resulting model may be further trained for downstream tasks. As transformer weights are kept frozen, new introduced weights may “fine-tune” a model to a given task, achieving greater predictive ability. Overall, fine-tuning a part of a machine learning model may lead to performance improvements.

In certain embodiments, a machine learning model—for example, a genomic LM and/or a segmentation head, individually or in combination, as described herein, may be trained on a dataset comprising nucleotide sequence data from single species (e.g., human). In certain embodiments, a machine learning model is trained on a dataset including genetic variants arising from different human populations (e.g., Japanese, Tamil, British). For example, a dataset may include genetic variants occurring at a frequency of at least 1% in the human population. For example, a dataset may be associated with data from 1000 Genomes project (M. Byrska-Bishop et al. “High-coverage whole-genome sequencing of the expanded 1000 genomes project cohort including 602 trios” 2022). Such diversity of the dataset helps to capture representation of human genetic variation and may lead to better associated performance. A dataset may contain labels for individuals and human populations. For example, such labels may be useful when forming a training and/or validation set to ensure, for example, uniform sampling.

In certain embodiments, a machine learning model may be trained on a dataset comprising nucleotide sequence data from a plurality of species (e.g., two species, five species) [e.g., mouse (mm10), chicken (galGal6), fly (dm6), zebrafish (danRer11) and worm (ce11)].

The nucleotide sequence data may belong to a species that is not one of the one or more training species (e.g., the machine learning model performs zero-shot species inference) [e.g., gorilla (gorGor4), macaque (Mnem1), rat (mRatBN7), beaver (can genome v1), chinchilla (ChiLan1), whale (ASM228892v3), cat (Felis_catus_9), canary (SCA1), tetradon (TETRAODON8), anemonefish (AmpOce1), trout (fSalTru1) and Ciona intestinalis (KH)]. A training impact of each of the plurality of genomic species data may be weighted according to a genome size of each of the one or more training species (e.g., 5 for human, 4 for mouse, 2 for chicken, fly and zebrafish, and 1 for worm). In certain embodiments, a machine learning model may benefit from shared knowledge across species, which may boost performance, even on nucleotide sequences of individual species. Additionally or alternatively, training on nucleotide sequences belonging to multiple species may allow for improved performance if/when presented with nucleotide sequences belonging to unseen species at inference (e.g., zero-shot prediction).

Accordingly, as shown in FIG. 4C, in an example process 470, a sequence of tokens is obtained 472, corresponding to a sequence data representing, for example, one or more nucleotides. The sequence of tokens is processed 474 using an encoder portion of a machine learning model to generate a sequence of embeddings. Each embedding may correspond to or more nucleotides in the sequence data. The sequence of embeddings is processed 476 using a segmentation head of the machine learning model to determine, for example, for each nucleotide in the sequence data, a respective set of likelihood probabilities (e.g., values). The set of values may indicate whether a particular nucleotide is predicted to be associated with a respective genomic element.

C.iii. Sequence Size and Context

In certain embodiments, the nucleotide sequence data has a length of at least 100 kilobases (kb) (e.g., at least 50 kb, at least 30 kb, at least 20 kb, at least 10 kb, at least 6 kb, at least 3 kb). The length of the nucleotide sequence data may be determined by a context length. The context length is related to the ability of a machine learning model to handle sequences of similar length, for example, without a loss in performance as compared to sequences of smaller length. Longer context lengths leads to larger sequence context and, therefore, may result in improved performance. If a length of the nucleotide sequence data is larger than a context length of a machine learning model, the nucleotide sequence data may be divided into two or more partitions, each corresponding to a (e.g., distinct, non-overlapping) sub-sequence of the nucleotide sequence data. The machine learning model may process each sub-sequence separately/independently.

In certain embodiments, a machine learning model comprises a context length extension method (e.g., and rotary positional embeddings) (e.g., to rescale a frequency used in rotary positional embeddings, to overcome a limit of processing 12 kb sequence length imposed by a use of the rotary positional embeddings).

In certain embodiments, a maximum length of a training nucleotide sequence data used for training of a machine learning model has a length of at least 100 kilobases (kb) (e.g., at least 50 kb, at least 30 kb, at least 20 kb, at least 10 kb, at least 6 kb, at least 3 kb). The length of the nucleotide sequence data may be longer than the maximum length of the training nucleotide sequence data, thereby the machine learning model performing a zero-shot context extension.

C.iv. Encoder Architectures and Training Strategies

In certain embodiments, nucleotide sequence segmentation technologies of the present disclosure may utilize one or more of a variety of types of machine learning models and/or portions thereof as encoders 402 for generating embeddings to be provided to a segmentation head 404 as input, for example in the context of an architecture such as the one shown in FIG. 4A. As described herein, in certain embodiments encoder 402 is or comprises a LM-based encoder, that receives, as input, a sequence of tokens and generates, e.g., as an internal representation that is extracted, one or more embeddings—e.g., an embedding vector for each token of the input sequence. In certain embodiments, other encoder types may be used, additionally or alternatively.

In particular, as described and demonstrated, for example, in Example 2, encoder 402 may be or comprise convolutional layers. For example, turning FIG. 4D, in certain embodiments, an encoder may comprise a (e.g., first) set of convolutional layers 484a, 484b, 484c, etc., for example forming a first convolutional block or tower 484. First convolutional block or tower 484 may take an input sequence data 482 and progressively down-sample it to produce a first intermediate representation 486. In certain embodiments, first set of convolutional layers 484a, 484b, 484c etc. (collectively 484) is followed by a transformer block 488, comprising one or more transformer layers. Transformer block operates on first intermediate representation 486 and produces a second intermediate representation 490. In certain embodiments, a (e.g., second) set of convolutional layers 492a, 492b, 492c, etc. (e.g., a second convolutional block or tower 492) follows transformer block 488, taking second intermediate representation 490 and up-sampling it, to provide a final, higher resolution internal representation 494.

In certain embodiments, final, higher resolution representation may be used as an embedding representation and provided as input to segmentation head 404. In certain embodiments, other intermediate representations, such as output of any one of convolutional layers 492a, 492b, 492c, etc. following transformer block 488 and/or, additionally or alternatively, output, such as second intermediate representation 490, immediately following transformer block 488. Accordingly, as with language model 410 shown in FIG. 4A, a machine learning model 480 based on a combination of convolutional and self-attention (e.g., transformer) layers, as shown in FIG. 4D, may be used as an encoder in connection with the genomic element segmentation technologies of the present disclosure.

In certain embodiments, convolutional neural network-based encoder 480 may be trained by virtue of a final output layer 496 that transforms representation 494 into a desired output form, such as, for example, genomic tracks such as transcription factor (TF) chromatin immunoprecipitation and sequence (ChIP-seq), histone modification ChIP-sq, DNase-seq, ATAC-seq, cap analysis of gene expression (CAGE) track data, and the like. In certain embodiments, convolutional neural network-based encoder 480 may be trained in a supervised fashion, for example using a training dataset comprising a plurality of example sequences (e.g., DNA sequences) and target output values. Convolutional neural network-based encoder 480 may be, e.g., repeatedly tasked with receiving, as input, an example sequence or portion thereof and generating, as output 498, predicted output values. Based on a comparison between predicted output values and target output values, e.g., determined via a loss function, values of adjustable parameters withing various convolutional layers and transformer block 488 are adjusted, e.g., in an iterative fashion. Example optimization techniques include, e.g., a gradient-decent-based optimization technique, such as Adam described, for example, in Kingma and Ba, “Adam: A Method for Stochastic Optimization, arXiv:1312.6980, 2014 and Reynolds et al, “Open sourcing Sonnet—a new library for constructing neural networks. https://deepmind.com/blog/open-sourcing-sonnet (2017).

In certain embodiments, once trained (e.g., in a supervised fashion), an encoder model such as combined convolutional and self-attention-based model 480, may be combined with segmentation head 404 to create a genomic element segmentation model 204, as described herein. Genomic element segmentation model 204 may then be trained, e.g., in a supervised fashion, on labeled genomic element data. As described above, for example in section C.ii, in certain embodiments weights of encoder 402 are held fixed, while those of segmentation head 404 are allowed to vary and/or, in certain embodiments, weight of encoder 402 may also be allowed to vary (e.g., allowing them to be fine-tuned).

Example implementations of architectures as shown in FIG. 4D are described in Avsec et al., “Effective gene expression prediction from sequence by integrating long-range interactions,” Nature methods, vol. 18, no. 10, pp. 1196-1203, 2021 (hereinafter “Avsec, Nat. Meth., 2021”) and Linder et al., “Predicting RNA-seq coverage from DNA sequence as a unifying model of gene regulation,” bioRxiv, pp. 2023-08, 2023 (hereinafter “Linder, bioRxiv, 2023”), respectively.

In both, Avsec, Nat. Meth., 2021 and Linder, bioRxiv, 2023, machine learning models were implemented as stand-alone models, for purposes of generating predicted genomic track data—(i) not as encoders and (ii) without the capability of performing the detailed, multi-element and single nucleotide resolution annotations described herein. Example 2 below describes how the approaches described herein can be used to adapt and fine tune portions of the Enformer and Borzoi models described in Avsec, Nat. Meth., 2021 and Linder, bioRxiv, 2023 to be used as encoders as described above with respect to FIG. 4D, to create embeddings for use as input to segmentation heads in order to perform accurate single-nucleotide resolution segmentation of 14 classes of genomic elements in a fashion not contemplated in Avsec, Nat. Meth., 2021 and Linder, bioRxiv, 2023.

In certain embodiments, a convolutional neural network-based encoder 480 may not use any transformers. For example, as described in Kelly, “Cross-species regulatory sequence activity prediction,” PLOS Comput. Biol. 16, e1008050 (2020) architectures for tasks similar to the genomic track prediction tasks described in Avsec, Nat. Meth., 2021 did not use transformers (instead using dilated convolutions). Without wishing to be bound to any particular theory, however, it is believed that the attention mechanisms of transformer layers offers performance improvements. See, e.g., Avsec, Nat. Meth., 2021.

C.v. Alternative Splicing Variant Prediction

In certain embodiments, nucleotide sequence segmentation technologies of the present disclosure may be used to predict alternative splicing events, for example resulting from one or more mutations. For example, technologies of the present disclosure may, as described herein, be used to generate predictions representing whether particular nucleotides in a polynucleotide sequence belong to one or more particular genomic elements. As described herein, these predictions may take the form of sets of likelihood values determined for each nucleotide or group of nucleotides (e.g., a k-mer) in a sequence, with each likelihood value of a set corresponding to a particular genomic element.

In certain embodiments, predictions of certain genomic elements may be used (e.g., individually or in combination, collectively) to determine whether alternative splicing events may occur and what their impact on producing different isoforms may be. For example, likelihood values associated with genomic elements such as exons, introns, and splice sites (e.g., splice donor sites, splice acceptor sites) may be used, individually, and/or in combination with each other, to predict whether particular portions of polynucleotide sequences may produce alternative splice variants (e.g., isoforms) and, additionally or alternatively, what forms those splice variants may take.

For example, turning to FIG. 4E, genomic element segmentation technologies of the present disclosure include processes, such as example process 440, in which sequence data is received and/or accessed 442 and used to determine likelihood values associated with one or more genomic elements 444, such as introns, exons, splice donor sites and splice acceptor sites. The sequence data may, for example, represent a sequence of nucleotides of a polynucleotide and/or portion(s) thereof. The determined likelihood values may then be used to determine various RNA and/or protein isoforms that may be produced from the polynucleotide or portion(s) thereof 446. For example, likelihood values that represent predicted probabilities of nucleotides of a sequence of being part of exons and/or introns may be used to identify exons and introns throughout a polynucleotide sequence or portion thereof and, accordingly, used to determine potential isoforms resulting from, e.g., processes such as exon skipping, intron retention, inclusion/exclusion of mutually exclusive exons, etc. Additionally or alternatively, likelihood values that represent predicted probabilities of nucleotides being splice sites, such as splice donor and/or splice acceptor sites may be used to determine potential isoforms resulting from, e.g., processes such as exon skipping, intron retention, alternative donor and/or acceptor sites, inclusion/exclusion of mutually exclusive exons, etc. Determined isoforms may be stored and/or provided for display and/or further processing 448, for example for further testing, inclusion in a pharmaceutical composition, such as a vaccine, etc.

Turning to FIG. 4F, in certain embodiments, genomic element segmentation technologies of the present disclosure may be used in connection with processes, such as example process 450, for evaluating impact of mutations. For example, in certain embodiments, sequence data, for example, representing a sequence of a first polynucleotide or portion(s) thereof, may be received and/or accessed 452. Genomic element segmentation technologies as described herein may be used to determine first likelihood values associated with one or more genomic elements 454, such as introns, exons, splice donor sites and splice acceptor sites. In certain embodiments, impact of mutations, such as substitutions, insertions, deletions, etc., in the first polynucleotide sequence may be evaluated by receiving and/or accessing sequence data identifying the mutations and/or corresponding to a second, mutated version of the first polypeptide 456. Second, mutated, polynucleotide sequence may, accordingly, be segmented via approaches described herein to determine second likelihood values 458, indicative of, e.g., predicted likelihoods of various portions of second polynucleotide sequence being exons, introns, splice donor sites, splice acceptor sites, etc. Isoforms and/or impact of mutations on presence of splice sites may be determine based on first and second likelihood values 460. For example, as described in further detail herein, in Examples 1 and 2, changes in intron, exon, splice site (acceptor and/or donor) likelihoods at one or more positions in first and/or second nucleotide sequences may be determined. For example, differences in likelihood values (e.g., between first and second likelihood values) at or over one or more portions, such as at portions of first nucleotide sequence (relative to corresponding portions of second nucleotide sequence) identified as associated with particular introns, exons, splice acceptor sites, splice donor sites, etc. may be determined. Various difference metrics quantifying impact of mutations on alternative splicing events, such as, but not limited to donor site and/or acceptor site changes (e.g., gain or loss), e.g., summed or integrated over particular regions of first nucleotide sequence may, accordingly, be determined in this manner.

In certain embodiments, impact of mutations on alternative splicing may, for example, be used to identify candidate mutations (and/or isoforms resulting therefrom) as, e.g., drivers of disease and/or targets for treatment. For example, in certain embodiments, mutations (e.g., somatic mutations) occurring in tumor cells in cancer can be identified as giving rise to neoantigens and, accordingly, targets for inclusion in individualized cancer treatments. For example, individualized therapies for cancer, may, for example, identify neoantigens and/or epitopes thereof and deliver them, e.g., as peptides and/or via poly-ribonucleotides encoding them, e.g., as individualized cancer vaccines. Example approaches for individualized cancer vaccines following this approach are described, for example, in U.S. Publication No. 2014/0178438, entitled “Individualized Vaccines for Cancer,” and published Jun. 26, 2014, U.S. Publication No. US2019/0189241, entitled “SELECTING NEOEPITOPES AS DISEASE-SPECIFIC TARGETS FOR THERAPY WITH ENHANCED EFFICACY,” and published Jun. 20, 2019, and U.S. Publication No. US-2020-0209251-A1, entitled “METHODS FOR PREDICTING THE USEFULNESS OF DISEASE SPECIFIC AMINO ACID MODIFICATIONS FOR IMMUNOTHERAPY,” the contents of each of which are hereby incorporated by reference in their entirety.

C.vi. Compositions

In certain embodiments, genomic element segmentation technologies of the present disclosure may be used to identify sequences and/or candidate mutations therein (e.g., via alternative splicing variant prediction approaches described herein) for inclusion in constructs, e.g., for treatment of individual subjects.

For example, in certain embodiments, the present disclosure provides constructs (e.g., vaccine constructs) that comprise or encode nucleotide and/or polypeptide sequences corresponding to target genomic elements, such as genes or portions thereof, identified based on technologies of the present disclosure. Target genomic elements may be, for example, antigens or portions thereof (e.g., particular epitopes). Target genomic elements may be viral antigens or epitopes, or may be associated with cancer, for example, corresponding to cancer associated genes that are over-expressed in particular cancers and/or individualized neoantigens and/or portions thereof (e.g., neoepitopes). For example, a construct may comprise a polynucleotide, such as DNA or RNA, at least a portion of which encodes a gene or portion thereof comprising one or more candidate mutations, e.g., identified via approaches described herein. In certain embodiments, constructs encode neoepitope targets. These constructs may be designed and/or produced as described, for example, in WO 2012/159754, WO2018/224405, the content of each of which is hereby incorporated by reference in its entirety. Among other things, a construct may comprise a portion with one or more sub-regions, each having a particular polynucleotide sequence corresponding to a target genomic element having been identified and/or selected via approaches described herein. Upon administration to a subject, construct may be transcribed and/or translated, in-vivo, to for polypeptides that may, for example, act as neoantigens to elicit an immune response.

In some embodiments, a construct includes (e.g., encodes) any number of target genomic elements such that the number of nucleotides encoding all target genomic elements is approximately equal to a particular target length and/or with in a particular target range, e.g., about 1,000 nucleotides in length, about 1,200 nucleotides in length, about 1,300 nucleotides in length, about 1,400 nucleotides in length, about 1,500 nucleotides in length, e.g., between 1,000 and 1,500 nucleotides in length, etc.

In some embodiments, a construct includes up to about 2, 4, 6, 8, 10, 12, 14, 16, 18, 20, 22, 24, 26, 28, 30, or 50 target genomic elements. In some embodiments, a construct includes about 10 target genomic element. In some embodiments, a construct includes at least one target genomic element from each of about 2, 4, 6, 8, 10, 12, 14, 16, 18, 20, 22, 24, 26, 28, or 30 different genes. In some embodiments, a construct additionally includes one or more additional amino acid sequences, such as a secretory signal, a trafficking signal, and/or a linker, as described in further detail in WO 2012/159754, and WO2018/224405, the content of each of which is incorporated by reference herein in its entirety.

Compositions of the present disclosure may, in certain embodiments, be or comprise polyribonucleotides that encode one or more constructs described herein. Polyribonucleotide compositions may include features such as a 5′ cap, 5′UTR linker sequence, 3′ UTR, poly(A) tail, etc., as described in WO 2012/159754, WO2018/224405.

Exemplary formats useful for RNA compositions (e.g., pharmaceutical compositions) may include, among other things, non-modified uridine containing mRNA (uRNA), nucleoside-modified mRNA (modRNA), and self-amplifying mRNA (saRNA).

An exemplary polyribonucleotide encoding a vaccine construct may comprise a plurality of target genomic elements, including a 5′ cap analogue, a 5′ UTR, a coding sequence for a secretory signal (SEC), coding sequences for 10 target genomic element, a coding sequence for a control target genomic element, a coding sequence for an MITD, a 3′ UTR, and a polyA tail.

### D. Software, Computer System, and Network Environment

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning algorithms, such as an artificial neural network (ANN), random forest, decision trees, support vector machines, and the like, in order to determine, for a given input, one or more output values. In certain embodiments, the input comprises alphanumeric data which can include numbers, words, phrases, or lengthier strings, for example. In certain embodiments, the one or more output values comprise values representing numeric values, words, phrases, or other alphanumeric strings. In certain embodiments, the one or more output values comprise an identification of one or more response strings (e.g., selected from a database).

In certain embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In certain embodiments, once a machine learning module is trained, e.g., to accomplish a specific task such as identifying certain response strings, values of determined parameters are fixed and the (e.g., unchanging, static) machine learning module is used to process new data (e.g., different from the training data; e.g., infer a result) and accomplish its trained task without further updates to its parameters (e.g., the machine learning module does not receive feedback and/or updates). In certain embodiments, machine learning modules may receive feedback, e.g., based on automated review of accuracy or human user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC), field programmable gate arrays (FPGAs)).

In certain embodiments, machine learning modules implementing machine learning techniques may be composed of individual nodes (e.g., units, neurons). A node may receive a set of inputs that may include at least a portion of a given input data for the machine learning module and/or at least one output of another node. A node may have at least one parameter to apply and/or a set of instructions to perform (e.g., mathematical functions to execute) over the set of inputs. In certain embodiments, node instructions may include a step to provide various relative importance to the set of inputs using various parameters, such as weights. The weights may be applied by performing scalar multiplication (e.g., or other mathematical function) between a set of inputs values and the parameters, resulting in a set of weighted inputs. In certain embodiments, a node may have a transfer function to combine the set of weighted inputs into one output value. A transfer function may be implemented by a summation of all the weighted inputs and the addition of an offset (e.g., bias) value. In certain embodiments, a node may have an activation function to introduce non-linearity into the output value. Nonlimiting examples of the activation function include Rectified Linear Activation (ReLu), logistic (e.g., sigmoid), hyperbolic tangent (tanh), and softmax. In certain embodiments, a node may have a capability of remembering previous states (e.g., recurrent nodes). Previous states may be applied to the input and output values using a set of learning parameters.

A layer is a building block in a deep learning architecture composed of nodes. A layer is a set of nodes that receives data input (e.g., weighted or non-weighted input), transforms it (e.g., by carrying out instructions, e.g., applying a set of functions e.g., linear and/or non-linear functions), and passes transformed values as output (e.g., to the next layer). In certain embodiments, the set of nodes in a particular layer may share the same parameters and instructions without interacting with each other. A machine learning module may be composed of at least one layer (e.g., ordered). Examples of types of layers include convolutional layers (e.g., layers with a kernel, a matrix of parameters that is slid across an input to be multiplied with multiple input values to reduce them to a single output value); fully connected (FC) layers (e.g. all nodes are connected to all outputs of the previous layer); recurrent layers, long/short term memory (LSTM) layers, gated recurrent unit (GRU) layers (e.g., nodes with the various abilities to memorize and apply their previous inputs and/or outputs); batch normalization (BN) layers (e.g., layers that normalize a set of outputs from another layer, allowing for more independent learning of individual layers); activation layer (e.g., layers with nodes that only contain an activation function); (un) pooling layers [e.g., layers that reduce (increase) dimensions of an input by summarizing (splitting) input values in defined patches).

In certain embodiments, the performance of a machine learning module may be characterized by its ability to produce an output data that reproduces an input data with specific accuracy. To achieve specific accuracy, a training process is performed to find optimal parameters, such as weights, for every node in every layer of the machine learning module. In certain embodiments, the training process of a machine learning module may involve using output data to calculate an objective function (e.g., cost function, loss function, error function) that needs to be optimized (e.g., minimized, maximized). For example, a machine learning objective function may be a combination of a loss function and regularization parameter. The loss function is related to how well the output is able to predict the input. The loss function may take various forms, like mean squared error, mean absolute error, binary cross-entropy, categorical cross-entropy, for example. The regularization term may be needed to prevent overfitting and improve generalization of the training process. Typical regularization techniques include L1 Regularization or Lasso Regression, L2 Regularization or Ridge Regression, and Dropout (e.g., dropping layer outputs at random during training process).

In certain embodiments, objective function optimization of a machine learning module may involve finding at least one (e.g., all) of the present global optima (e.g., as opposed to local optima). A typical algorithm for objective function optimization follows principles of mathematical optimization for a multi-variable function and relies on achieving specific accuracy of the process. Examples of objective function optimization algorithms include gradient descent, nonlinear conjugate gradient, random search, Levenberg-Marquardt algorithm, limited-memory Broyden-Fietcher-Goldfarb-Shanno algorithm, pattern search, basin hopping method, Krylov method, Adam method, genetic algorithm, particle swarm optimization, surrogate optimization, and simulated annealing.

In certain embodiments, available input data includes training data and validation data, e.g., where the validation data is separate and non-overlapping with the training data. Training data is used during the training process to optimize a model, whereas validation data is used to check the accuracy of the model while operating on previously unseen data. In certain embodiments, training data is divided into batches (e.g., portions) that is sequentially used (e.g., in random order) as sets of inputs to train a model. In certain embodiments, a model is trained multiple times (e.g., epochs) on the entire set of training data.

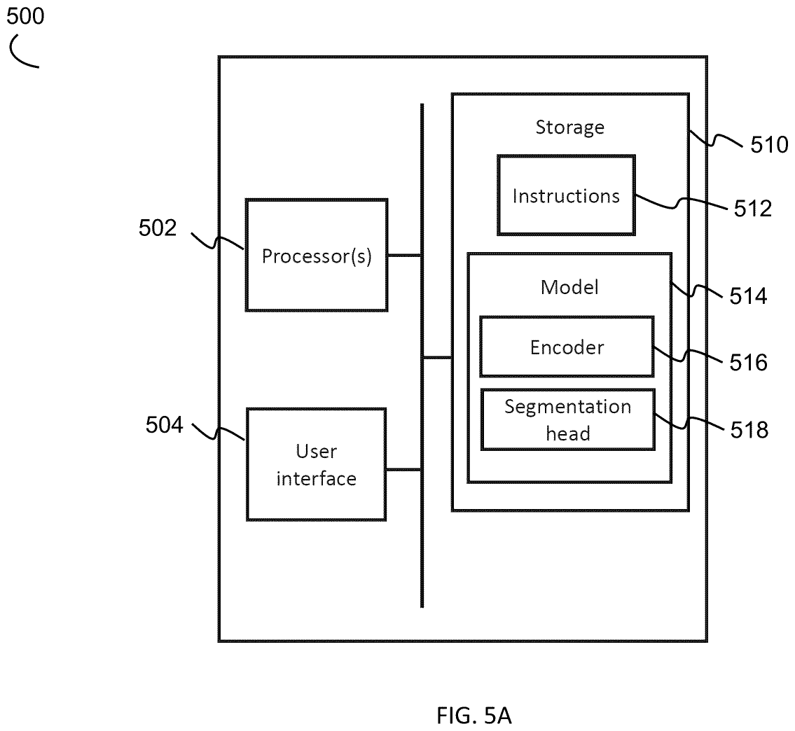

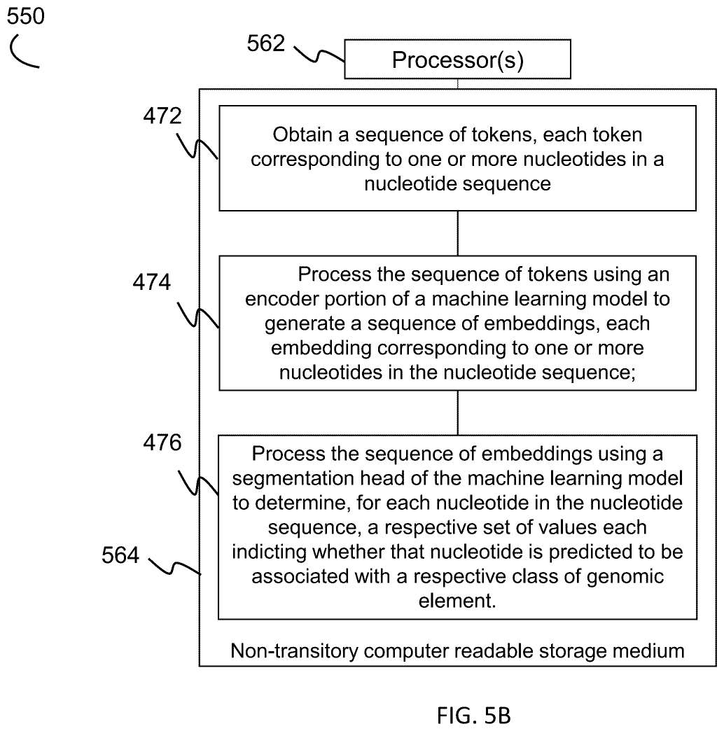

For example, turning to FIGS. 5A-5B, various processes and machine learning models used, e.g., in connection with processes described herein, may be included and/or stored in various computer systems and computer-readable media, in certain embodiments. FIG. 5A shows an exemplary system 500 for carrying certain methods of the present disclosure. The system 500 may comprise a processor 502, a user interface 504, and a storage medium 510 with stored instructions 512 as well as data (e.g., hyperparameters, weights) 514 associated with a machine learning model and its components, such as an encoder 516 and a segmentation head 518. FIG. 5B shows a system 550 for carrying methods of the present disclosure. The system may comprise a processor 562 and a storage medium 564. The storage medium 564 may store instructions related to methods of the present disclosure. For example, instructions associated with steps 472, 474, and 476 may be stored.

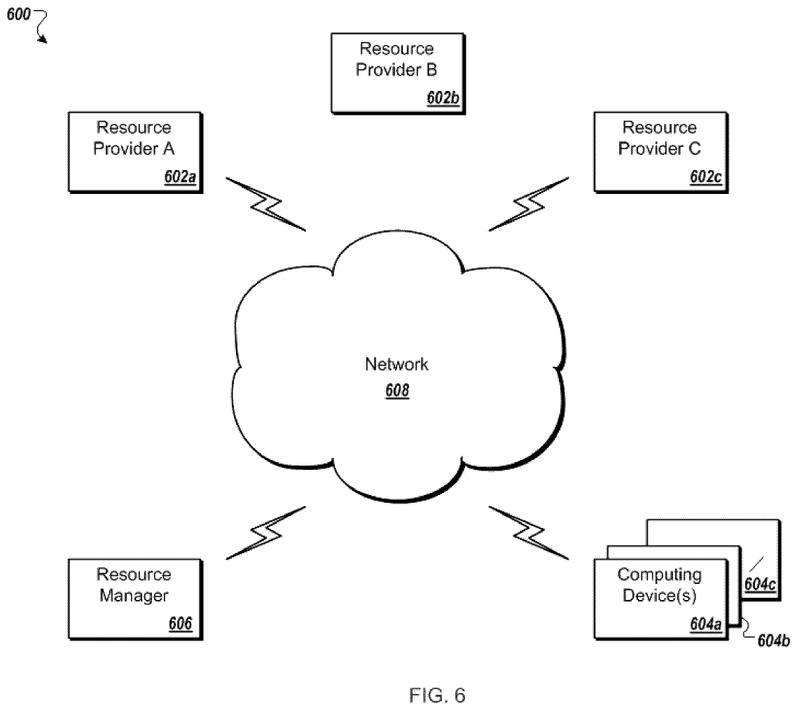

In certain embodiments, technologies of the present disclosure may be provided using a network environment. For example, as shown in FIG. 6, an implementation of a network environment 600 for use in providing systems, methods, and architectures as described herein is shown and described. In brief overview, referring now to FIG. 6, a block diagram of an exemplary cloud computing environment 600 is shown and described. The cloud computing environment 600 may include one or more resource providers 602a, 602b, 602c (collectively, 602). Each resource provider 602 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 602 may be connected to any other resource provider 602 in the cloud computing environment 600. In some implementations, the resource providers 602 may be connected over a computer network 608. Each resource provider 602 may be connected to one or more computing device 604a, 604b, 604c (collectively, 604), over the computer network 608.

The cloud computing environment 600 may include a resource manager 606. The resource manager 606 may be connected to the resource providers 602 and the computing devices 604 over the computer network 608. In some implementations, the resource manager 606 may facilitate the provision of computing resources by one or more resource providers 602 to one or more computing devices 604. The resource manager 606 may receive a request for a computing resource from a particular computing device 604. The resource manager 606 may identify one or more resource providers 602 capable of providing the computing resource requested by the computing device 604. The resource manager 606 may select a resource provider 602 to provide the computing resource. The resource manager 606 may facilitate a connection between the resource provider 602 and a particular computing device 604. In some implementations, the resource manager 606 may establish a connection between a particular resource provider 602 and a particular computing device 604. In some implementations, the resource manager 606 may redirect a particular computing device 604 to a particular resource provider 602 with the requested computing resource.

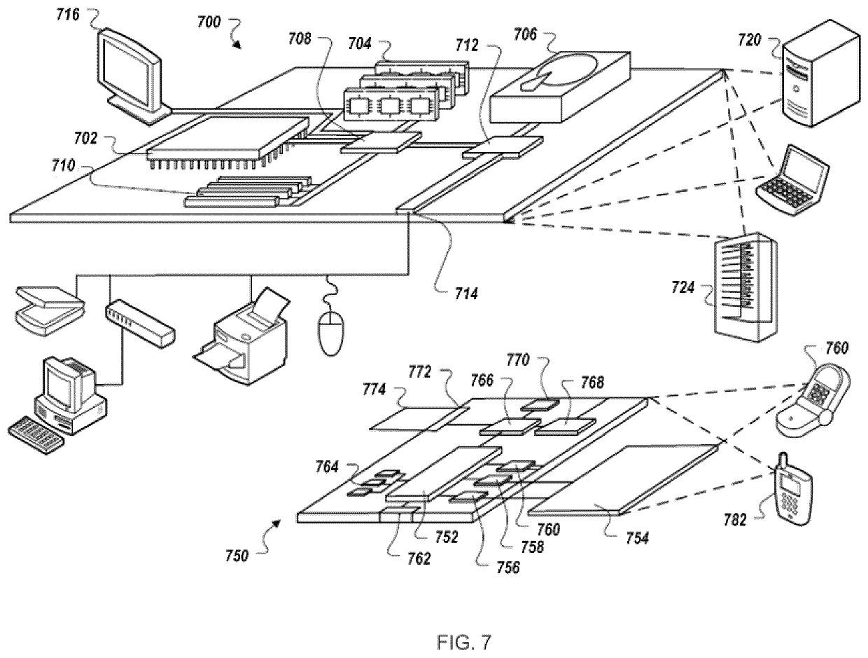

FIG. 7 shows an example of a computing device 700 and a mobile computing device 750 that can be used to implement the techniques described in this disclosure. The computing device 700 is intended to represent various forms of digital computers, such as laptops, desktops, workstations, personal digital assistants, servers, blade servers, mainframes, and other appropriate computers. The mobile computing device 750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 700 includes a processor 702, a memory 704, a storage device 706, a high-speed interface 708 connecting to the memory 704 and multiple high-speed expansion ports 710, and a low-speed interface 712 connecting to a low-speed expansion port 714 and the storage device 706. Each of the processor 702, the memory 704, the storage device 706, the high-speed interface 708, the high-speed expansion ports 710, and the low-speed interface 712, are interconnected using various busses, and may be mounted on a common motherboard or in other manners as appropriate. The processor 702 can process instructions for execution within the computing device 700, including instructions stored in the memory 704 or on the storage device 706 to display graphical information for a GUI on an external input/output device, such as a display 716 coupled to the high-speed interface 708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 704 stores information within the computing device 700. In some implementations, the memory 704 is a volatile memory unit or units. In some implementations, the memory 704 is a non-volatile memory unit or units. The memory 704 may also be another form of computer-readable medium, such as a magnetic or optical disk.

The storage device 706 is capable of providing mass storage for the computing device 700. In some implementations, the storage device 706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 702), perform one or more methods, such as those described above. The instructions can also be stored by one or more storage devices such as computer- or machine-readable mediums (for example, the memory 704, the storage device 706, or memory on the processor 702).

The high-speed interface 708 manages bandwidth-intensive operations for the computing device 700, while the low-speed interface 712 manages lower bandwidth-intensive operations. Such allocation of functions is an example only. In some implementations, the high-speed interface 708 is coupled to the memory 704, the display 716 (e.g., through a graphics processor or accelerator), and to the high-speed expansion ports 710, which may accept various expansion cards (not shown). In the implementation, the low-speed interface 712 is coupled to the storage device 706 and the low-speed expansion port 714. The low-speed expansion port 714, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 720, or multiple times in a group of such servers. In addition, it may be implemented in a personal computer such as a laptop computer 722. It may also be implemented as part of a rack server system 724. Alternatively, components from the computing device 700 may be combined with other components in a mobile device (not shown), such as a mobile computing device 750. Each of such devices may contain one or more of the computing device 700 and the mobile computing device 750, and an entire system may be made up of multiple computing devices communicating with each other.

The mobile computing device 750 includes a processor 752, a memory 764, an input/output device such as a display 754, a communication interface 766, and a transceiver 768, among other components. The mobile computing device 750 may also be provided with a storage device, such as a micro-drive or other device, to provide additional storage. Each of the processor 752, the memory 764, the display 754, the communication interface 766, and the transceiver 768, are interconnected using various buses, and several of the components may be mounted on a common motherboard or in other manners as appropriate.

The processor 752 can execute instructions within the mobile computing device 750, including instructions stored in the memory 764. The processor 752 may be implemented as a chipset of chips that include separate and multiple analog and digital processors. The processor 752 may provide, for example, for coordination of the other components of the mobile computing device 750, such as control of user interfaces, applications run by the mobile computing device 750, and wireless communication by the mobile computing device 750.

The processor 752 may communicate with a user through a control interface 758 and a display interface 756 coupled to the display 754. The display 754 may be, for example, a TFT (Thin-Film-Transistor Liquid Crystal Display) display or an OLED (Organic Light Emitting Diode) display, or other appropriate display technology. The display interface 756 may comprise appropriate circuitry for driving the display 754 to present graphical and other information to a user. The control interface 758 may receive commands from a user and convert them for submission to the processor 752. In addition, an external interface 762 may provide communication with the processor 752, so as to enable near area communication of the mobile computing device 750 with other devices. The external interface 762 may provide, for example, for wired communication in some implementations, or for wireless communication in other implementations, and multiple interfaces may also be used.

The memory 764 stores information within the mobile computing device 750. The memory 764 can be implemented as one or more of a computer-readable medium or media, a volatile memory unit or units, or a non-volatile memory unit or units. An expansion memory 774 may also be provided and connected to the mobile computing device 750 through an expansion interface 772, which may include, for example, a SIMM (Single In Line Memory Module) card interface. The expansion memory 774 may provide extra storage space for the mobile computing device 750, or may also store applications or other information for the mobile computing device 750. Specifically, the expansion memory 774 may include instructions to carry out or supplement the processes described above, and may include secure information also. Thus, for example, the expansion memory 774 may be provide as a security module for the mobile computing device 750, and may be programmed with instructions that permit secure use of the mobile computing device 750. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 752), perform one or more methods, such as those described above. The instructions can also be stored by one or more storage devices, such as one or more computer- or machine-readable mediums (for example, the memory 764, the expansion memory 774, or memory on the processor 752). In some implementations, the instructions can be received in a propagated signal, for example, over the transceiver 768 or the external interface 762.

The mobile computing device 750 may communicate wirelessly through the communication interface 766, which may include digital signal processing circuitry where necessary. The communication interface 766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 768 using a radio-frequency. In addition, short-range communication may occur, such as using a Bluetooth®, Wi-Fi™, or other such transceiver (not shown). In addition, a GPS (Global Positioning System) receiver module 770 may provide additional navigation- and location-related wireless data to the mobile computing device 750, which may be used as appropriate by applications running on the mobile computing device 750.

The mobile computing device 750 may also communicate audibly using an audio codec 760, which may receive spoken information from a user and convert it to usable digital information. The audio codec 760 may likewise generate audible sound for a user, such as through a speaker, e.g., in a handset of the mobile computing device 750. Such sound may include sound from voice telephone calls, may include recorded sound (e.g., voice messages, music files, etc.) and may also include sound generated by applications operating on the mobile computing device 750.

The mobile computing device 750 may be implemented in a number of different forms, as shown in the figure. For example, it may be implemented as a cellular telephone 780. It may also be implemented as part of a smart-phone 782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, various modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein. Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

### E. Example 1: Implementation and Performance Analysis of Exemplary DNA Segmentation Model—SegmentNT

This example describes and demonstrates performance of an exemplary machine learning system for annotating DNA sequences, denoted “SegmentNT,” in accordance with certain embodiments of systems and methods described herein.

The intersection of genomics research and deep learning methods is profoundly changing the ability to understand the information encoded in each of the 3 billion nucleotides in the human genome and to accurately assess their influence with respect to different gene-regulatory activity layers, ranging from regulatory elements and transcriptional activation to splicing and polyadenylation (G. Eraslan et al. 2019; T. Yue et al. 2023). Sequence-based machine learning models trained on large-scale genomics data capture complex patterns in the sequence and can predict diverse molecular phenotypes with great accuracy. Recently, convolutional neural networks have demonstrated superior performance over other architectures across most sequence-based problems (J. Zhou et al. 2015; B. Alipanahi et al. 2015; D. R. Kelley et al. 2016; D. R. Kelley et al. 2018; D. R. Kelley et al. 2020; Z. Avsec et al. 2021; B. P. de Almeida et al. 2022; J. Linder et al. 2022; V. Agrawal et al. 2022), sometimes combined with LSTMs (F Stiehler et al. 2020; M. R. Amin et al. 2018; D. Quang et al. 2016; L. Minnoye et al. 2020) or transformer layers (Z. Avsec et al. 2021; J. Linder et al. 2023).

Most genomics models are built with a focus on only one specific task where one task is to annotate that a gene segment belongs to a specific group of genomic elements, for example detecting the presence of promoter elements in a given input sequence (M. Oubounyt et al. 2019) or the binding of transcription factors (Z. Avsec et al. 2021). Given the diversity and complexity of the different gene-regulatory activity processes, models that can tackle different types of tasks simultaneously will be easier to adopt by the community and should also obtain higher performance on each task by leveraging shared knowledge between tasks. Models compatible with different types of tasks have emerged using either multitask supervised training schemes from scratch (D. R. Kelley et al. 2016; J. Zhou et al. 2015; K. M. Chen et al. 2022; Z. Avsec et al. 2021; J. Linder et al. 2023) or making use of large pre-trained DNA foundation models that are afterwards finetuned towards specific tasks (Y. Ji et al. 2021; Z. Zhou et al. 2023; H. Dalla-Torre, et al. 2023; E. Nguyen et al. 2023; G. Benegas et al. 2023; V. Fishman et al. 2023). This last approach in particular is very promising for genomics given the ability of such foundation models to be trained on unlabeled data (e.g., raw genomes or experimental sequencing data), creating general-purpose representations capable of solving a multitude of downstream tasks, similarly to what has been observed in other fields such as natural language processing and computer vision (J. Devlin et al. 2018; A. Radford et al. 2019; H. Bao et al. 2022; S. Gidaris et al. 2018; A. Radford et al. 2021).

A second limitation of most genomics models trained on certain tasks, such as detecting promoter elements in an input sequence, is their limited resolution, usually predicting a single probability or quantitative score for the whole candidate sequence (M. Oubounyt et al. 2019) or low-resolution continuous signals averaged across windows of 100-200 base pairs (Z. Avsec et al. 2021). While framing such tasks as classification has practical advantages, this formulation has its limits in practice where knowing precisely where elements are located in the sequence is valuable and/or required. In addition, such a model does not make use of additional information related to the spatial position of such elements. Models that make predictions at nucleotide-resolution were shown to improve performance and recover better features over previous deep learning classification approaches on tasks related to transcription factor binding (Z. Avsect et al. 2021), chromatin accessibility (A. E. Trevino et al. 2021; S. Nair et al. 2023) and RNA polyadenylation (J. Linder et al. 2022). Developing models that can solve multiple tasks and at this nucleotide-level resolution is thus a promising avenue for the field.

In this example a machine learning model is trained to predict the location of several types of genomics elements in a sequence at single-nucleotide resolution, in accordance with certain embodiments of the present disclosure. As demonstrated herein, this approach both improves the model detection performance and provides more refined annotations and predictions for an input sequence. Among other things, the approach described herein leverages the insight that localizing elements at nucleotide resolution in a DNA sequence can be viewed as analogous to localizing objects in images at pixel resolution, usually referred to as segmentation task (O. Ronneberger et al. 2015; T.-Y. Lin et al. 2017; J. Redmon et al. 2016). Based on this analogy, a segmentation architecture based on those used in image segmentation was adapted herein. More specifically, this example demonstrates a DNA segmentation model, the Segment-Nucleotide Transformer (SegmentNT), that combines the pre-trained DNA foundation model Nucleotide Transformer (NT) (H. Dalla-Torre et al. 2023) and a ID U-Net (O. Ronnerberger et al. 2015) architecture. SegmentNT was further trained to predict the location of 14 types of human regulatory and gene elements in input sequences up to 30 kb at single-nucleotide resolution. SegmentNT achieves high nucleotide accuracy for all elements and generalizes to input sequences up to 50 kb. The best SegmentNT-30 kb model was further finetuned on multiple species, showing improved generalization to unseen species.

No other model capable of predicting element locations at the nucleotide level for different sorts of elements, including gene and regulatory elements, has been developed so far, except for acceptor and donor splice sites identification (K. Jaganathan et al. 2019; T. Zheng and Y. I. Li 2022) or cross-species gene annotation (F. Stichler et al. 2020). Given the complexity of the above task, the SegmentNT model demonstrates the benefit of leveraging pre-trained DNA foundation models over specialized methods trained from raw DNA sequences, showcasing the power of foundation models to tackle complex tasks in genomics and at single-nucleotide resolution.

E.i. SegmentNT: Finetuning Nucleotide Transformer for Segmentation of DNA Sequences at Nucleotide Resolution

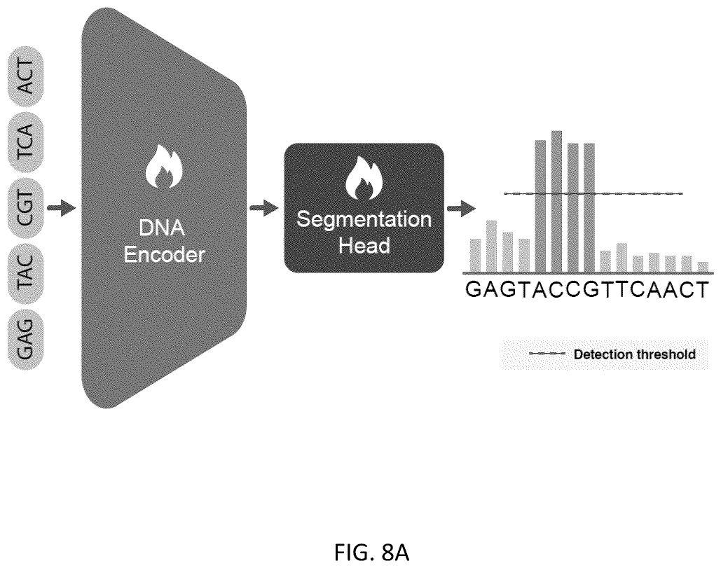

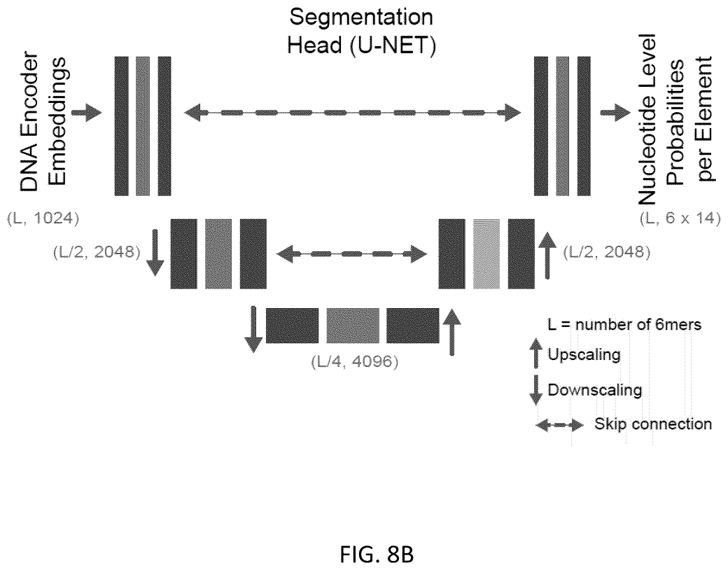

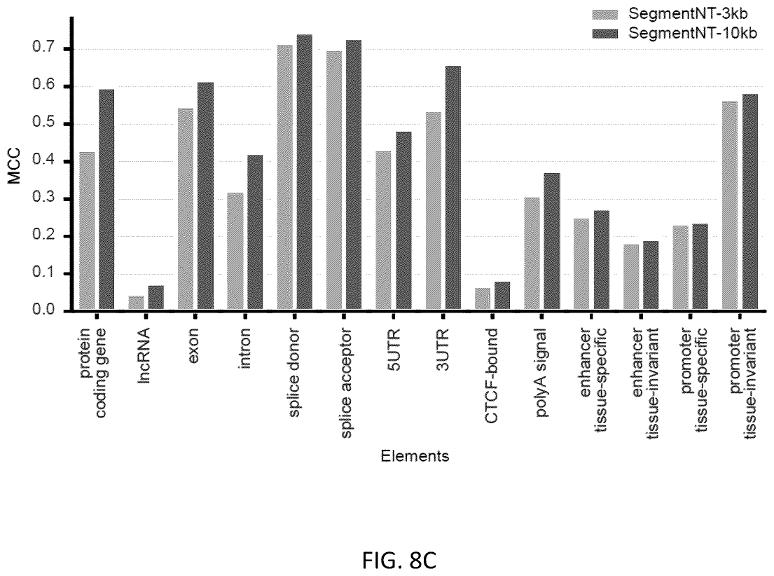

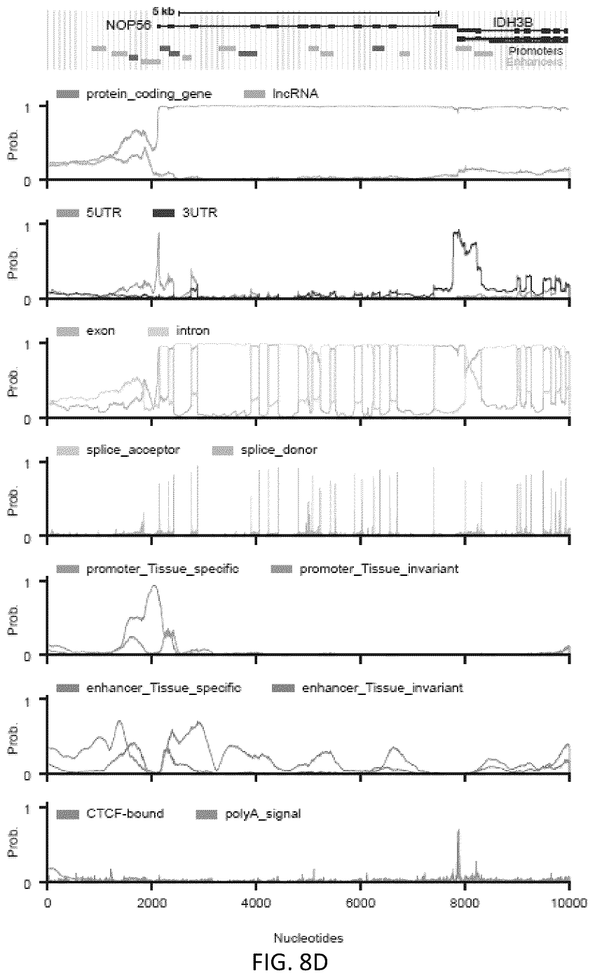

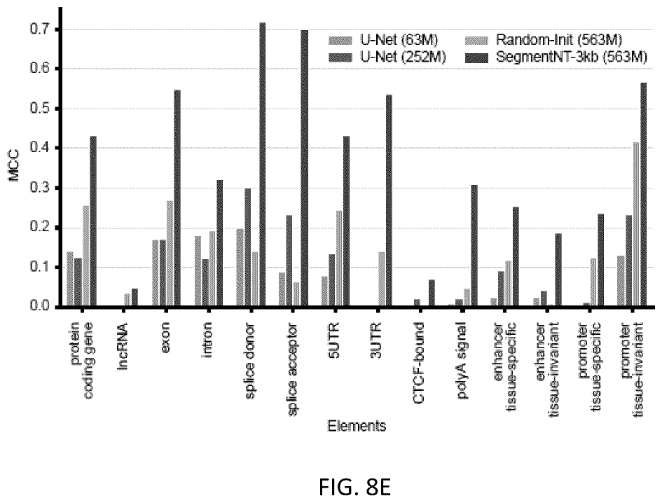

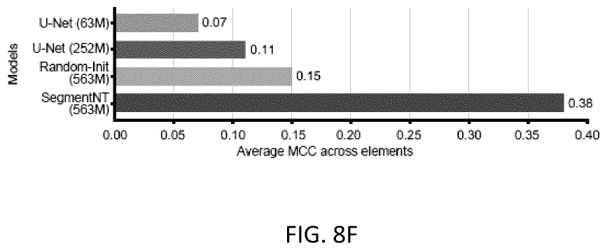

SegmentNT is a DNA segmentation model that combines the pre-trained DNA foundation model Nucleotide Transformer (NT) (H. Dalla-Torre et al. 2023) and a segmentation head to detect elements at different scales as shown in FIG. 8A. As segmentation head a ID U-Net architecture is used that downscales and upscales the foundation model embeddings of the input DNA sequence as shown in FIG. 8B (J. Linder et al. 2023). This architecture is trained end-to-end on a dataset of genomic annotations to minimize a focal loss objective (T.-Y. Lin et al. 2017) to deal with element scarcity in the dataset.

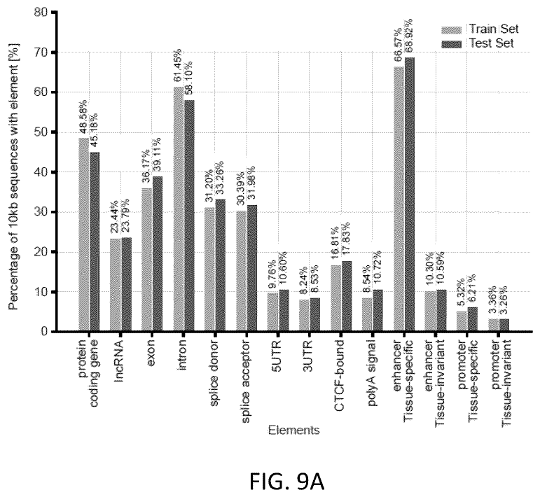

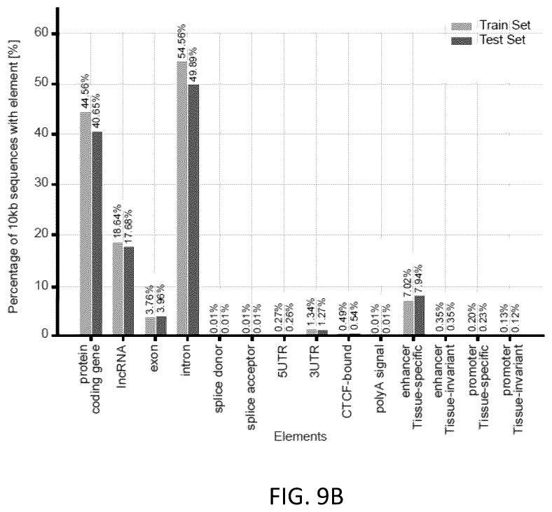

To train SegmentNT a dataset of annotations at nucleotide-level precision was curated for 14 types of genomic elements in the human genome derived from GENCODE (J. Harrow et al. 2012) and ENCODE (ENCODE Project Consortium, 2012), including gene elements (protein-coding genes, lncRNAs, 5′UTR, 3′UTR, exon, intron, splice acceptor and donor sites) and regulatory elements (polyA signal, tissue-invariant and tissue-specific promoters and enhancers, and CTCF-bound sites) as shown in FIGS. 9A-9B. Since these element annotations can overlap, SegmentNT predicts the probability of belonging to each of the genetic elements at nucleotide level. For example, in different gene transcript isoforms the same DNA region can be considered an exon or an intron, enhancers can also be found in gene regions, and polyA signals are usually in the genes 3′UTRs. In addition, here the canonical definition of exons is used as any part of a gene that can be present in the final mature RNA after introns have been removed by RNA splicing, thus also overlapping with 5′ and 3′UTRs. This allows the prediction of every genomics element independent of the other predictions. The annotation of all promoter and enhancer regions in the human genome was derived from the latest registry of candidate cis-regulatory elements by ENCODE (ENCODE Project Consortium, 2020). It contains 790 k enhancers and 34 k promoters grouped by their activity in different tissues.

A model was first trained to segment these distinct 14 genomics elements in input DNA sequences of 3 kb (SegmentNT-3 kb). This model was further finetuned on 10 kb input sequences (SegmentNT-10 kb) to extend its input length. This was achieved by initializing SegmentNT-10 kb from the best checkpoint of the SegmentNT-3 kb model for a more efficient training and length-adaptation. For a given input sequence, these models make 42,000 and 140,000 predictions, respectively, each being the probability of a given nucleotide to belong to a genomics element type. Model training, validation and performance evaluation were performed on different sets of chromosomes from the human genome to ensure no data leakage between the different sets in order for the test set to provide a robust evaluation of model performance. SegmentNT-3 kb demonstrated high accuracy in localizing these elements to nucleotide precision, showing a Matthews correlation coefficient (MCC) on the test set above 0.5 for exons, splice sites, 3′UTRs and tissue-invariant promoter regions. LncRNA and CTCF-binding sites were the most difficult elements to predict, with test MCC values below 0.1. Superior performance of the model was observed in sequences of 10 kb (average MCC of 0.43) compared with 3 kb (0.38), in particular for protein-coding genes, 3′UTRs, exons and introns, suggesting that these elements depend on longer sequence contexts as shown in FIG. 8C.

To further evaluate predictive performance, regions of the held-out test chromosomes were inspected. Evaluating SegmentNT-10 kb on a 10 kb window that covers the gene NOP56 on the positive strand and the end of the gene IDH3B on the negative strand shows that the model accurately predicts the different genic elements of each gene as shown FIG. 8D. SegmentNT correctly predicts both genes as protein-coding, their 5′UTR and 3′UTR positions, their splice sites and exon-intron structure, and also the polyA signals. In addition, SegmentNT captures the promoter region of NOP56, both the tissue-specific and tissue-invariant ones. This region also contains multiple enhancers and some of those are correctly predicted by the model. Still, although the global performance metric for enhancers is good (MCC of 0.27 for tissue-specific and 0.19 for tissue-invariant for SegmentNT-10 kb), enhancer predictions were more noisy. This could be related to their higher sequence complexity and diversity, and grouping them by cell type-specific activity should further improve model performance.

E.ii. Using Nucleotide Transformer as a Pre-Trained DNA Encoder Improves Training Efficiency Training and Performance

The model architecture and the importance of using the NT pre-trained foundation model as a DNA encoder was further evaluated. The performance of SegmentNT was compared with three different model architectures, using 3 kb input sequences for a simpler comparison. The NT DNA encoder was removed and two 1D U-Net architectures that take one-hot encoded DNA sequences directly as input instead of the NT embeddings were trained. One with the same 63M parameters of the head of SegmentNT and a larger model with an additional downsampling/upsampling block featuring a total of 252M parameters. For each model, the checkpoint was selected with the highest performance on the validation set and evaluated all on the same test set sequences. These two U-Net architectures demonstrated substantially reduced performance across all elements, with an average MCC of 0.07 (66M) and 0.11 (250M) compared with 0.38 for SegmentNT-3 kb, demonstrating the value of using a pre-trained DNA encoder as shown in FIGS. 8E-8F. The largest U-Net architecture used here (252M) is around half the parameter size of SegmentNT (563M) with a size comparable to the Enformer (J. Avsec et al. 2021).

To test the benefit of pretraining the NT foundation model, a model was trained with the same architecture as SegmentNT but using a randomly initialized NT DNA encoder model, rather than the pre-trained one. While for SegmentNT-3 kb model convergence was observed after 20M training sequences (10B tokens), the version with random initialized NT showed much slower convergence and have not converged yet even after 68M training sequences (34B tokens), a training more than three times longer. In addition, even after this longer training, performance of the randomly initialized model (average MCC 0.15) was substantially lower than SegmentNT-3 kb (0.38) across all 14 genomics elements as shown in FIGS. 8E-8F. In summary, SegmentNT demonstrates the value of DNA foundation models for solving challenging tasks in genomics such as localizing different types of genomics elements at a single nucleotide resolution.

E.iii. SegmentNT Outperforms Alternative Approaches in Predicting Regulatory Elements with Nucleotide-Precision

Next, the ability to predict regulatory elements as compared to alternative approaches was evaluated. To the best of inventors' knowledge, there are no models that can predict the location of regulatory elements in an input sequence at nucleotide resolution. Two approaches were considered that could be used to tackle this problem: sliding a binary classifier over the input 10 kb sequence and using the Enformer (J. Avsec et al. 2021) chromatin predictions as a surrogate for regulatory elements. For a more direct comparison with the SegmentNT model, the Nucleotide Transformer models (H. Dalla-Torre et al. 2023) finetuned on promoter or enhancer sequences were used as binary classifiers. These approaches were compared for the prediction of tissue invariant promoters and tissue-specific enhancers on 10 kb input sequences as these were the classes with the highest sequence predictive value as shown in FIG. 8C.

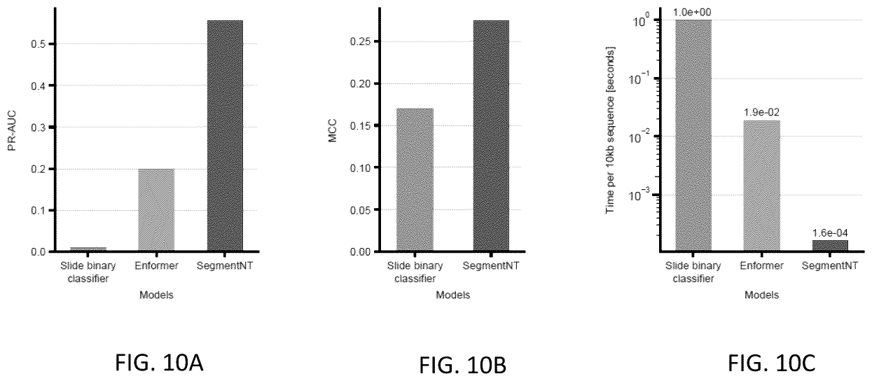

On predicting promoters at nucleotide precision, SegmentNT-10 kb outperformed both approaches as shown in FIGS. 10A-10B. Using the promoter finetuned model from NT (H. Dalla-Torre et al. 2023) and sliding it through each 10 kb sequence yielded very low performance, which can be related with the different set of promoter sequences used for training such model. To use the Enformer, the predictions of DNA accessibility were calculated for 7 different cell lines for each 10 kb sequence at its original 128 bp resolution bins and averaged to get a more robust DNA regulatory activity metric. Despite the different approach and dataset, this resulted in a good performance of 0.21 Precision-Recall Area Under the Curve (PR-AUC) for the prediction of promoter regions, but still well below SegmentNT-10 kb with a PR-AUC of 0.56 as shown in FIG. 10A. For predicting enhancers, the NT model finetuned on human enhancers (H. Dalla-Torre et al. 2023) was compared with the above sliding window approach. Here the performance was better than for promoters, with an MCC of 0.17, but again SegmentNT-10 kb achieved much better performance at 0.27 as shown in FIG. 10B.

In addition to providing advantages over previous approaches for predicting regulatory elements and achieving state-of-the art performance, SegmentNT is also much faster on inference. A SegmentNT-10 kb model segments the 14 genomics elements in an input 10 kb sequence (meaning 140,000 predictions) in 0.16 milliseconds. This inference time is about 100× faster than running the Enformer model (18 milliseconds) and 5000× faster than sliding a similar-size binary classifier model over the sequence (1 second) (all times using Jax code and in a single A100 GPU) as shown in FIG. 10C. For Enformer, the 10 kb sequences were padded for inference, since the original model predicts scores for 114,688 bp.

E.iv. SegmentNT Generalizes to Sequences Up to 50 kb

The ability to extend the sequence context length of SegmentNT was further investigated, motivated by the improved results observed for SegmentNT-10 kbp over SegmentNT-3 kbp as shown in FIG. 8C. However, NT uses rotary positional embeddings (RoPE; J. Su et al. 2021) which was set to support sequences up to 12 kb during its pre-training. As such, and given the periodic nature of ROPE encoding, using NT directly on sequences longer than 12 kb, whether for finetuning or inference, would yield poor performance. To address this problem, recent approaches were explored that have been proposed for extending contexts of ROPE models by converting the problem of length extrapolation into one of “interpolation”. Specifically, a context length extension method was employed that was first formally described in B. Peng et al. 2023, where the frequency used in RoPE embeddings is re-scaled to account for longer sequences (E. Trop et al. 2024; Y. Schiff et al. 2024). This approach can be used for extending the context length of SegmentNT during training to train it on sequences longer than 12 kb but also for performing inference with SegmentNT models on sequences longer than the ones seen during training.

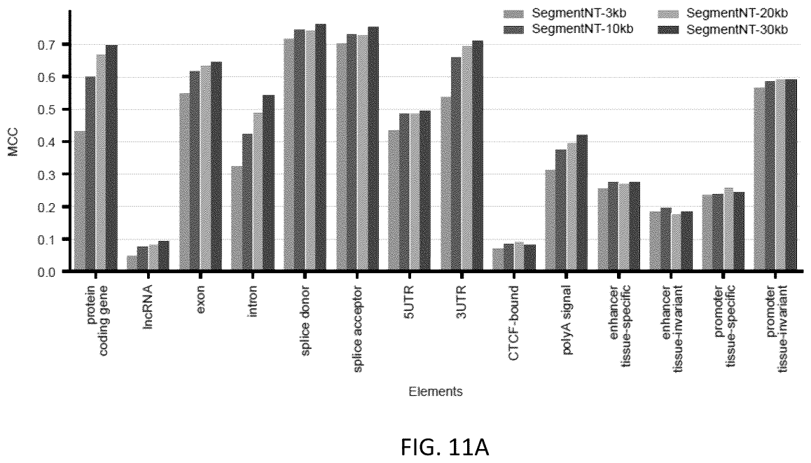

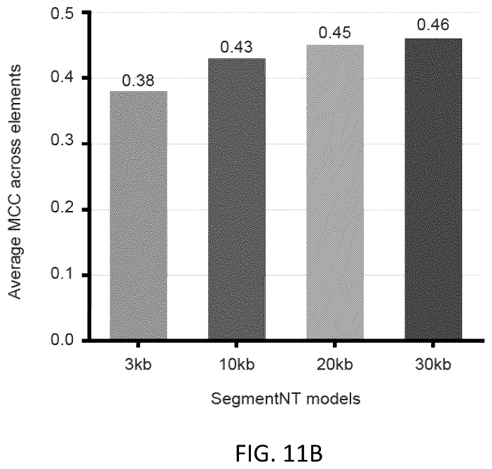

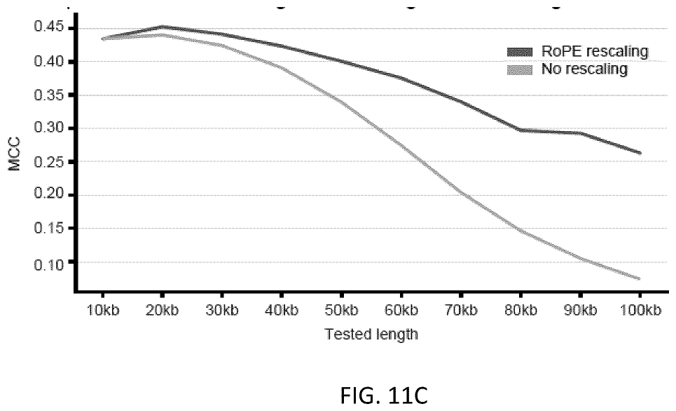

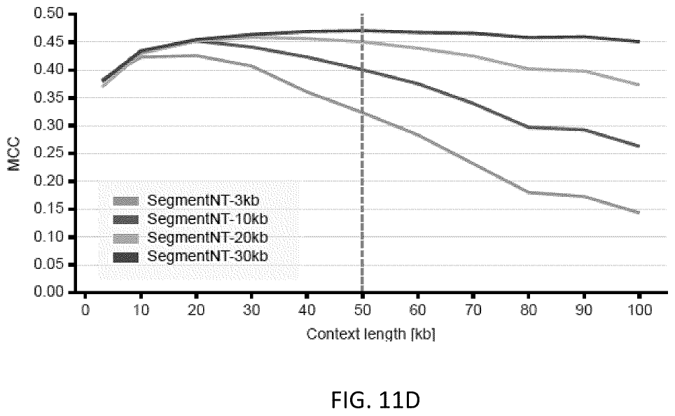

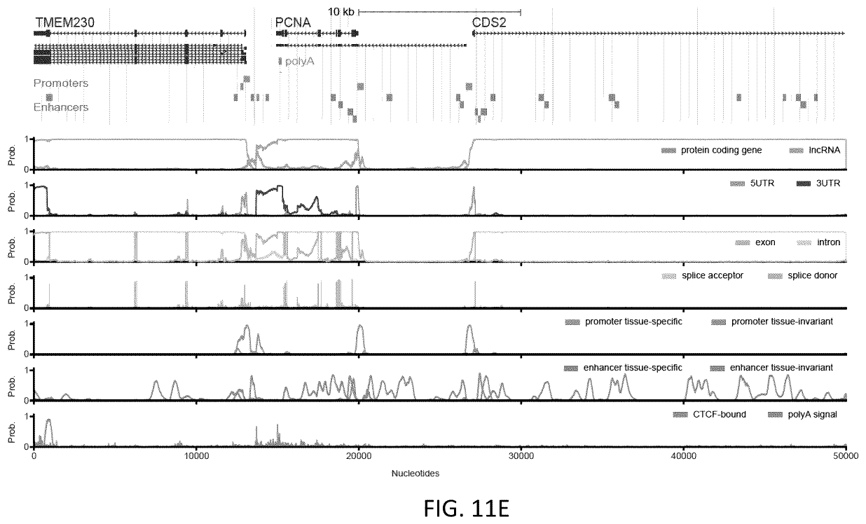

Context length extension in NT was implemented and two additional SegmentNT models were trained to segment the 14 genomics elements in DNA sequences of 20 kb (SegmentNT-20 kb) and 30 kb (SegmentNT-30 kb). Evaluation on the same test chromosomes showed consistent improvements in performance with increased sequence length, in particular for the segmentation of protein-coding genes, 3′UTRs, exons and introns as show in FIG. 11A. The model with the best performance across all elements was SegmentNT-30 kb with an average MCC of 0.46 as shown in FIG. 11B.

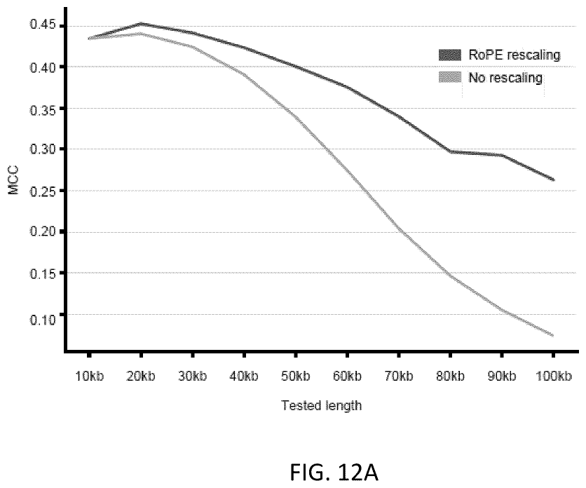

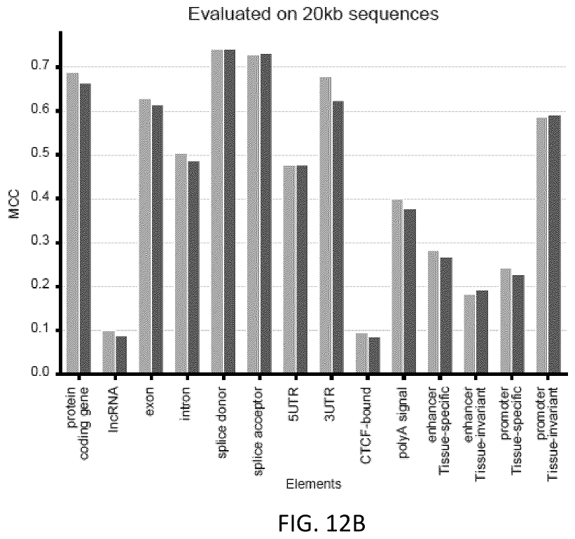

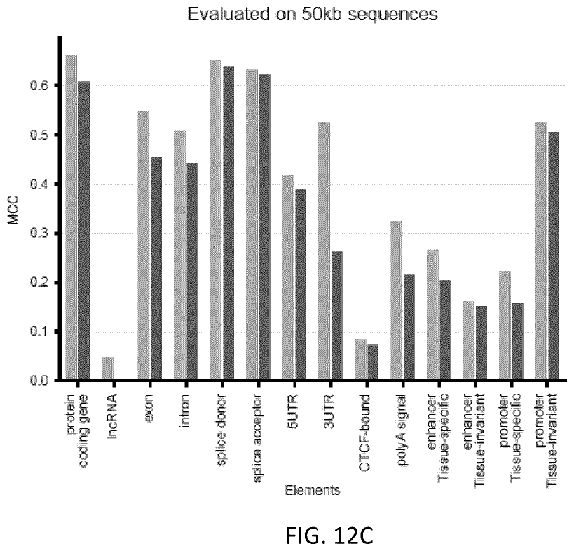

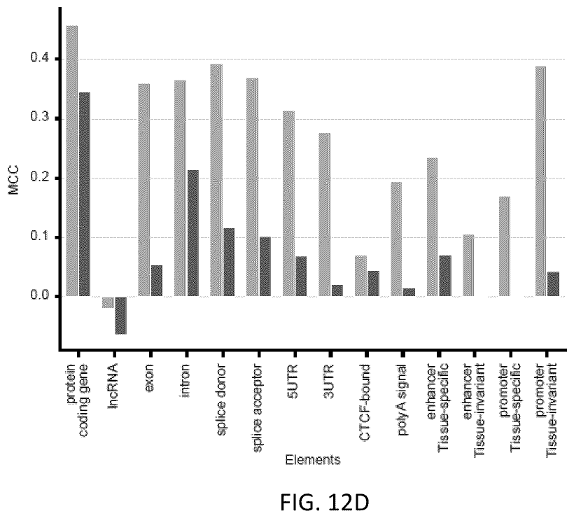

Since it is computationally expensive to finetune SegmentNT on even longer sequence lengths, the ability to leverage context length extension to evaluate a model pre-trained on a given length on longer sequences was tested. This approach was tested on the SegmentNT-10 kb model by evaluating it with or without context length extension on the prediction of sequences up to 100 kb from the same test chromosomes as shown in FIGS. 11C and 12A-12D. Context length extension substantially improved the performance of the model on longer sequences, in particular on 100 kb where the original model showed very poor performance (average MCC of 0.26 vs 0.07, respectively).

An extent to which context of different SegmentNT models could be extended was also tested by evaluating performance of all trained SegmentNT models (3 kb, 10 kb, 20 kb and 30 kb) on input sequence lengths between 3 and 100 kb using context length extension interpolation when needed. When averaging the performance across 14 elements, this revealed that the model trained on the longest context length (SegmentNT-30 kb) achieved the best results when evaluated in all context lengths, including shorter sequences as shown in FIG. 11D. Top performance was observed for 50 kb input sequences (average MCC of 0.47) and a drop in performance for sequences longer than 50 kb, although SegmentNT-30 kb still has good performance on sequences of 100 kb (0.45) as shown in FIG. 11D. These results highlight the flexibility of SegmentNT and how it can be applied to sequences of different lengths. The SegmentNT-30 kb model when segmenting the 14 genomics elements in an 50 kb input sequence makes 700,000 predictions at once (14×50,000), thus providing a very rich segmentation output. Representative examples of the SegmentNT-30 kb predictions for a 50 kb locus in the test set with three overlapping genes are shown in FIG. 11E.

E.v. Segment-NT Accurately Predicts Splice Sites and Mutations

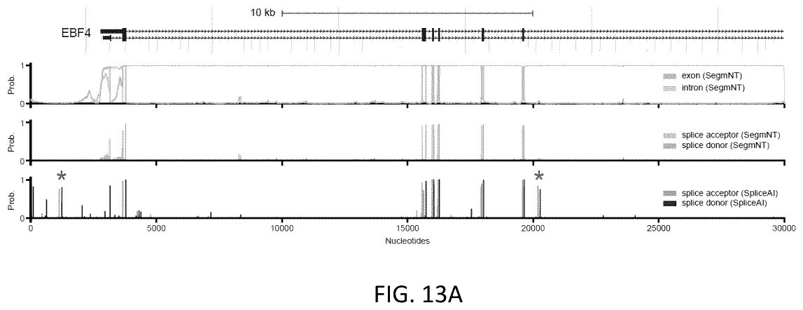

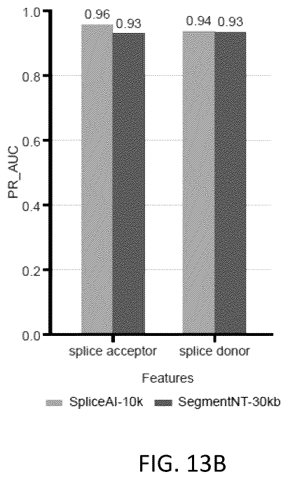

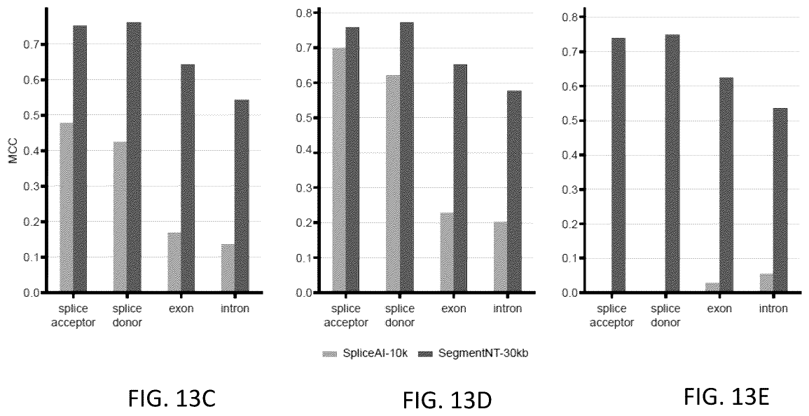

One of the main nucleotide-level tasks in genomics that has been tackled by previous models is splice site detection, where SpliceAI is considered state-of-the-art (J. Redmon et al. 2016). The best SegmentNT-30 kb model was compared with the specialized SpliceAI-10 kb model on detecting splice donor and acceptor nucleotides on a gene from a test set (EBF4) as shown in FIG. 13A. SegmentNT correctly predicts all exons and introns in addition to all splice sites, including the ones of the alternative exon at the gene start. When comparing both models it was observed that SpliceAI predicts all existent splice sites but overpredicts additional sites-red stars in FIG. 13A.

For a systematic comparison, SegmentNT and SpliceAI were evaluated in both SpliceAI's test set and SegmentNT's test set given their differences. Specifically, SpliceAI was trained and tested solely on pre-mRNA transcripts from protein-coding genes, without intergenic sequences, and with transcript sequences always in the respective positive strand. In contrast, SegmentNT training and test sets are more general and contain the whole DNA sequence of the respective chromosomes, including protein-coding genes and lncRNAs in both positive and negative orientation.

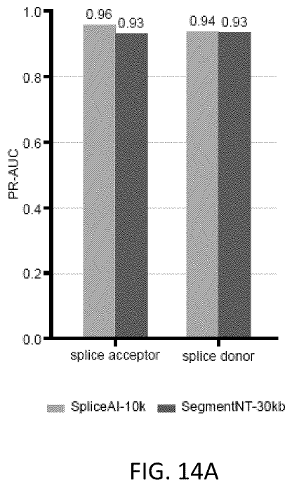

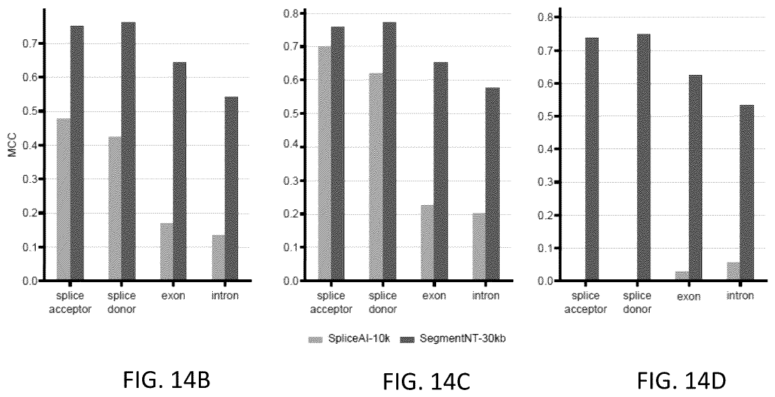

SegmentNT-30 kb achieves comparable performance to SpliceAI on SpliceAI's test set: PR-AUC for acceptor sites of 0.93 vs 0.96, and for donor sites of 0.93 vs 0.94, respectively as shown in FIG. 13B. The result is the same if using only 10 kb input sequences, the length used for SpliceAI training (PR-AUC acceptor: 0.92 vs 0.94, donor: 0.92 vs 0.87) as shown in FIG. 14A. On SegmentNT's whole genome test set, SegmentNT achieves substantially improved performance when considering all genes (acceptor: 0.75 vs 0.48, donor: 0.76 vs 0.42) or only the ones in the positive orientation (acceptor: 0.76 vs 0.70, donor: 0.77 vs 0.62) as shown in FIGS. 13C-13E. As expected, given its training data constraints, SpliceAI cannot predict splice sites when the gene is in the negative orientation, while SegmentNT maintains the same performance (acceptor: 0.74 vs 0.00, donor: 0.75 vs 0.00). Similar improvements were observed when considering 10 kb sequences as input as shown in FIGS. 14B-14D. Overall, SegmentNT accurately detects splice donor and acceptor sites in both strands in any given input DNA sequence.

Another difference to SpliceAI is that SegmentNT also predicts the position of exons and introns. This can only be achieved with SpliceAI by combining the splice donor and acceptor predictions a posteriori into exon and intron segments. SpliceAI was used to predict the position of exons and introns and compare with the segmentation predictions of SegmentNT. Here, SegmentNT also showed improved performance as shown in FIGS. 13C-13E.

This prediction of splice sites together with exon and intron segments by SegmentNT also allows for the direct prediction of potential transcript isoforms for a given DNA sequence. Given the accuracy of SegmentNT's predictions, the ability to evaluate the effect of sequence variants on isoform structures was tested. Data from an experimental saturation mutagenesis splicing assay of the exon 11 of the gene MST1R, flanked by constitutive exons 10 and 12 and respective introns, was used (data from S. Braun et al. 2018; see Methods). This dataset contains a library of almost 5,800 randomly mutated minigenes of ˜700nt, where for each minigene variant it was evaluated the splicing of the alternative exon 11 in the respective mRNA molecules. This data was used to test if SegmentNT could predict the impact of those sequence variants on the respective splicing and transcript isoforms.

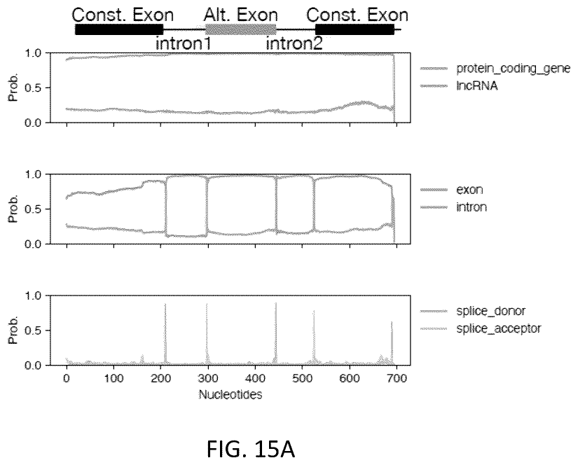

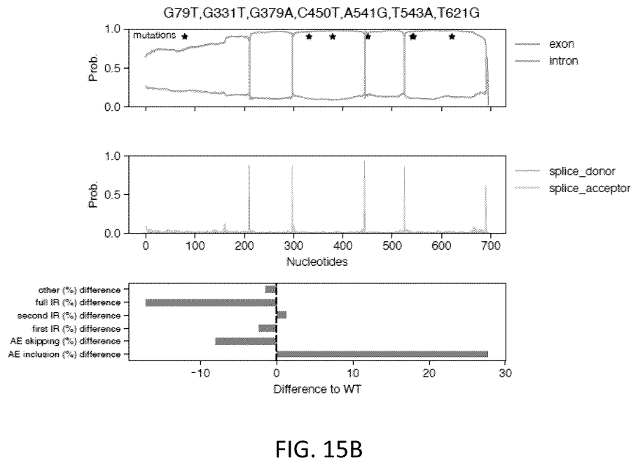

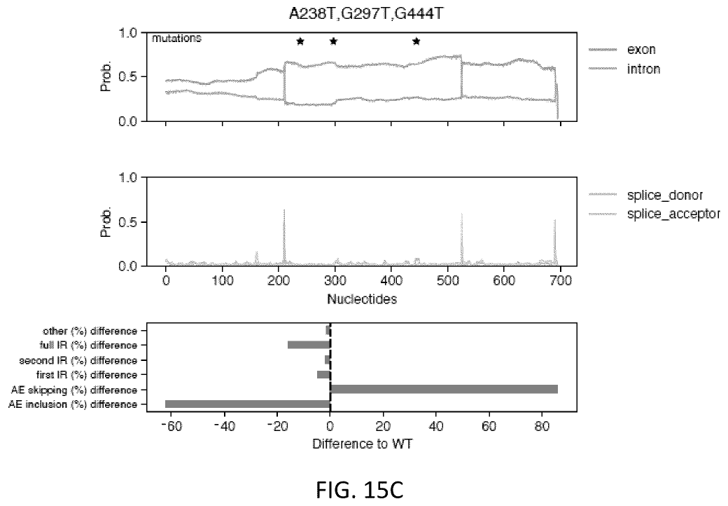

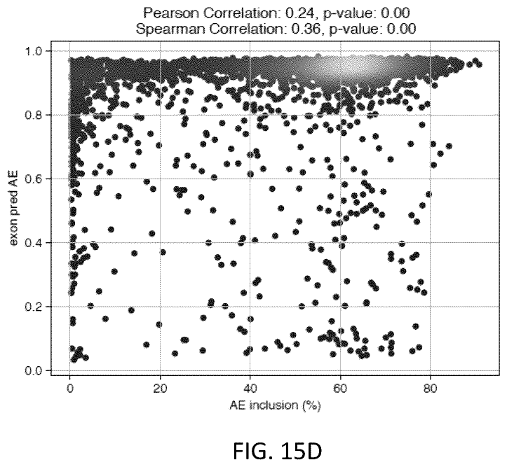

As a first check, SegmentNT correctly predicts this minigene as protein-coding, and the respective locations of all splice sites, the three exons and the two introns as shown in FIG. 15A. Sequence variants with different experimentally measured impacts in the minigene transcripts were further evaluated. FIG. 15B shows a minigene variant that leads to higher exon 11 inclusion, which is correctly predicted by SegmentNT-note the stronger exon prediction compared to the wildtype sequence, accompanied by stronger flanking intron and splice site predictions. FIG. 15C shows a minigene variant where the exon is skipped with high frequency. SegmentNT correctly predicts the loss of splice sites and of the respective exon, with higher prediction of an intron at its place. Systematic correlations across all minigene variants revealed a strong agreement between the exon predictions by SegmentNT and the inclusion of the alternative exon 11 (PCC: 0.24) as shown in FIG. 15D. These results show that the segmentation capabilities of SegmentNT can be used to predict complex gene rearrangements directly from the sequence, which should be a useful tool for the interpretation of sequence and structural variants that can affect gene regulation and disease.

E.vi. Zero-Shot Generalization of SegmentNT Across Species

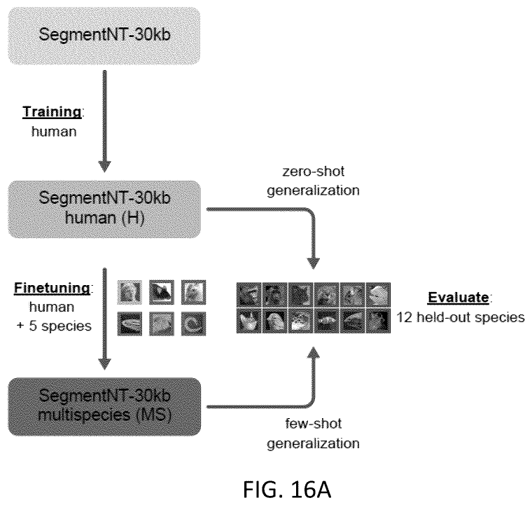

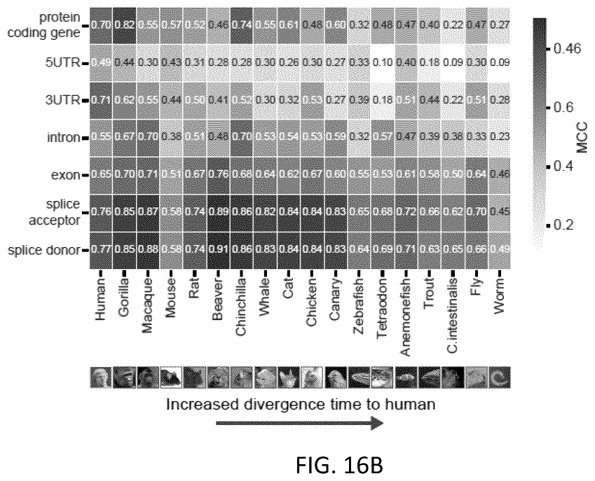

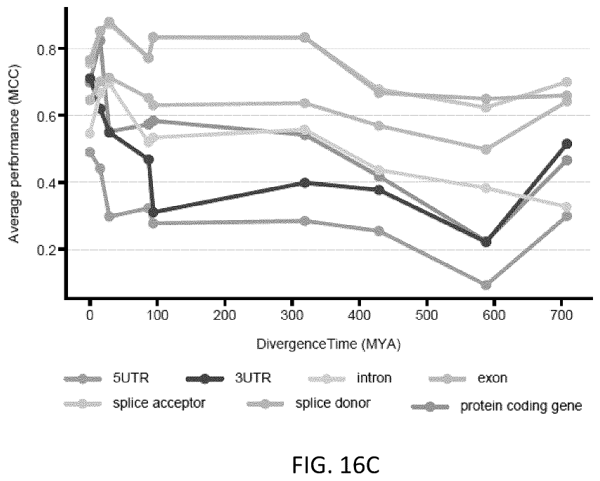

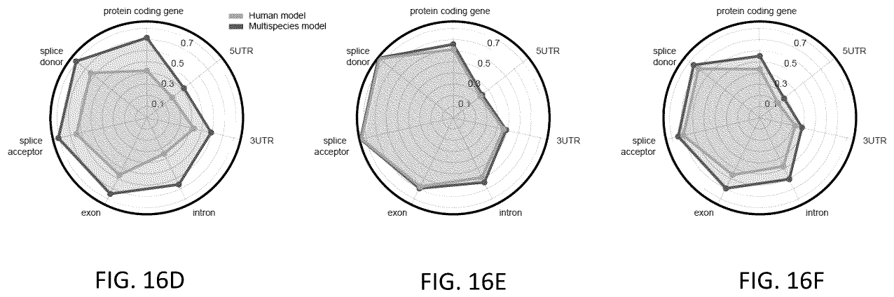

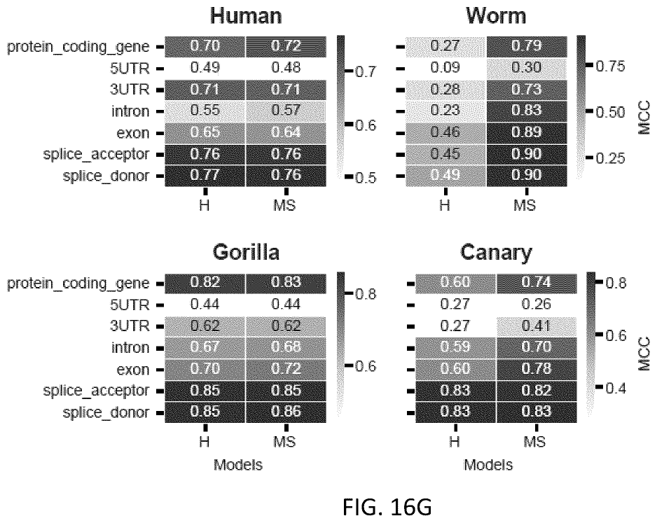

The ability of SegmentNT trained on human genomics elements to be generalize to other species was further explored as shown in FIG. 16A. Gene annotations for more distant, less-studied species are less accurate, while annotations of regulatory elements such as promoters and enhancers are very scarce. Thus, models that can predict these elements for different species hold great potential. In addition, comparison of predictions across species should provide insights about the evolutionary constraints of each element.

For this analysis, 17 additional species were selected and for each one a dataset of annotations was curated for the 7 main genomic elements available from Ensembl (F. J. Martin et al. 2023), namely protein-coding gene, 5′UTR, 3′UTR, intron, exon, splice acceptor and donor sites. This setup allows to evaluate the performance of the human model in each species on the 7 element types, while for the other 7 elements model predictions might be informative of potential regulatory regions. Similar to the human datasets, each dataset was split in train, validation and test chromosomes. The best model trained on the human 14 genomics elements, SegmentNT-30 kb, was selected and evaluated on each species test set.

High zero-shot performance was observed of the human SegmentNT-30 kb model across species for exon and splice sites, correlating with their high evolutionary conservation as shown in FIGS. 16B-16C. For the other elements the performance was good for related species like gorilla and macaque, but dropped for more evolutionary-distant animals. These results show that the SegmentNT-30 kb model can generalize to some extent to other species, but that the performance depends on the evolutionary distance of the genomics elements and species.

E.vii. Multispecies SegmentNT Model Shows Improved Species Generalization

Since gene elements have evolved and therefore their sequence determinants might differ between species, an additional, multispecies model (SegmentNT-30 kb-multispecies) was trained by finetuning the human SegmentNT-30 kb model on the genetic annotations of 5 selected species: mouse, chicken, fly, zebrafish and worm. The remaining 12 species were kept as held-out test set species for comparing the generalization capabilities of the human and multispecies models. Since most training species have limited annotation of regulatory elements, this multispecies model focused only on genomic elements and therefore it should not be used for the prediction of regulatory elements. The performance of the SegmentNT-30 kb-multispecies model improved quickly during finetuning, leveraging its knowledge of human elements. An improved performance was observed across species for the SegmentNT-30 kb-multispecies model over the human SegmentNT-30 kb model as shown in FIG. 16D, showing that gene elements diverged between species, and it is necessary to adjust the model accordingly.

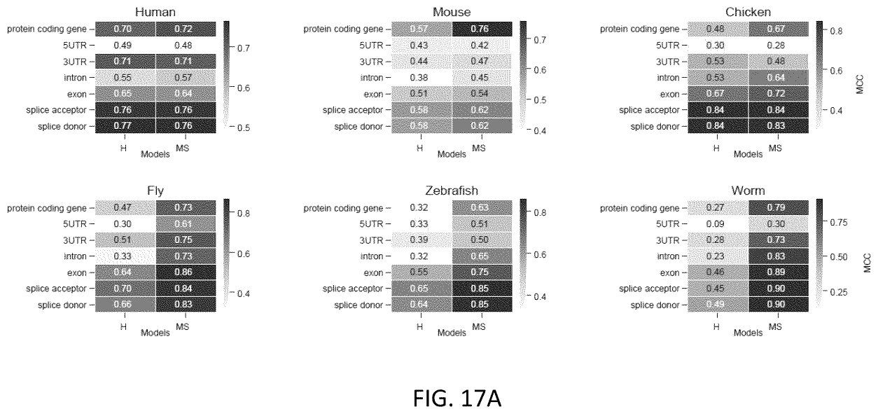

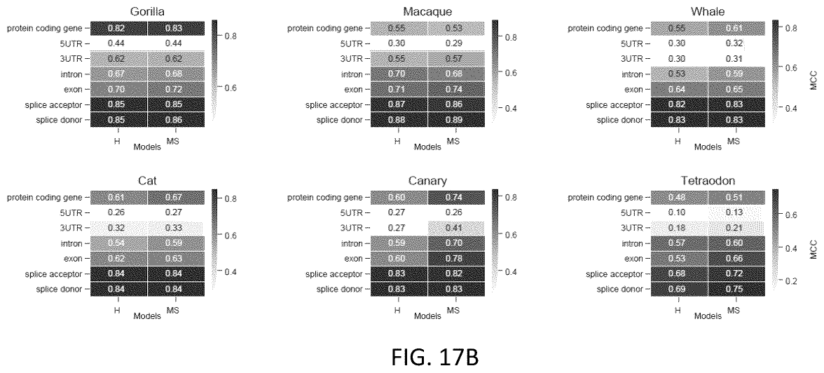

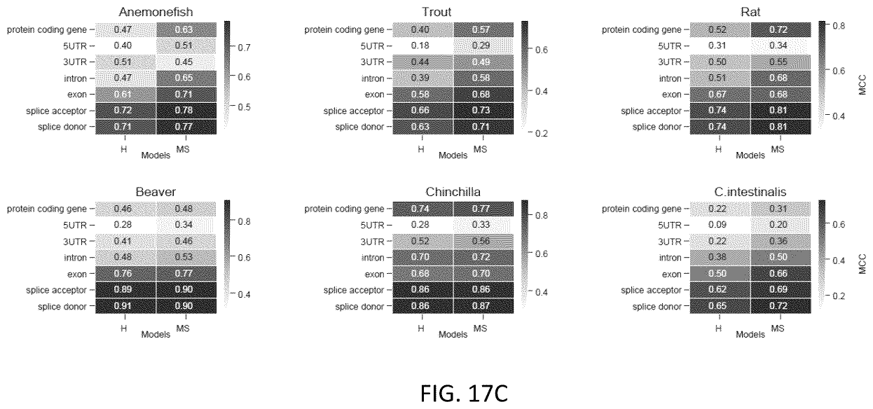

Finally, both human and multispecies SegmentNT-30 kb models were evaluated on the held-out set of 12 species, splitting them in two groups: 7 with an estimated divergence time from human of less than 100 million years (human-close species) and 5 more distant (more than 100 million years; human-distant; data from TimeTrec) as shown in FIGS. 11A-11C. The human model generalizes well for unseen species and showed better performance for human-close (average MCC of 0.62) than human-distant species (average MCC of 0.49) as shown in FIG. 16E-16F. SegmentNT-30 kb-multispecies demonstrated similarly good performance on human-close species (average MCC of 0.64) and improved performance on human-distant species (average MCC of 0.57) over the human model (0.49) as shown in FIGS. 5E-5F. This SegmentNT-30 kb-multispecies model is thus more general and can generalize to species not included in the training set as shown in FIGS. 16G and 17A-17C. Altogether, these results show that SegmentNT can be easily extended to additional genomics elements and species, which opens up promising new research directions to be explored in future work.

E.viii. Discussion

SegmentNT utilizes the DNA foundation model NT for predicting the location of several types of genomics elements in DNA sequences up to 50 kb at nucleotide resolution. Top performance for genomic elements, including splice sites, is demonstrated together with how each element depends on different context windows. For a given 50 kb sequence, SegmentNT makes 700,000 predictions at once allowing to annotate any input sequence in a very efficient way. SegmentNT trained on the human genome can already generalize to other species, but to make SegmentNT more broadly applicable to annotate sequences from different species a multispecies version was developed that improves generalization to unseen species.

SegmentNT provides strong evidence that DNA foundation models can tackle complex tasks in genomics at single-nucleotide resolution. Up until now, there is no consensus for the benefit of pretrained foundation models for genomics. There has been limited improvements on most tasks where these models have been evaluated on (Z. Zhou et al. 2023; H. Dalla-Torre et al. 2023; E. Nguyen et al. 2023; V. Fishman et al. 2023; F. I. Marin et al. 2023; E. Trop et al. 2024). SegmentNT successfully undertakes a more challenging task of segmenting genomic elements in DNA sequences at nucleotide resolution. The results show that the highest performance is achieved by combining a pre-trained NT and a segmentation U-Net head, when compared with applying such segmentation architectures directly from one-hot encoded DNA sequences. This is strong evidence for the value added by such pre-trained models and points to the need of expanding their applications and evaluations to more realistic tasks in genomics.

A current limitation of DNA foundation models before SegmentNT is their limited context length. NT was the pretrained model with the largest context length at its time, trained on sequences of up to 12 kb (H. Dalla-Torre et al. 2023). Since then, different approaches have been proposed to extend the context of such models, mostly by relying on novel state-space architectures to avoid the quadratic scaling of Transformers (E. Nguyen et al. 2023; E. Nguyen et al. 2024; Y. Schiff et al. 2024). A different approach is undertaken herein to extend the context of SegmentNT through context-length extrapolation in both training and evaluation phases, showing improved performance for sequences up to 50 kb (E. Trop, et al. 2024). The extension of the context of NT and SegmentNT models to longer sequences with efficient context-extension approaches is expected to yield further improvements for DNA segmentation tasks. Many techniques have recently emerged in fields like natural language processing that manage to increase the input length of Transformer models to process hundreds of thousands of tokens at a time (I. Betalgy et al. 2020; M. Zaheer et al. 2020; J. Ding et al. 2023; K. Guu et al. 2020). These approaches together with the new developments of state-space models provide promising avenues to build the next generation of models.

Lower performance was observed for the segmentation of promoter and enhancer regulatory elements compared with genic elements. Indeed, the sequence code of human regulatory elements is vastly more complex and unstructured, where for example the same element can encode different syntax in different cell types (J. Janssens et al. 2022). To account for some of this complexity split promoters and enhancers were split in tissue-invariant and tissue-specific classes each, observing different predictive performances between the groups. In future work, splitting promoters and enhancers by their specific cell types is expected to allow the model to learn the different cell type-specific regulatory codes thus improving the performance on regulatory element prediction.

An important result is the demonstration that SegmentNT trained on human genomics elements can generalize to unseen species. The generalization is stronger for splice sites and exons, likely due to their high conservation. In addition, reduced generalization was observed for species with longer divergence times to human. To improve the generalization to more distant species, a SegmentNT-multispecies model was developed that shows improved performance on unseen species. Thus, this model can be leveraged to annotate sequences up to 50 kb of any species de novo which should be useful to explore the genomes of less-characterized species.

Overall, the work has several direct applications. First, the finetuned DNA encoder within SegmentNT should provide stronger representations of human genomics elements and could be used to improve performance on downstream tasks (Z. Tang and P. K. Koo, 2024). Second, interpreting the representations learned by SegmentNT could reveal insights about the genome and its encoded information. Third, the accuracy of SegmentNT predictions can be leveraged to evaluate the impact of sequence variants on the different types of genomics elements, as was showed for splicing isoforms. Thanks to the extended sequence context and the prediction of several types of genomics elements, important applications are expected for the analysis of cancer genomes and their large structural variants. Fourth, SegmentNT-multispecies can be directly applicable to annotate and explore the genomes of different species. Fifth, SegmentNT's architecture can be easily applied to additional genomics annotations or nucleotide-level experimental data. Increasing the number of channels per nucleotides predicted by SegmentNT to include data coming from multiple experiments and biological processes should improve the transfer between tasks and lead to generalization in a way similar to the segment anything model for images (A. Kirillov et al. 2023). SegmentNT can be a useful tool for the genomics community and foster new developments in the understanding of the genome code.

E.ix. Methods: Genome Segmentation Model

SegmentNT is developed to approach genome segmentation problem as the segmentation of a sequence of N nucleotides (for example N=3,000 bp, 3 kb, or N=10,000 bp, 10 kb) by predicting a probability for each nucleotide to be part of one of K=14 elements: protein-coding gene, lncRNA, 5′UTR, 3′UTR, exon, intron, splice donor site, splice acceptor site, polyA signal, promoter tissue-invariant, promoter tissue-specific, enhancer tissue-invariant, enhancer tissue-specific or CTCF-bound.

### SegmentNT Architecture

Nucleotide Transformer (NT) can be used as a backbone for segmenting a sequence of nucleotides. SegmentNT uses the pre-trained NT-Multispecies-v2 (500M) model as DNA encoder to extract embeddings for each of the tokens yielded by a 6-mer tokenizer. N is the number of nucleotides in the DNA sequence and L the number of DNA tokens (with roughly L≈N/6). In order to segment the sequence, NT's original language model head is replaced by a 1-dimensional U-Net segmentation head (O. Ronneberger et al. 2015) made of 2 downsampling convolutional blocks and 2 upsampling convolutional blocks. Each of these blocks is made of 2 convolutional layers with 2,048 and 4,096 kernels respectively, and L/2 and L/4 sequence length. This accounts for 63 million parameters. The output of this layer is a N×K×2-dimensional vector which gives K probabilities for each nucleotide corresponding to the probability that the nucleotide is part of each type of genomics element. No further constraints are added on predictions such as the fact that one nucleotide belongs only to one element, and thus each nucleotide can be part of multiple elements.

### Model Training and Evaluation

The model is trained using Adam optimizer with a learning step lr=5e-5. A batch size of 256 is used and the SegmentNT-3 kb model is trained for 10.24B tokens, meaning a total of 20.48M sequences seen during training. The training was done on a cluster of 8 GPU H100 over 20 hours. The 10 kb, 20 kb and 30 kb models were initialized from the best checkpoint of the respective smaller model for faster adaptation to longer lengths. For example, SegmentNT-30 kb model was initialized with the best SegmentNT-20 kb checkpoint and finetuned for an additional 2.56B tokens (0.51M sequences). Focal loss (T.-Y. Lin et al. 2017) was used with y=2 which helps the model to focus on “harder” samples, i.e., the sparse nucleotides that belong to an element.

The dataset was split between train, validation and test sets by chromosome. Namely, chromosomes 20 and 21 are used for test, chromosome 22 is used for validation and the remaining are used for training. During training, sequences are randomly sampled in the genome with associated annotations. The sequences are kept in the validation and test sets fixed by using a sliding window of length N over the respective chromosomes. The validation set was used to monitor training and for early stopping while the test set was used to evaluate model performance. Matthews correlation coefficient (MCC) was used as a validation metric and selected the best checkpoint based on the average score across all 14 genomics elements. During evaluation and testing, for each sequence K probabilities per nucleotide were predicted, all predictions were concatenated across all sequences into a single array per element predicted, and MCC and Precision-Recall Area Under the Curve (PR-AUC) were computed for each genomic element over every nucleotide.

### Comparison of Different Architectures

SegmentNT is made of a DNA encoder, Nucleotide Transformer, and a 1-dimensional U-Net segmentation head, as described above. To evaluate the added value of using a pre-trained backbone encoder, the model was compared on the 3 kb sequences with (1) two versions of the U-Net segmentation head alone, with 63M and 252M parameters respectively, which take one-hot encoded DNA sequences as input instead of the embeddings outputted by the DNA encoder; and (2) a SegmentNT model whose encoder is initialized with random weights. Since when using one-hot encoded input sequences there is no aggregation of the base pairs into 6-mers, the input to the first convolutional layers of the U-Net model has a length of L=3,000 one-hot encoded base pairs instead of 500 token embeddings. As with the SegmentNT models, the training was monitored by validating on sequences from chromosome 22 and the best checkpoint was selected based on the highest average MCC score across the 14 types of elements. For the randomly initialized SegmentNT model, training was stopped before this criteria was met because the training took significantly longer time and the performance on most of the genomics elements had plateaued. The 63M and 252M U-Net models converged after 14.3M and 18.4M sequences respectively, just before the SegmentNT-3 kb model at 20.4M sequences. However, to reach this point, they take 12 hours and 36 hours, respectively, against 20 hours for SegmentNT-3 kb.

### Context Length Extension

Since the DNA encoder of SegmentNT is using rotary positional embeddings (RoPE) that have been trained on a maximum sequence length of 2,048 tokens, its performance degrades very quickly when inferring on longer sequences. Several previous works have suggested adaptations to RoPE to better handle evaluation or fine-tuning on longer sequences, such as using Position Interpolation (Kaiokendev, 2023; S. Chen et al. 2023) or “NTK-aware” scaled RoPE, 2023. More recently, B. Peng et al. 2023 formalized different methods and augmented them to propose a final adaptation of RoPE to unseen lengths called YaRN. After testing the different approaches, YaRN did not introduce improvements to extending Segment-NT lengths compared to simply using “NTK-aware” ROPE. Since the latter is lighter to implement it was decided to use it for extending the context of SegmentNT.

As described by B. Peng et al. 2023, with the hidden layer set of hidden neurons denoted by D, and a sequence of vectors x₁, . . . x_{L}ϵR^{|D|}, “NTK-aware” ROPE can be described by the following equation:

$${f}_{w}^{′} ( {x}_{m} , m , {θ}_{d} ) = f ( {x}_{m} , g ( m ) , h ( {θ}_{d} ) )$$

where d is the position along the embedding dimension, m is the position of the embedding in the sequence, f is the ROPE function (detailed in Eq. 1 of S. Chen et al. 2023), g (m)=m, h (θ_{d})=b′^{-2d/|D|}, b′=b.s^{|D|/(|D|-2)} and finally 2π/θ_{d}=2πb^{2d/|D|}. The rescaling factor s is computed as s=L′/L with L′ the extended context length and L the training context length, which for the NT-Multispecies-v2 (500M) is 2,048 tokens.

For SegmentNT models trained with “NTK-aware” ROPE, all sequences with length inferior to their training length are evaluated with the same rescaling factor that was used during the training. Concretely, SegmentNT-30 kb is trained with s=2.44, and therefore inference on a sequence smaller than 30,000 bp is done with s=2.44. When evaluated on a 50 kb sequence, the rescaling factor becomes s=4.07.

### Multi-Species Training

An additional, multispecies model (SegmentNT-10 kb-multispecies) was trained by finetuning the human SegmentNT-10 kb model on the annotations of five species together (mouse, chicken, fly, zebrafish and worm). The same model hyperparameters and training parameters were used. Since the different species have different genome sizes, examples from each dataset were balanced with the following weights: 5 for human, 4 for mouse, 2 for chicken, fly and zebrafish, and 1 for worm. Similar to the human dataset, the chromosomes of each species were split intro training, validation and test set.

E.x. Methods: Genome Annotation Data

### Human Genomics Elements

The human segmentation dataset of genomics elements was created from 14 types of elements, divided in gene elements (protein-coding genes, lncRNAs, 5′UTR, 3′UTR, exon, intron, splice acceptor and donor sites) and regulatory elements (polyA signal, tissue-invariant and tissue-specific promoters and enhancers, and CTCF-bound sites). The final segmentation dataset was created by overlapping all 14 elements with every DNA sequence of length N nucleotides. Sequences with Ns were removed.

The location of all gene elements and polyA signals were obtained from GENCODE (J. Harrow et al. 2012) V44 gene annotation. Annotations were filtered to exclude level 3 transcripts (automated annotation), so all training data was annotated by a human. Extract splice sites.py from HISAT2 (D. Kim et al. 2019) was used to extract respective intron and splice site annotations.

Promoter, enhancer and CTCF-bound sites were retrieved from ENCODE's SCREEN database (ENCODE Project Consortium, 2020). Distal and proximal enhancers were combined. Promoters and enhancers were split in tissue-invariant and tissue-specific based on the vocabulary from W. Meuleman et al. 2020. Enhancers or promoters overlapping regions classified as tissue-invariant were defined as that, while all other enhancers and promoters were defined as tissue-specific.

### Multi-Species Dataset

To create segmentation datasets for additional species only on the main gene elements were considered: protein-coding genes, 5′UTR, 3′UTR, exon, intron, splice acceptor and donor sites. Their annotations were obtained as described for the human dataset but retrieved from Ensembl databases. Five species were considered to train the multispecies model: mouse (mm10), chicken (galGal6), fly (dm6), zebrafish (danRer11) and worm (ce11). A held-out test set was created out of 12 species: gorilla (gorGor4), macaque (Mnem 1), rat (mRatBN7), beaver (can genome v1), chinchilla (ChiLan1), whale (ASM228892v3), cat (Felis catus 9), canary (SCA1), tetradon (T ET RAODON8), anemonefish (AmpOce1), trout (f SalT ru1) and Ciona intestinalis (KH). Evolutionary distance data was retrieved from Timetree of Life.

E.xi. Methods: Benchmarking for Regulatory Elements

### Sliding Nucleotide Transformer Finetuned Models

SegmentNT-10 kb was compared with a sliding window approach, where a binary classifier is used to predict the output probability for multiple sliding windows of the input 10 kb DNA sequence. This approach was applied for the segmentation of the two best classes of regulatory elements: promoter tissue-invariant and enhancer tissue-specific. As binary classifier the NT finetuned models were used on promoter and enhancer, respectively (H. Dalla-Torre et al. 2023). Sliding windows were created using a step size of 10 and the input size of the respective promoter (300 nt) and enhancer (200 nt) models. All inference times were calculated in a single A100 GPU.

Comparison with Enformer Zero-Shot Predictions

SegmentNT was compared with Enformer (Z. Avsec et al. 2021) for promoter predictions. For each 10 kb input sequence, the sequences were padded as requested by the model input dimensions and all Enformer predictions were computed at the original 128 bp bin resolution and the average over 7 selected ATAC-seq profiles was used for different human cell lines as quantitative score of regulatory activity. The PR-AUC metric was reported for the predictive value of this quantitative score to identify promoters at nucleotide resolution. All inference times were calculated in a single A100 GPU.

E.xii. Methods: Splicing Tasks
Comparison with SpliceAI

SegmentNT was compared with SpliceAI (K. Jaganathan et al. 2019) on both SpliceAI's test set and SegmentNT's test set given their different settings. The scripts were used that are available at the Illumina Basespace platform (G. Eraslan et al. 2019) to reproduce the testing dataset presented in SpliceAI for both 10 kb and 30 kb input sequences without additional context. This test set contains only mRNA sequences and all in the forward strand (i.e., for genes in the reverse strand, the sequence is reversed to have the gene in the forward orientation). Both models were also compared on the SegmentNT's 10 kb and 30 kb test sets, which contains all windows of the test chromosomes, including windows without genes or with genes in both the forward and reverse strand. Both PR-AUC and MCC were used as performance metrics.

### Sequence Variants and Transcript Isoforms

Data from an experimental saturation mutagenesis splicing assay of the exon 11 of the gene MST1R, flanked by constitutive exons 10 and 12 and respective introns (data from S. Braun, et al. 2018) was used. This dataset contains a library of almost 5,800 randomly mutated minigenes of ˜700 nt, where for each minigene variant it was evaluated the splicing of the alternative exon 11 in the respective mRNA molecules. This data was used to test if SegmentNT could predict the impact of those sequence variants on the respective splicing and transcript isoforms. Only on minigene variants composed of combinations of single-nucleotide mutations were considered. All 14 genomics elements in the wildtype minigene sequence and all minigene variants were predicted. For a systematic comparison, the predicted exon score for the region of the alternative exon 11 was compared with the experimentally measured exon inclusion scores.

### F. Example 2: Detection of Alternative Splicing Events with SegmentNT

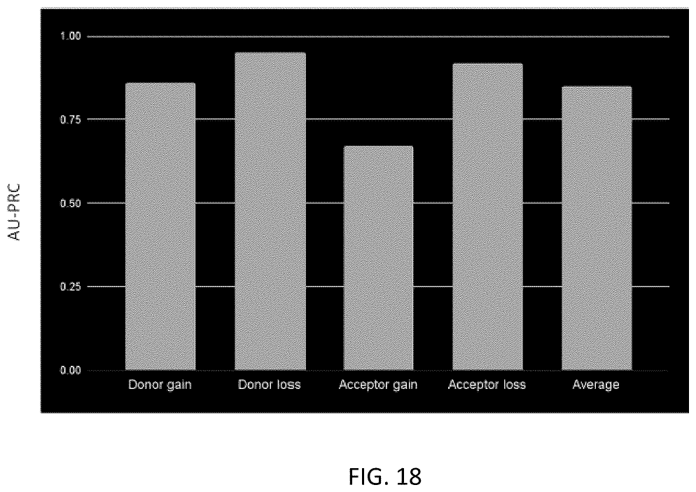

This example describes an exemplary use of certain embodiments of genomic element segmentation technologies described herein to predict alternative splicing events in cancer data. Alternative splicing events can disrupt protein production and cancer pathways, leading to cancer development. In this example, the SegmentNT model described in Example 1 above was fine-tuned to identify neoantigens from alternative splicing events, which, for example, may be used as potential targets for personalized cancer vaccines. FIG. 18 shows performance data (area under precision-recall curve) for predicting loss and/or gain of fragments in donor and/or acceptor sites in cancer data. Data used for evaluation was obtained from Y. Shiraishi et al. “A comprehensive characterization of cis-acting splicing-associated variants in human cancer” 2018. As shown, after finetuning, SegmentNT can predict very accurately alternative splicing in cancer data.

### G. Example 3: Utilizing Different Foundation Models as Encoders

This example describes and demonstrates performance of three different implementations of genomic element segmentation technologies of the present disclosure, demonstrating the use of different types of foundation models as encoders.

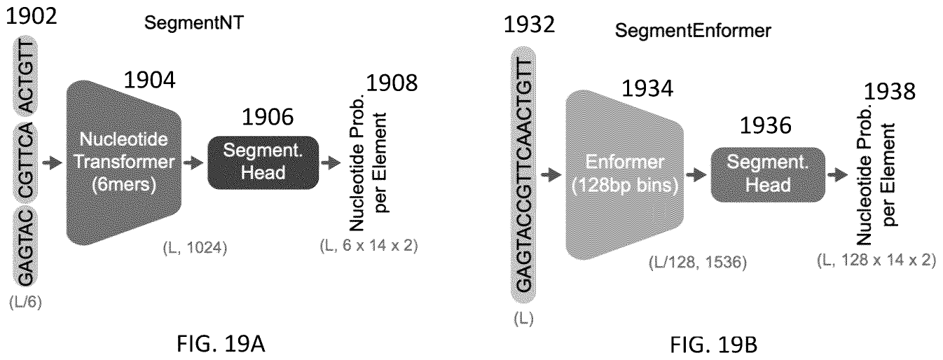

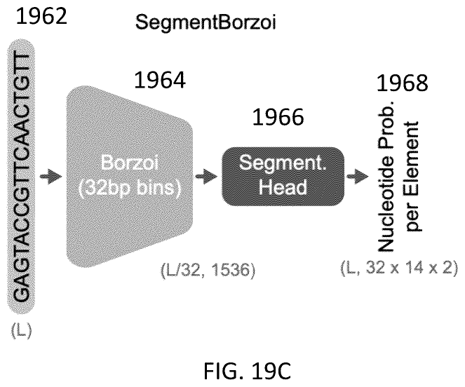

As shown in FIGS. 19A-19C, three different approaches—each using a different foundation model as an encoder in combination with a segmentation head—were implemented and evaluated in this example.

FIG. 19A illustrates a first approach, which corresponds to the SegmentNT (30 kb) model described in Example 1, whereby a language model-based encoder-namely, the Nucleotide Transformer (NT) model described in Dalla-Torre et al., “The Nucleotide Transformer: Building and Evaluating Robust Foundation Models for Human Genomics,” bioRxiv, 2023 is used as an encoder 1904, to generate embeddings that are passed as input to segmentation head 1906, which, in turn, generates outputs 1908 comprising a set of (K=14) likelihood values for each nucleotide in input sequence 1902 (shown as the 6-mer tokens). For each particular nucleotide, each likelihood value of the set represents a probability that the particular nucleotide is part a particular one of one of the K=14 genomic elements described in Example 1. The output of immediately following the segmentation head is a set of N×K×2 values for each token, where N=6 and K=14. Two values are output for each nucleotide and genomic element combination, corresponding to (p, 1-p), where p is the probability—this is an implementation choice, whereby two logits are transformed via a softmax into (p, 1-p), but other implementations are possible, such as returning one value, p. As shown in FIGS. 19B and 19C, and described below, the two other encoder approaches use a similar, N×K×2 format.

FIGS. 19B and 19C illustrate two other approaches, which utilize other foundation models as encoders for generating embeddings to be passed as input to a segmentation head. One approach, illustrated in FIG. 19B utilizes a model referred to as “Enformer” as an encoder and another, illustrated in FIG. 19C, utilizes a model referred to as “Borzoi” as an encoder. The Enformer and Borzoi models are described in detail in Avsec et al., “Effective gene expression prediction from sequence by integrating long-range interactions,” Nature methods, vol. 18, no. 10, pp. 1196-1203, 2021 and Linder et al., “Predicting RNA-seq coverage from DNA sequence as a unifying model of gene regulation,” bioRxiv, pp. 2023-08, 2023, respectively, the content of each of which is hereby incorporated by reference herein in their entirety.

These models differ from the NT model in two respects. First, they (Enformer and Borzoi) use a mixture of convolution and attention (e.g., via transformer blocks) and second, they are pre-trained via a supervised learning approach on example genomics tracks (e.g., mainly chromatin accessibility and gene expression tracks in multiple tissues), whereas the NT model is pre-trained in a self-supervised fashion on genomes. Without wishing to be bound to any particular theory, in certain implementations, by incorporating convolutional blocks in their architectures, Enformer and/or Borzoi style encoders allow for longer inputs-196 kb and 524 kbp for Enformer and Borzoi, respectively, than a purely transformer-based approach. Additionally or alternatively, while the NT model is pre-trained with self-supervised learning on genomes. Accordingly, they provide examples of architectures that incorporate convolutional layers, and utilize supervised training, thereby offering the potential to process larger sequences and/or the ability to extract different features in comparison with the NT model-based encoder shown in FIG. 19A.

As shown in FIG. 19B, the Enformer-based implementation (referred to as “SegmentEnformer”) received a sequence of length L (196 kb) as input 1932 and used an Enformer-based encoder 1934 (e.g., an encoder portion of an Enformer model), which comprises a tower of convolutional blocks followed by a transformer block, to generate an embedding vector for each 128 bp section of the input sequence (e.g., 128 bp bins). The embeddings were extracted by removing all the layers after the transformer portion of the Enformer architecture and providing the resultant intermediate representing as an input embedding to segmentation head 1936. As shown in FIG. 19B, the resultant embedding representation comprises L/128 embedding vectors, each having a length of 1536. Segmentation head 1936, was, as described above in Example 1, a 1D U-net architecture. For the Enformer-based architecture, segmentation head 1936 head resamples to 128 nucleotides per position (as opposed to 6, the token size, in the NT-based model), and generates sets of likelihood values for each nucleotide, as described above.

Turning to FIG. 19C, the Borzoi-based implementation (referred to as “SegmentBorzoi”) receives as sequence of length L (524 kbp) as input 1962 and uses a Borzoi-based encoder 1964 (e.g., an encoder portion of a Borzoi model), which comprises a transformer block placed in between two sets of convolutional layers that, respectively, down-sample input and up-sample output from the transformer block. By removing only the very final layer—an output head used to generate genomic track predictions in the stand-alone Borzoi implementation, an embedding vector for each 32 bp section of the input sequence (e.g., 32 bp bins) was obtained. As with the other models, the resultant embeddings were used as input to a 1D U-net segmentation head 1966, which, here, resampled to 32 nucleotides per position, thereby generating likelihood values for each nucleotide.

Each of the three models—SegmentNT, SegmentEnformer, and SegmentBorzoi, as shown in FIGS. 19A-19C, were fine-tuned on the same data with the same hyperparameters (those described above in Example 1 with respect to the implementation of the SegmentNT model). The 30 kb implementation of the SegmentNT model described above in Example 1 was used for comparison with the SegmentEnformer, and SegmentBorzoi models.

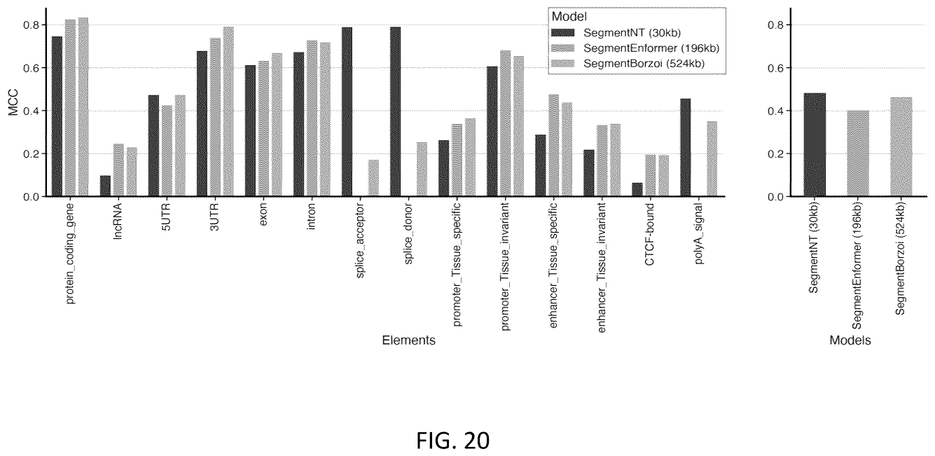

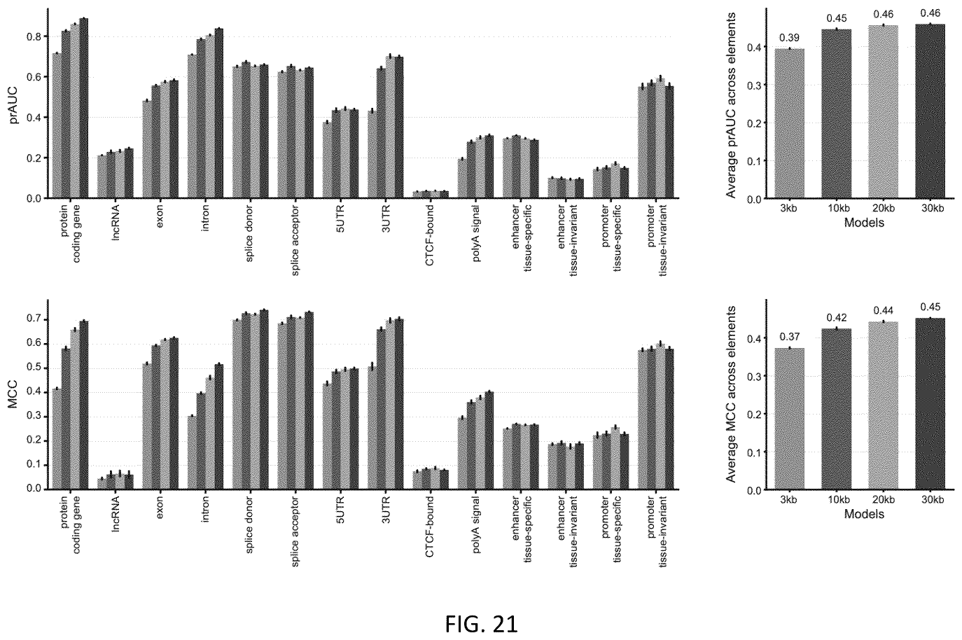

Turning to FIG. 20, once trained, each of the three models was evaluated in terms of performance in classifying each of the 14 genomic elements described in Example 1, above. FIG. 20 plots the Mathews Correlation Coefficient (MCC) values for the three models on each of the 14 genomic element segmentation tasks (left bar chart) along with overall performance (averaged across all 14 tasks) for each model (right bar chart). The Enformer and Borzoi-based models show improved performance on all elements except splice sites and polyA. Without wishing to be bound to any particular theory, this performance improvement is believed to result from the longer context-196 kb and 524 kb for SegmentEnformer and SegmentBorzoi, respectively, versus 30 kb for SegmentNT-resulting from the inclusion of convolutional layers in the Enformer and Borzoi-based models, along with, additionally or alternatively, the additional information incorporated in those two models by virtue of their supervised training approach.

Accordingly, this example demonstrates that encoders based on various types of foundational models—e.g., machine learning models having been previously trained on large quantities of data—may be used in various implementations and embodiments of the genomic element segmentation technologies of the present disclosure.

### H. Example 4: Extended Performance Evaluation of SegmentNT

This example describes and provides additional performance metrics for the SegmentNT model described above, in Example 1.

FIG. 21 shows a set of graphs evaluating performance of four different size SegmentNT models across the 14 genomic elements described in Example 1. The upper plots measure performance in terms of area under the precision recall curve (prAUC), while the bottom plots measure performance via the Mathews Correlation Coefficient (MCC) described above in Example 1, e.g., shown in FIG. 11A and FIG. 11B.

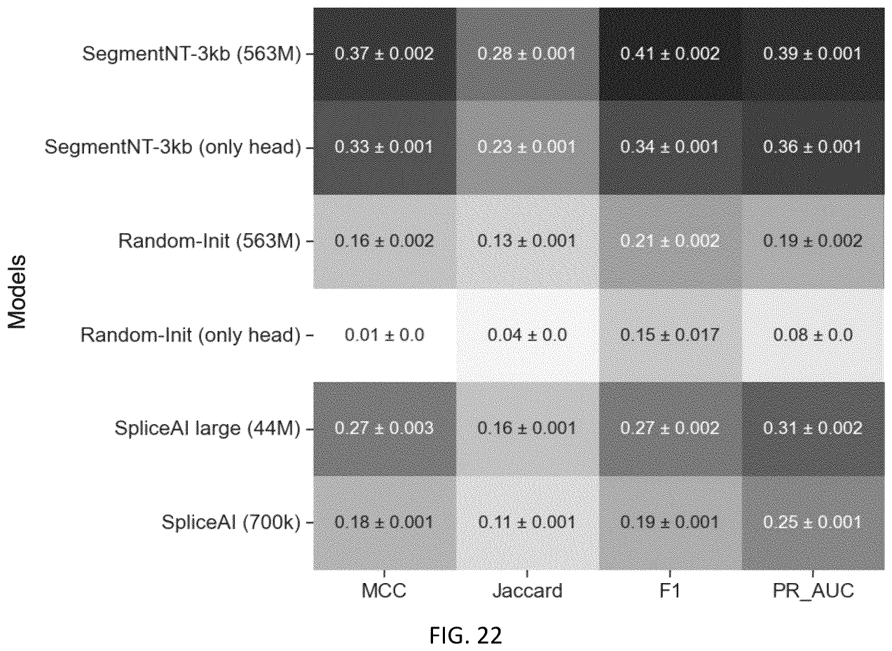

FIG. 22 shows performance of different baselines and ablations on the 3 kb dataset described above in Example 1. Performance is measured in terms of MCC, Jaccard, F1 score, and prAUC (column-wise). Different models and baselines are listed row-wise, with “only head” denoting versions of the SegmentNT model with only the segmentation head, and Random-Init referring to a SegmentNT model where the NT encoder has not been pre-trained (with self-supervised learning), but, instead, is randomly initialized (e.g., as a sanity check baseline). The two SpliceAI model rows provide values for a randomly initialized baseline using a similar neural net architecture to the spliceAI model described in (J. Redmon et al. 2016).

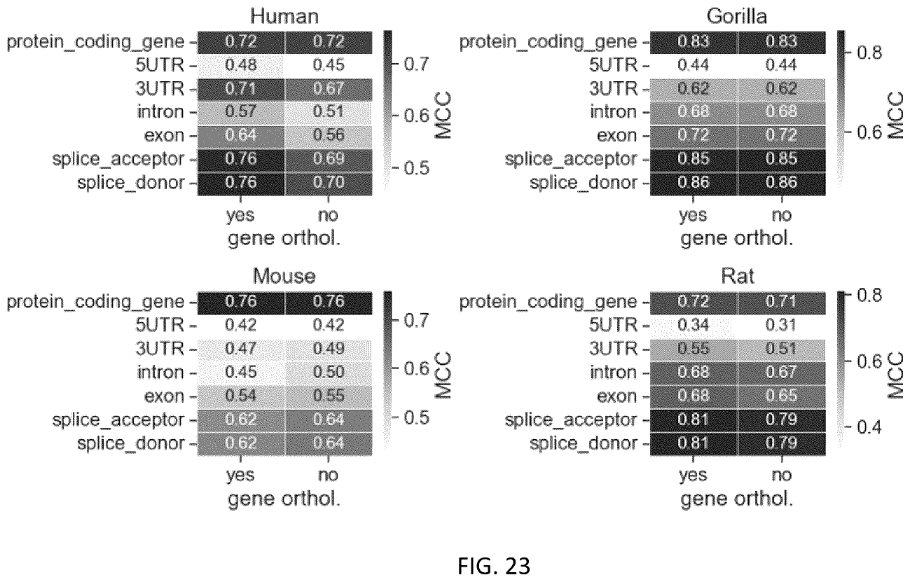

FIG. 23 shows experiments assessing model performance on test datasets where orthologous genes—certain genes present in human genomes that are also present in other species genomes—are removed, to further demonstrate that the models generalize. As shown in the figure, removing the orthologous genes does not significantly impact performance.

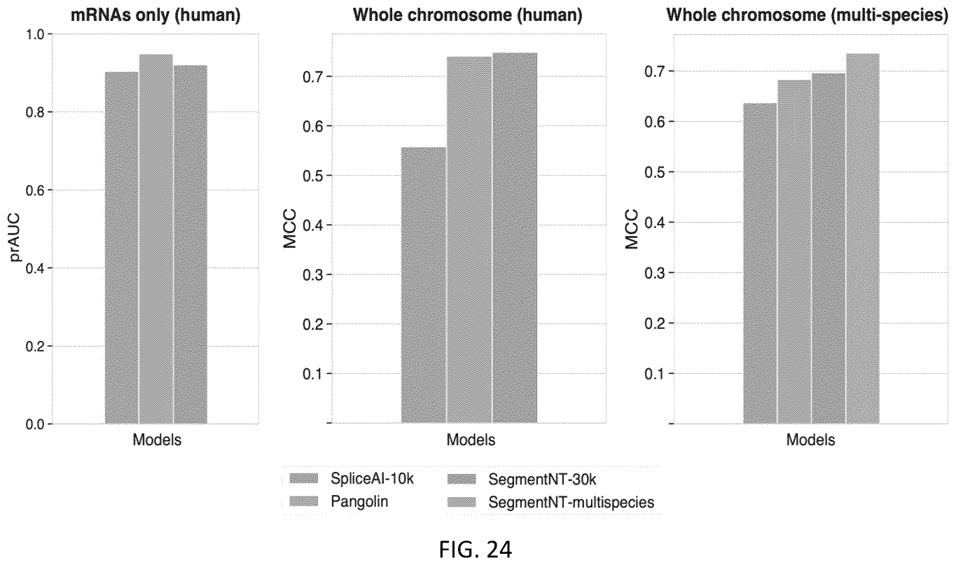

FIG. 24 evaluates performance of SegmentNT against SpliceAI and Pangolin on three datasets corresponding to (1) human annotations over mRNAs only, (2) human annotations over full chromosomes and (3) 23-species annotations over full chromosomes.

Accordingly, this example provides additional performance metrics demonstrating improvements offered via the nucleotide sequence annotation technologies of the present disclosure.

### EQUIVALENTS

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

### CERTAIN REFERENCES

G. Eraslan, Z. Avsec, J. Gagneur, and F. J. Theis, “Deep learning: new computational modelling techniques for genomics,” Nature Reviews Genetics, vol. 20, no. 7, pp. 389-403, 2019.
T. Yue, Y. Wang, L. Zhang, C. Gu, H. Xue, W. Wang, Q. Lyu, and Y. Dun, “Deep learning for genomics: From early neural nets to modern large language models,” International Journal of Molecular Sciences, vol. 24, no. 21, p. 15858, 2023.
J. Zhou and O. G. Troyanskaya, “Predicting effects of noncoding variants with deep learning-based sequence model,” Nature methods, vol. 12, no. 10, pp. 931-934, 2015.
B. Alipanahi, A. Delong, M. T. Weirauch, and B. J. Frey, “Predicting the sequence specificities of dna- and rna-binding proteins by deep learning,” Nature Biotechnology, vol. 33, p. 831-838, 2015.
D. R. Kelley, J. Snoek, and J. L. Rinn, “Basset: learning the regulatory code of the accessible genome with deep convolutional neural networks,” Genome research, vol. 26, no. 7, pp. 990-999, 2016.
D. R. Kelley, Y. A. Reshef, M. Bileschi, D. Belanger, C. Y. McLean, and J. Snoek, “Sequential regulatory activity prediction across chromosomes with convolutional neural networks,” Genome Research, vol. 28, pp. 739-750, March 2018.
D. R. Kelley, “Cross-species regulatory sequence activity prediction,” PLOS Computational Biology, vol. 16, p. e1008050, July 2020.
Z. Avsec, M. Weilert, A. Shrikumar, S. Krueger, A. Alexandari, K. Dalal, R. Fropf, C. McAnany, J. Gagneur, A. Kundaje, et al., “Base-resolution models of transcription-factor binding reveal soft motif syntax,” Nature Genetics, vol. 53, no. 3, pp. 354-366, 2021.
B. P. de Almeida, F. Reiter, M. Pagani, and A. Stark, “Deepstarr predicts enhancer activity from dna sequence and enables the de novo design of synthetic enhancers,” Nature Genetics, vol. 54, no. 5, pp. 613-624, 2022.
J. Linder, S. E. Koplik, A. Kundaje, and G. Seelig, “Deciphering the impact of genetic variation on human polyadenylation using aparent2,” Genome Biology, vol. 23, p. 232, 2022.
V. Agarwal and D. R. Kelley, “The genetic and biochemical determinants of mrna degradation rates in mammals,” Genome Biology, vol. 23, p. 245, 2022.
F. Stiehler, M. Steinborn, S. Scholz, D. Dey, A. P. Weber, and A. K. Denton, “Helixer: cross-species gene annotation of large eukaryotic genomes using deep learning,” Bioinformatics, vol. 36, no. 22-23, pp. 5291-5298, 2020.
M. R. Amin, A. Yurovsky, Y. Tian, and S. Skiena, “Deepannotator: genome annotation with deep learning,” in Proceedings of the 2018 ACM International conference on bioinformatics, computational biology, and health informatics, pp. 254-259, 2018.
D. Quang and X. Xie, “Danq: a hybrid convolutional and recurrent deep neural network for quantifying the function of dna sequences,” Nucleic acids research, vol. 44, no. 11, pp. e107-e107, 2016.
L. Minnoye, I. I. Taskiran, D. Mauduit, M. Fazio, L. V. Aerschot, G. Hulselmans, V. Christiaens, S. Makhzami, M. Seltenhammer, P. Karras, A. Primot, E. Cadieu, E. van Rooijen, J.-C. Marine, G. Egidy, G. E. Ghanem, L. Zon, J. Wouters, and S. Aerts, “Cross-species analysis of enhancer logic using deep learning,” Genome Research, vol. 30, pp. 1815-1834, 2020.
Z. Avsec, V. Agarwal, D. Visentin, J. R. Ledsam, A. Grabska-Barwinska, K. R. Taylor, Y. Assael, J. Jumper, P. Kohli, and D. R. Kelley, “Effective gene expression prediction from sequence by integrating long-range interactions,” Nature methods, vol. 18, no. 10, pp. 1196-1203, 2021.
J. Linder, D. Srivastava, H. Yuan, V. Agarwal, and D. R. Kelley, “Predicting rna-seq coverage from dna sequence as a unifying model of gene regulation,” bioRxiv, pp. 2023-08, 2023.
M. Oubounyt, Z. Louadi, H. Tayara, and K. T. Chong, “Deepromoter: robust promoter predictor using deep learning,” Frontiers in genetics, vol. 10, p. 286, 2019.
K. M. Chen, A. K. Wong, O. G. Troyanskaya, and J. Zhou, “A sequence-based global map of regulatory activity for deciphering human genetics,” Nature genetics, vol. 54, no. 7, pp. 940-949, 2022.
Y. Ji, Z. Zhou, H. Liu, and R. V. Davuluri, “Dnabert: pre-trained bidirectional encoder representations from transformers model for dna-language in genome,” Bioinformatics, vol. 37, no. 15, pp. 2112-2120, 2021.
Z. Zhou, Y. Ji, W. Li, P. Dutta, R. Davuluri, and H. Liu, “Dnabert-2: Efficient foundation model and benchmark for multi-species genome,” arXiv preprint arXiv: 2306.15006, 2023.
H. Dalla-Torre, L. Gonzalez, J. Mendoza-Revilla, N. L. Carranza, A. H. Grzywaczewski, F. Oteri, C. Dallago, E. Trop, B. P. de Almeida, H. Sirelkhatim, G. Richard, M. Skwark, K. Beguir, M. Lopez, and T. Pierrot, “The nucleotide transformer: Building and evaluating robust foundation models for human genomics,” bioRxiv, 2023.
E. Nguyen, M. Poli, M. Faizi, A. Thomas, C. Birch-Sykes, M. Wornow, A. Patel, C. Rabideau, S. Massaroli, Y. Bengio, et al., “Hyenadna: Long-range genomic sequence modeling at single nucleotide resolution,” arXiv preprint arXiv: 2306.15794, 2023.
G. Benegas, S. S. Batra, and Y. S. Song, “Dna language models are powerful predictors of genome-wide variant effects,” Proceedings of the National Academy of Sciences, vol. 120, no. 44, p. e2311219120, 2023.
V. Fishman, Y. Kuratov, M. Petrov, A. Shmelev, D. Shepelin, N. Chekanov, O. Kardymon, and M. Burtsev, “Gena-Im: A family of open-source foundational models for long dna sequences,” bioRxiv, pp. 2023-06, 2023.
J. Devlin, M.-W. Chang, K. Lee, and K. Toutanova, “Bert: Pre-training of deep bidirectional transformers for language understanding,” arXiv preprint arXiv: 1810.04805, 2018.
A. Radford, J. Wu, R. Child, D. Luan, D. Amodei, I. Sutskever, et al., “Language models are unsupervised multitask learners,” OpenAI blog, vol. 1, no. 8, p. 9, 2019.
H. Bao, L. Dong, S. Piao, and F. Wei, “Beit: Bert pre-training of image transformers,” 2022.
S. Gidaris, P. Singh, and N. Komodakis, “Unsupervised representation learning by predicting image rotations,” in International Conference on Learning Representations, 2018.
A. Radford, J. W. Kim, C. Hallacy, A. Ramesh, G. Goh, S. Agarwal, G. Sastry, A. Askell, P. Mishkin, J. Clark, G. Krueger, and I. Sutskever, “Learning transferable visual models from natural language supervision,” 2021.
A. E. Trevino, F. Muller, J. Andersen, L. Sundaram, A. Kathiria, A. Shcherbina, K. Farh, H. Y. Chang, A. M. Pasca, A. Kundaje, et al., “Chromatin and gene-regulatory dynamics of the developing human cerebral cortex at single-cell resolution,” Cell, vol. 184, no. 19, pp. 5053-5069, 2021.
S. Nair, M. Ameen, L. Sundaram, A. Pampari, J. Schreiber, A. Balsubramani, Y. X. Wang, D. Burns, H. M. Blau, I. Karakikes, K. C. Wang, and A. Kundaje, “Transcription factor stoichiometry, motif affinity and syntax regulate single-cell chromatin dynamics during fibroblast reprogramming to pluripotency,” bioRxiv, 2023.
O. Ronneberger, P. Fischer, and T. Brox, “U-net: Convolutional networks for biomedical image segmentation,” in Medical Image Computing and Computer-Assisted Intervention-MICCAI 2015: 18th International Conference, Munich, Germany, Oct. 5-9, 2015, Proceedings, Part III 18, pp. 234-241, Springer, 2015.
T.-Y. Lin, P. Goyal, R. Girshick, K. He, and P. Dollar, “Focal loss for dense object detection,” in Proceedings of the IEEE international conference on computer vision, pp. 2980-2988, 2017.
J. Redmon, S. Divvala, R. Girshick, and A. Farhadi, “You only look once: Unified, real-time object detection,” in Proceedings of the IEEE conference on computer vision and pattern recognition, pp. 779-788, 2016.
K. Jaganathan, S. K. Panagiotopoulou, J. F. McRae, S. F. Darbandi, D. Knowles, Y. I. Li, J. A. Kosmicki, J. Arbelaez, W. Cui, G. B. Schwartz, et al., “Predicting splicing from primary sequence with deep learning,” Cell, vol. 176, no. 3, pp. 535-548, 2019.
T. Zeng and Y. I. Li, “Predicting rna splicing from dna sequence using pangolin,” Genome biology, vol. 23, no. 1, pp. 1-18, 2022.
J. Harrow, A. Frankish, J. M. Gonzalez, E. Tapanari, M. Diekhans, F. Kokocinski, B. L. Aken, D. Barrell, A. Zadissa, S. Searle, I. Barnes, A. Bignell, V. Boychenko, T. Hunt, M. Kay, G.Mukherjee, J. Rajan, G. Despacio-Reyes, G. Saunders, C. Steward, R. Harte, M. Lin, C. Howald, A. Tanzer, T. Derrien, J. Chrast, N. Walters, S. Balasubramanian, B. Pei, M. Tress, J. M. Rodriguez, I. Ezkurdia, J. van Baren, M. Brent, D. Haussler, M. Kellis, A. Valencia, A. Reymond, M. Gerstein, R. Guigo, and T. J. Hubbard, “Gencode: The reference human genome annotation for the encode project,” Genome Research, vol. 22, pp. 1760-1774, 2012.
The ENCODE Project Consortium, “An integrated encyclopedia of dna elements in the human genome,” Nature, vol. 489, no. 7414, pp. 57-74, 2012.
The ENCODE Project Consortium, “Expanded encyclopedias of dna elements in the human and mouse genomes,” Nature, vol. 583, no. 7818, p. 699-710, 2020.
J. Su, Y. Lu, S. Pan, A. Murtadha, B. Wen, and Y. Liu, “Roformer: Enhanced transformer with rotary position embedding,” arXiv preprint arXiv: 2104.09864, 2021.
B. Peng, J. Quesnelle, H. Fan, and E. Shippole, “Yarn: Efficient context window extension of large language models,” arXiv preprint arXiv: 2309.00071, 2023.
E. Trop, C.-H. Kao, M. Polen, Y. Schiff, B. P. de Almeida, A. Gokaslan, T. Pierrot, and V. Kuleshov, “Advancing dna language models: The genomics long-range benchmark,” LLMs4Bio AAAIWorkshop 2024, 2024.
Y. Schiff, C.-H. Kao, A. Gokaslan, T. Dao, A. Gu, and V. Kuleshov, “Caduceus: Bi-directional equivariant long-range dna sequence modeling,” 2024.
S. Braun, M. Enculescu, S. T. Setty, M. Cortes-Lopez, B. P. de Almeida, F. R. Sutandy, L. Schulz, A. Busch, M. Seiler, S. Ebersberger, et al., “Decoding a cancer-relevant splicing decision in the ron proto-oncogene using high-throughput mutagenesis,” Nature communications, vol. 9, no. 1, p. 3315, 2018.
F. J. Martin, M. R. Amode, A. Aneja, O. Austine-Orimoloye, A. G. Azov, I. Barnes, A. Becker, R. Bennett, A. Berry, J. Bhai, et al., “Ensembl 2023,” Nucleic acids research, vol. 51, no. D1, pp. D933-D941, 2023.
F. I. Marin, F. Teufel, M. Horrender, D. Madsen, D. Pultz, O. Winther, and W. Boomsma, “Bend: Benchmarking dna language models on biologically meaningful tasks,” arXiv preprint arXiv: 2311.12570, 2023.
E. Nguyen, M. Poli, M. G. Durrant, A. W. Thomas, B. Kang, J. Sullivan, M. Y. Ng, A. Lewis, A. Patel, A. Lou, S. Ermon, S. A. Baccus, T. Hernandez-Boussard, C. Re, P. D. Hsu, and B. L. Hie, “Sequence modeling and design from molecular to genome scale with evo,” bioRxiv, 2024.
I. Beltagy, M. E. Peters, and A. Cohan, “Longformer: The long-document transformer,” arXiv preprint arXiv: 2004.05150, 2020.
M. Zaheer, G. Guruganesh, K. A. Dubey, J. Ainslie, C. Alberti, S. Ontanon, P. Pham, A. Ravula, Q. Wang, L. Yang, et al., “Big bird: Transformers for longer sequences,” Advances in Neural Information Processing Systems, vol. 33, pp. 17283-17297, 2020.
J. Ding, S. Ma, L. Dong, X. Zhang, S. Huang, W. Wang, N. Zheng, and F. Wei, “Longnet: Scaling transformers to 1,000,000,000 tokens,” 2023.
K. Guu, K. Lee, Z. Tung, P. Pasupat, and M.-W. Chang, “Realm: Retrieval-augmented language model pre-training,” 2020.
J. Janssens, S. Aibar, I. I. Taskiran, J. N. Ismail, A. E. Gomez, G. Aughey, K. I. Spanier, F. V. D. Rop, C. B. Gonzalez-Blas, M. Dionne, K. Grimes, X. J. Quan, D. Papasokrati, G. Hulselmans, S. Makhzami, M. D. Waegeneer, V. Christiaens, T. Southall, and S. Aerts, “Decoding gene regulation in the fly brain,” Nature, vol. 601, no. 7894, pp. 630-636, 2022.
Z. Tang and P. K. Koo, “Evaluating the representational power of pre-trained dna language models for regulatory genomics,” bioRxiv, 2024.
A. Kirillov, E. Mintun, N. Ravi, H. Mao, C. Rolland, L. Gustafson, T. Xiao, S. Whitehead, A. C. Berg, W.-Y. Lo, P. Dollar, and R. Girshick, “Segment anything,” 2023.
S. Chen, S. Wong, L. Chen, and Y. Tian, “Extending context window of large language models via positional interpolation,” arXiv preprint arXiv: 2306.15595, 2023.
Kaiokendev.github.io, “Things I'm learning while training superhot.” https://kaiokendev.github.io/til #extending-context-to-8k, 2023.
“NTK-Aware Scaled RoPE.” https://www.reddit.com/r/LocalLLaMA/comments/14lz7j5/ntkaware_scaled_rope_allows_llama_models_to_have/, 2023.
D. Kim, J. M. Paggi, C. Park, C. Bennett, and S. L. Salzberg, “Graph-based genome alignment and genotyping with hisat2 and hisat-genotype,” Nature biotechnology, vol. 37, pp. 907-905, 2019.
W. Meuleman, A. Muratov, E. Rynes, J. Halow, K. Lee, D. Bates, M. Diegel, D. Dunn, F. Neri, A. Teodosiadis, A. Reynolds, E. Haugen, J. Nelson, A. Johnson, M. Frerker, M. Buckley, R. Sandstrom, J. Vierstra, R. Kaul, and J. Stamatoyannopoulos, “Index and biological spectrum of human dnase hypersensitive sites,” Nature, vol. 584, pp. 244-251, 2020.

1. A method for determining locations of one or more genomic elements within a nucleotide sequence, the method comprising:
(a) receiving, by a processor of a computing device, nucleotide sequence data representing a sequence of a plurality of nucleotides;
(b) determining, by the processor, using a machine learning model and based on the nucleotide sequence data, a plurality of likelihood values,
wherein each likelihood value is associated with (i) a particular nucleotide of the sequence and (ii) a particular one of the one or more genomic element(s), and
wherein each likelihood value represents and/or quantifies a likelihood that the particular nucleotide is part of the particular genomic element with which the likelihood value is associated;
(c) determining and/or assigning, by the processor, one or more genomic element labels to each of at least a portion of the plurality of nucleotides, based at least in part on the plurality of likelihood values, thereby creating an annotated sequence data comprising the nucleotide sequence data together with the assigned genomic element labels; and
(d) storing, by the processor, the annotated sequence data and/or providing, by the processor, the annotated sequence data for display, and/or further processing.

2. The method of claim 1, wherein the nucleotide sequence data represents a deoxyribonucleic acid (DNA) sequence and/or a ribonucleic acid (RNA) sequence.

3. The method of claim 1, wherein the machine learning model receives as input and/or generates a tokenized representation of the sequence of the plurality of nucleotides.

4. The method of claim 1, wherein the nucleotide sequence data has a length of at least 100 kilobases (kb).

5. The method of claim 1, comprising:
sub-dividing the nucleotide sequence data into two or more partitions, each of the two or more partitions corresponding to a sub-sequence of the plurality of nucleotides; and
at step (b), using the machine learning model to determine a corresponding subset of the likelihood values for each partition.

6. The method of claim 1, wherein the one or more genomic elements comprise five (5) or more genomic elements.

7. The method of claim 1, wherein the one or more genomic elements comprise one or more gene elements.

8. The method of claim 1, wherein the one or more genomic elements comprise one or more regulatory elements.

9. The method of claim 1, wherein the one or more of the genomic elements are associated with a disease.

10. The method of claim 1, wherein the machine learning model comprises (i) an encoder and (ii) a segmentation head.

11. The method of claim 10, wherein the encoder is a pre-trained model, having been previously trained separately from the segmentation head.

12-13. (canceled)

14. The method of claim 10, wherein the encoder comprises (i) one or more convolutional layers and/or (ii) one or more transformer layers.

15. The method of claim 10, wherein step (b) comprises:
generating, via the encoder, one or more embeddings based on the nucleotide sequence data and/or a tokenized version thereof; and
determining, via the segmentation head, the plurality of likelihood values, based on the one or more embeddings.

16. (canceled)

17. The method of claim 10, wherein the encoder is or comprises a pre-trained neural network having been trained, at least in part in an un-supervised fashion using a training dataset comprising a plurality of example nucleotide sequences.

18. (canceled)

19. The method of claim 10, wherein the segmentation head is or comprises a convolutional neural network (CNN).

20-30. (canceled)

31. The method of claim 1, wherein step (c) comprises identifying, by the processor, one or more subsequence(s) within the nucleotide sequence data and determining, by the processor, an assigned genomic element label for each of the one or more subsequences based at least in part on the plurality of likelihood values.

32. The method of claim 1, wherein step (d) comprises using the annotated sequence data to develop a therapy.

33. The method of claim 1, wherein step (d) comprises using the annotated sequence data for detection, and/or prognosis of a diseases.

34. A method for determining locations of genomic elements within a nucleotide sequence, the method comprising:
(a) receiving, by a processor of a computing device, nucleotide sequence data representing a nucleotide sequence comprising a plurality of nucleotides;
(b) determining, by the processor, using a machine learning model and based on the nucleotide sequence data, a plurality of likelihood values,
wherein each likelihood value is associated with (i) a particular group of one or more nucleotides of the nucleotide sequence and (ii) a particular one of a plurality of genomic elements, and
wherein each likelihood value represents and/or quantifies a likelihood that at least a portion of the one or more nucleotides of the particular group is/are part of the particular one of the plurality of genomic elements with which it is associated;
(c) determining and/or assigning, by the processor, one or more genomic element labels to each of at least a portion of the plurality of nucleotides, based at least in part on the plurality of likelihood values, thereby creating an annotated sequence data comprising the nucleotide sequence data together with the assigned genomic element labels; and
(d) storing, by the processor, the annotated sequence data and/or providing, by the processor, the annotated sequence data for display, and/or further processing.

35. A method for determining locations of genomic elements within a genomic sequence, the method comprising:
(a) receiving, by a processor of a computing device, nucleotide sequence data representing a sequence comprising a plurality of nucleotides;
(b) determining, by the processor, using a machine learning model and based on the nucleotide sequence data, a plurality of likelihood values that measure a probability of each nucleotide of the sequence belonging to one or more of particular genomic elements, wherein the machine learning model comprises (i) an encoder model and (ii) a segmentation head;
(c) creating, by the processor, annotated sequence data comprising identifications of one or more genomic elements based on the likelihood values; and
(d) storing, by the processor, the annotated sequence data and/or providing, by the processor, the annotated sequence data for display, and/or further processing.

36-48. (canceled)

49. A system for determining locations of one or more genomic elements within a nucleotide sequence, the system comprising:
a processor of a computing device; and
memory having instructions stored thereon, wherein the instructions, when executed by the processor, cause the processor to:
(a) receive nucleotide sequence data representing a sequence of a plurality of nucleotides;
(b) determine, using a machine learning model and based on the nucleotide sequence data, a plurality of likelihood values,
wherein each likelihood value is associated with (i) a particular nucleotide of the sequence and (ii) a particular one of the one or more genomic element(s), and
wherein each likelihood value represents and/or quantifies a likelihood that the particular nucleotide is part of the particular genomic element with which the likelihood value is associated;
(c) determine and/or assign one or more genomic element labels to each of at least a portion of the plurality of nucleotides, based at least in part on the plurality of likelihood values, thereby creating an annotated sequence data comprising the nucleotide sequence data together with the assigned genomic element labels; and
(d) store the annotated sequence data and/or provide the annotated sequence data for display and/or further processing.

50. A system for determining locations of genomic elements within a nucleotide sequence, the system comprising:
a processor of a computing device; and
memory having instructions stored thereon, wherein the instructions, when executed by the processor, cause the processor to:
(a) receive nucleotide sequence data representing a nucleotide sequence comprising a plurality of nucleotides;
(b) determine, using a machine learning model and based on the nucleotide sequence data, a plurality of likelihood values,
wherein each likelihood value is associated with (i) a particular group of one or more nucleotides of the nucleotide sequence and (ii) a particular one of a plurality of genomic elements, and
wherein each likelihood value represents and/or quantifies a likelihood that at least a portion of the one or more nucleotides of the particular group is/are part of the particular one of the plurality of genomic elements with which it is associated;
(c) determine and/or assign one or more genomic element labels to each of at least a portion of the plurality of nucleotides, based at least in part on the plurality of likelihood values, thereby creating an annotated sequence data comprising the nucleotide sequence data together with the assigned genomic element labels; and
(d) store the annotated sequence data and/or provide the annotated sequence data for display and/or further processing.

51. A system for determining locations of genomic elements within a genomic sequence, the system comprising:
a processor of a computing device; and
memory having instructions stored thereon, wherein the instructions, when executed by the processor, cause the processor to:
(a) receive nucleotide sequence data representing a sequence comprising a plurality of nucleotides;
(b) determine, using a machine learning model and based on the nucleotide sequence data, a plurality of likelihood values that measure a probability of each nucleotide of the sequence belonging to one or more of particular genomic elements, wherein the machine learning model comprises (i) an encoder and (ii) a segmentation head;
(c) create annotated sequence data comprising identifications of one or more genomic elements based on the likelihood values; and
(d) store the annotated sequence data and/or provide the annotated sequence data for display and/or further processing.

52-80. (canceled)
